(19) United States
(12) Patent Application Publication
(10) Pub. No.: US 2022/0363649 A1
(43) Pub. Date: Nov. 17, 2022

(54) ORGANIC COMPOUNDS

(71) Applicant: Rutgers, The State University of New Jersey, New Brunswick, NJ (US)

(72) Inventors: Youyi Peng, New Brunswick, NJ (US); William J. Welsh, Piscataway, NJ (US)

(21) Appl. No.: 17/343,965

(22) Filed: Jun. 10, 2021

Related U.S. Application Data
(63) Continuation-in-part of application No. PCT/US2019/066048, filed on Dec. 12, 2019.
(60) Provisional application No. 62/778,498, filed on Dec. 12, 2018.

(51) Int. Cl. C07D 249/08; A61K 45/06; A61P 25/04; A61K 31/454; A61K 31/5377; A61K 31/496; C07D 401/06; A61K 31/4196; C07D 409/14; C07D 403/06; C07D 487/08; A61K 31/485

(52) U.S. Cl. CPC C07D 249/08; A61K 45/06; A61P 25/04; A61K 31/454; A61K 31/5377; A61K 31/485; C07D 401/06; A61K 31/4196; C07D 409/14; C07D 403/06; C07D 487/08; A61K 31/496

(57) ABSTRACT

The invention relates in one aspect to compounds, and compositions comprising such compounds, that can be used to treat and/or prevent pain in a subject. In certain embodiments, the subject is a mammal. In yet other embodiments, the mammal is human.

### CROSS-REFERENCE TO RELATED APPLICATION

The present application is a continuation-in-part of, and claims priority to International Application No. PCT/US2019/066048, filed Dec. 12, 2019, which claims priority under 35 U.S.C. § 119(e) to U.S. Provisional Patent Application No. 62/778,498, filed Dec. 12, 2018, all of which applications are incorporated herein by reference in their entireties.

### BACKGROUND OF THE INVENTION

The sigma receptors were initially identified in 1976 as an opioid receptor subtype and later as a phencyclidine binding receptor. Now it is widely recognized that sigma receptors comprise a unique family distinct from opioid and phencyclidine receptors, and are classified into two subtypes: sigma 1 receptor (SlR) and sigma 2 receptor (S2R).

The SiR has been cloned encoding a protein of 223 amino acids in diverse species including human, and has a molecular weight of 25.3 kDa. It is highly expressed in both central and peripheral nervous systems, and has been identified as a ligand-gated molecular chaperone protein within the endoplasmic reticulum (ER) and plasma membranes. On the other hand, the S2R was initially identified as progesterone receptor membrane component 1 (PGRMC1), but later in 2017 was reported as the translation of gene TMEM97. Although the molecular identify of the S2R is still ambiguous, there is overwhelming evidence demonstrating the S2R plays important roles in different types of cancer, neurological diseases, inflammatory and autoimmune diseases.

Recent research on the SiR has implicated its role in various pathological disorders. SiR knock-out (KO) mice have been shown to attenuate chemical-induced (e.g., formalin, capsaicin) and neuropathic (e.g., paclitaxel-induced) pain (Cendan, et al., 2005, Eur. J. Pharmacol. 511:73-4; Entrena, et al., 2009, Pain 143:252-61; Nieto, et al., 2012, J. Pain 13:1107-21; Nieto, et al., 2014, Mol. Pain 10:11), and to potentiate opioid (e.g., morphine, oxycodone) analgesia but not its side effects (e.g., dependence, tolerance, constipation) (Sanchez-Fernandez, et al., 2014, J.

Pharmacol. Exp. Ther. 348:32-45; Sanchez-Fernandez, et al., 2013, Neuropharmacology 70:348-58; Vidal-Torres, et al., 2013, Eur. J. Pharmacol. 711:63-72). Meanwhile, antagonists selective for the SiR have been shown to elicit beneficial effects in neuropathic pain, the enhancement of opioid analgesia, and the mitigation of drug abuse including alcohol, cocaine, and methamphetamine (Almansa, et al., 2014, Future Med. Chem. 6:1179-99; Arena, et al., 2018, Future Med. Chem. 10:231-256; Navarro, et al., 2010, Proc. Natl. Acad. Sci. U.S.A 107:18676-81). Emerging evidence also suggests that SiR antagonists inhibit tumor growth in prostate cancer xenograft models (Thomas, et al, 2017, Cancer Res. 77:2439-2452), and induce autophagic degradation of programmed death-ligand 1 (PD-L1) (Maher, et al., 2018, Mol. Cancer Res. 16:243-255). On the other hand, SiR agonists have been reported to play important roles in neuropsychiatric and neurodegenerative disorders including depression, amyotrophic lateral sclerosis, epilepsy, and Alzheimer's disease (Kulkarni, et al., 2009, Expert Rev.

Neurother. 9:1021-34; Jin, et al., 2015, Int. J. Clin. Exp. Med. 8:4808-20; Nguyen, et al., 2015, J. Pharmacol. Sci. 127:17-29).

Therefore there is an urgent need to develop novel compounds that bind to the sigma receptors (SiR and/or S2R). Such compounds can be used to treat certain diseases that are associated with SiR and/or S2R. The present invention addresses this unmet need.

### BRIEF SUMMARY OF THE INVENTION

The invention provides a compound of formula (I), or a salt, solvate, isotopically labeled, enantiomer, diastereomer, and/or tautomer thereof:

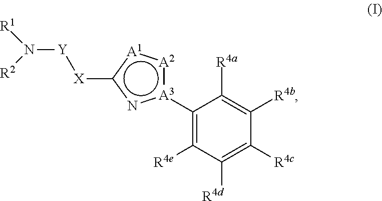

wherein R¹, R², A¹, A², A³, R^{4a}—R^{4e}, X, and Y are as defined elsewhere herein. The invention further provides a pharmaceutical composition comprising at least one pharmaceutically acceptable carrier and at least one compound contemplated herein. The invention further provides a method of increasing or decreasing activity of at least one sigma receptor selected from the group consisting of sigma 1 receptor (SiR) and sigma 2 receptor (S2R). The invention further provides a method of inhibiting, treating, and/or preventing pain in a subject. The invention further provides a method of inhibiting, treating, and/or preventing a disease or disorder contemplated herein in a subject.

In certain embodiments, the method comprises contacting the at least one sigma receptor with at least one compound and/or at least one pharmaceutical composition contemplated herein. In certain embodiments, the method comprises administering to the subject a therapeutically effective amount of at least one compound and/or at least one pharmaceutical composition contemplated herein. In certain embodiments, the disease or disorder is at least one selected from the group consisting of cancer and any other related proliferative diseases, neurological and neurodegenerative diseases, substance abuse disorders, depressive disorders, ocular disorders, and glaucoma.

### BRIEF DESCRIPTION OF THE DRAWINGS

The following detailed description of illustrative embodiments of the invention will be better understood when read in conjunction with the appended drawings. For the purpose of illustrating the invention, exemplary embodiments are shown in the drawings. It should be understood, however, that the invention is not limited to the precise arrangements and instrumentalities of the embodiments shown in the drawings.

FIG. 1 illustrates the finding that administering therapeutic levels of Compound 8 causes no motor incoordination in a rat rotarod test. 6 male Sprague-Dawley rats were used in each group. SEM bars are shown. Compound 8 was administered at two doses: 20 mg/kg and 40 mg/kg. No motor incoordination effect was observed at 20 mg/kg, and very slight and temporary motor incoordination effect at 40 mg/kg at t=60 min.

FIG. 2 illustrates the finding that Compound 8 exhibits dose-dependent analgesic activity in a rat formalin test at 20 and 40 mg/kg doses. 6 male Sprague-Dawley rats were used in each group. SEM bars are shown. Compound 8 was administered at two doses: 20 mg/kg and 40 mg/kg.

FIG. 3 illustrates the finding that Compound 8 exhibits analgesic activity in a Paclitaxel-induced cold hyperalgesia model in rats.

FIG. 4 illustrates the finding that Compound 8 exhibits analgesic activity in a Paclitaxel-induced mechanical allodynia model in rats.

FIG. 5 illustrates the finding that Compound 8 exhibits analgesic activity in a streptozotocin (STZ)-induced mechanical allodynia model in diabetic rats.

FIG. 6 illustrates the finding that Compound 8 exhibits analgesic activity in a STZ-induced thermal hyperalgesia model in diabetic rats.

FIG. 7 illustrates the finding that Compound 8 potentiates morphine's analgesia in a mouse model of acute pain (Tail Flick Test, n=8, i.p.).

FIG. 8 illustrates that compound 68 exhibits analgesic activity by attenuating the mechanical allodynia in spinal nerve ligation (SNL) Chung model in rats.

FIG. 9 illustrates that compound 68 exhibits analgesic activity by attenuating the thermal hyperalgesia in spinal nerve ligation (SNL) Chung model in rats.

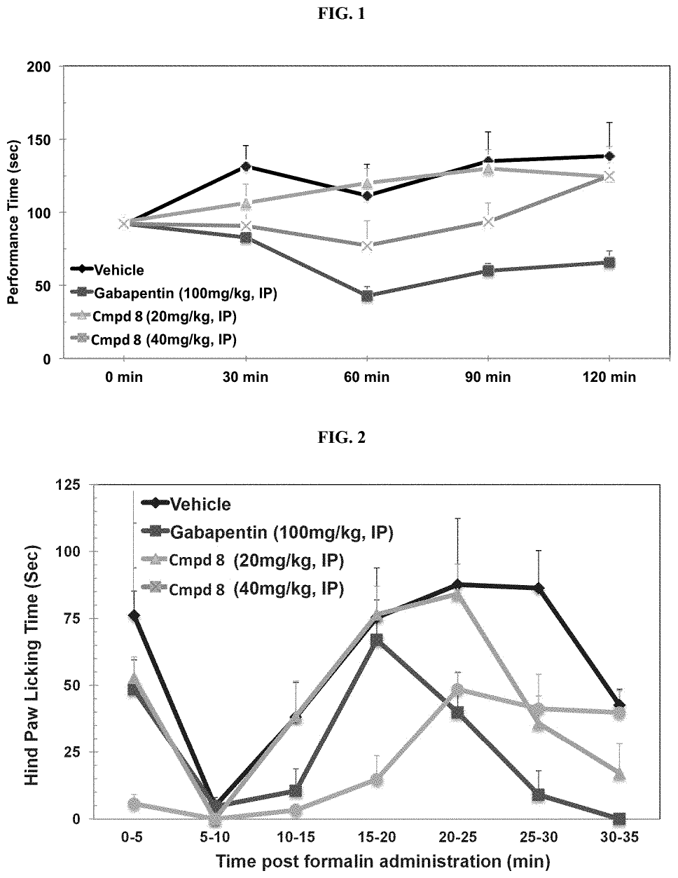

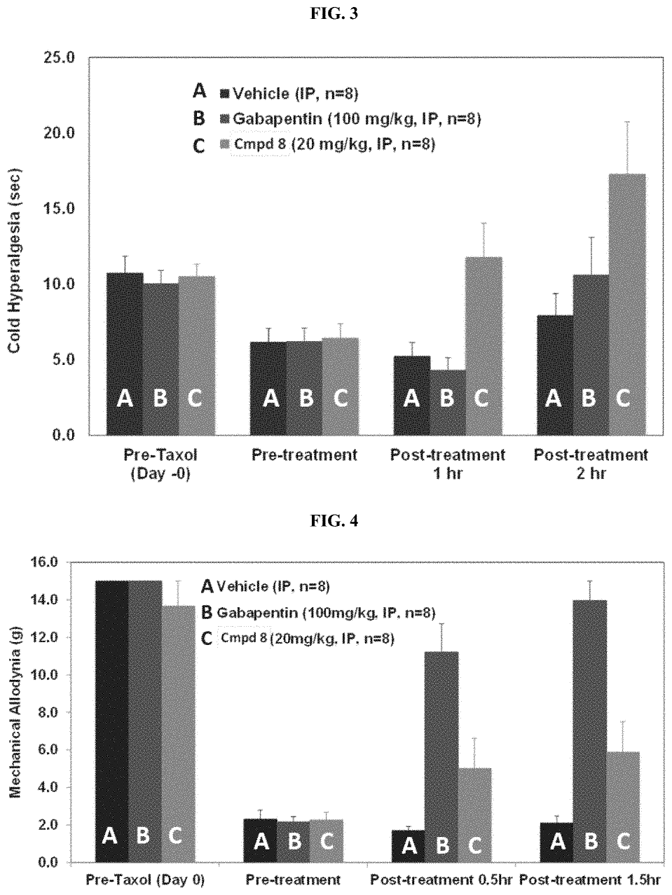

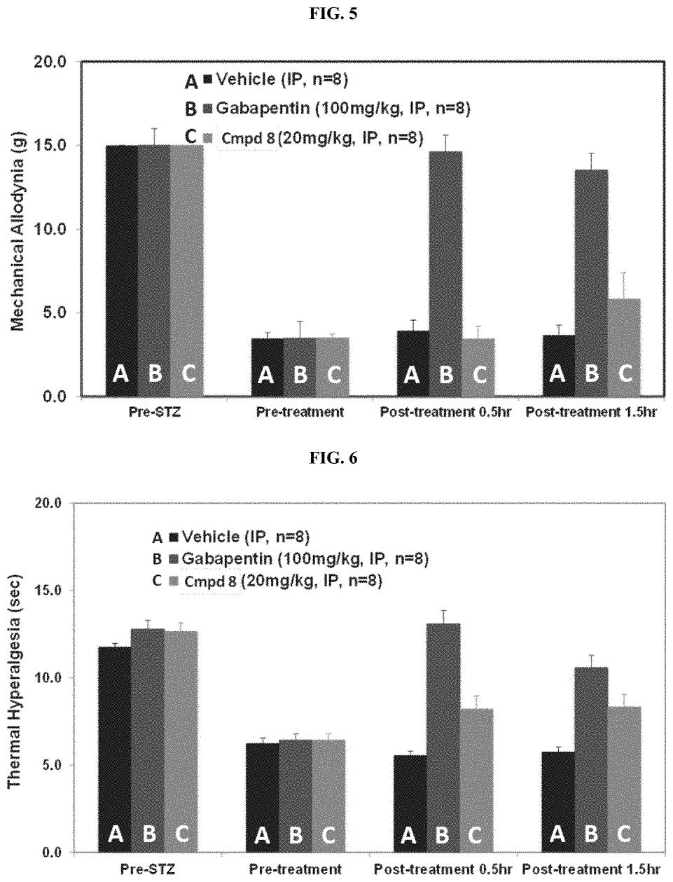

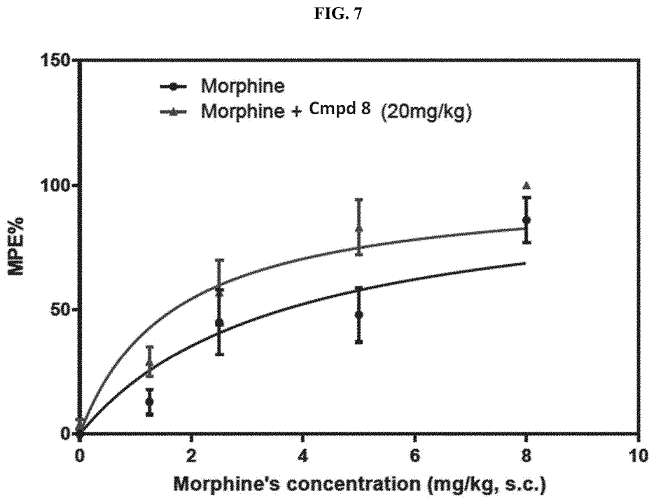

### DETAILED DESCRIPTION OF THE INVENTION

The invention relates in one aspect to compounds, and compositions comprising such compounds, that can be used to treat and/or prevent a sigma receptor-related diseases or disorder in a subject. In certain embodiments, the subject is a mammal. In yet other embodiments, the mammal is human.

### Definitions

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing act X and a claimed act of doing act Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

In one aspect, the terms “co-administered” and “co-administration” as relating to a subject refer to administering to the subject a compound of the invention or salt thereof along with a compound that may also treat any disease or disorder contemplated herein and/or with a compound that is useful in treating other medical conditions but which in themselves may cause or facilitate any disease or disorder contemplated herein. In certain embodiments, the co-administered compounds are administered separately, or in any kind of combination as part of a single therapeutic approach. The co-administered compound may be formulated in any kind of combinations as mixtures of solids and liquids under a variety of solid, gel, and liquid formulations, and as a solution.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, the term “Ki” or “Kᵢ” refers to the binding constant, which is a particular case of the equilibrium constant, that is associated with the binding and unbinding reaction of the sigma receptor (SR) and either the S1R and S2R ligand (L) molecules, which is formalized as: R+L [symbol] RL.

As used herein, the term “pharmaceutical composition” or “composition” refers to a mixture of at least one compound useful within the invention with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

The term “prevent,” “preventing,” or “prevention,” as used herein, means avoiding or delaying the onset of symptoms associated with a disease or condition in a subject that has not developed such symptoms at the time the administering of an agent or compound commences. Disease, condition, and disorder are used interchangeably herein.

By the term “specifically bind” or “specifically binds,” as used herein, is meant that a first molecule preferentially binds to a second molecule (e.g., a particular receptor or enzyme), but does not necessarily bind only to that second molecule.

As used herein, a “subject” may be a human or non-human mammal or a bird. Non-human mammals include, for example, livestock and pets, such as ovine, bovine, porcine, canine, feline and murine mammals. In certain embodiments, the subject is human.

The term “treat,” “treating,” or “treatment,” as used herein, means reducing the frequency or severity with which symptoms of a disease or condition are experienced by a subject by virtue of administering an agent or compound to the subject.

As used herein, the term “alkyl,” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆)alkyl, such as, but not limited to, ethyl, methyl, isopropyl, isobutyl, n-pentyl, n-hexyl and cyclopropylmethyl.

As used herein, the term “alkylene” by itself or as part of another substituent means, unless otherwise stated, a straight or branched hydrocarbon group having the number of carbon atoms designated (i.e., C₁-C₁₀ means one to ten carbon atoms) and includes straight, branched chain, or cyclic substituent groups, wherein the group has two open valencies. Examples include methylene, 1,2-ethylene, 1,1-ethylene, 1,1-propylene, 1,2-propylene and 1,3-propylene.

As used herein, the term “cycloalkyl,” by itself or as part of another substituent means, unless otherwise stated, a cyclic chain hydrocarbon having the number of carbon atoms designated (i.e., C₃-C₆ means a cyclic group comprising a ring group consisting of three to six carbon atoms) and includes straight, branched chain or cyclic substituent groups. Examples include cyclopropyl, cyclobutyl, cyclopentyl, cyclohexyl, cycloheptyl, and cyclooctyl. Most preferred is (C₃-C₆)cycloalkyl, such as, but not limited to, cyclopropyl, cyclobutyl, cyclopentyl and cyclohexyl.

As used herein, the term “alkenyl,” employed alone or in combination with other terms, means, unless otherwise stated, a stable mono-unsaturated or di-unsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkynyl,” employed alone or in combination with other terms, means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C≡CH. The term “homopropargylic” refers to a group exemplified by —CH₂CH₂—C≡CH. The term “substituted propargylic” refers to a group exemplified by —CR₂—C≡CR, wherein each occurrence of R is independently H, alkyl, substituted alkyl, alkenyl or substituted alkenyl, with the proviso that at least one R group is not hydrogen.

The term “substituted homopropargylic” refers to a group exemplified by —CR₂CR₂—C≡CR, wherein each occurrence of R is independently H, alkyl, substituted alkyl, alkenyl or substituted alkenyl, with the proviso that at least one R group is not hydrogen.

As used herein, the term “substituted alkyl,” “substituted cycloalkyl,” “substituted alkenyl” or “substituted alkynyl” means alkyl, cycloalkyl, alkenyl or alkynyl, as defined above, substituted by one, two or three substituents selected from the group consisting of halogen, C₁-C₆ alkoxy, C₁-C₆ haloalkyl, and —OH.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃)alkoxy, such as, but not limited to, ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “heteroalkenyl” by itself or in combination with another term means, unless otherwise stated, a stable straight or branched chain monounsaturated or di-unsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, and —CH₂—CH═CH—CH₂—SH.

As used herein, the term “aromatic” refers to a carbocycle or heterocycle with one or more polyunsaturated rings and having aromatic character, i.e. having (4n+2) delocalized 1r (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “aryl-(C₁-C₃)alkyl” means a functional group wherein a one to three carbon alkylene chain is attached to an aryl group, e.g., —CH₂CH₂-phenyl or —CH₂-phenyl (benzyl). Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl. Non-limiting examples of heterocyclyl include pyrrolidinyl, piperidinyl, morpholinyl, thiomorpholinyl, piperazinyl, alkyl piperidinyl, cyclohexamino, and azepanyl.

As used herein, the term “heteroaryl” or “heteroaromatic” refers to a heterocycle having aromatic character. A polycyclic heteroaryl may include one or more rings that are partially saturated. Examples include benzothiophene, benzofuran, and indole

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

In certain embodiments, each occurrence of the heteroaryl is independently selected from the group consisting of quinolinyl, imidazo[1,2-a]pyridyl, pyridyl, pyrimidyl, pyrazinyl, imidazolyl, thiazolyl, pyrazolyl, isoxazolyl, indolyl (such as, but not limited to, 2-, 3-, 4-, 5-, 6- and 7-indolyl), oxadiazolyl (including 1,2,3-, 1,2,4-, 1,2,5-, and 1,3,4-oxadiazole), and triazolyl (such as 1,2,3-triazolyl and 1,2,4-triazolyl).

The aforementioned listing of heterocyclyl and heteroaryl moieties is intended to be representative and not limiting.

As used herein, the term “substituted” means that an atom or group of atoms has replaced hydrogen as the substituent attached to another group.

For aryl, aryl-(C₁-C₃)alkyl and heterocyclic groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet other embodiments, the substituents vary in number between one and two. In yet other embodiments, the substituents are independently selected from the group consisting of C₁-C₆ alkyl, —OH, C₁-C₆ alkoxy, halo, amino, acetamido, and nitro. As used herein, where a substituent is an alkyl or alkoxy group, the carbon chain may be branched, straight or cyclic, with straight being preferred.

Throughout this disclosure, various aspects of the invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The invention provides compounds that bind to at least one sigma receptor selected from the group consisting of SIR and S2R. In certain embodiments, the compounds of the invention bind to SiR selectively over S2R. In certain embodiments, the compounds of the invention bind to SiR and S2R with equal, or nearly equal, affinity. In certain embodiments, the compounds of the invention bind to S2R selectively over SiR.

The compounds of the invention can be used to treat, prevent, and/or ameliorate pain in a subject. In certain embodiments, the pain comprises chronic pain. In certain embodiments, the pain comprises acute pain. In other embodiments, the pain comprises neuropathic pain. In yet other embodiments, the pain comprises nociceptive pain. In yet other embodiments, the pain comprises hyperalgesia. In yet other embodiments, the pain comprises allodynia.

In certain embodiments, the compounds of the invention diminish pain without producing respiratory depression, which is a side effect characteristic of morphine and other well-known and widely used opioids and narcotics. In certain embodiments, the compounds of the invention diminish pain without producing constipation, which is a side effect characteristic of morphine and other well-known and widely used opioids and narcotics. In certain embodiments, the compounds of the invention diminish pain without producing nausea, which is a side effect characteristic of morphine and other well-known and widely used opioids and narcotics. In certain embodiments, the compounds of the invention diminish pain without producing significant (or any at all) tolerance, which is a side effect characteristic of morphine and other well-known and widely used opioids and narcotics. In certain embodiments, the compounds of the invention diminish pain without producing significant (or any at all) tachyphylaxis, which is a side effect characteristic of morphine and other well-known and widely used opioids and narcotics. In certain embodiments, the compounds of the invention diminish pain without producing emesis, which is a side effect characteristic of morphine and other well-known and widely used opioids and narcotics. In certain embodiments, the compounds of the invention diminish pain without producing withdrawal adverse effects, which is a side effect characteristic of morphine and other well-known and widely used opioids and narcotics. In certain embodiments, the compounds of the invention diminish pain without producing dependence, which is a side effect characteristic of morphine and other well-known and widely used opioids and narcotics. In certain embodiments, the compounds of the invention diminish pain without producing one or more of the following phenomena: nausea, emesis, constipation, respiratory depression, tolerance, tachyphylaxis, dependence and/or addiction.

In certain embodiments, the compounds and compositions of the invention promote neuroprotection in a subject. In certain embodiments, the compounds and compositions of the invention inhibit, treat, and/or prevent neurological and neurodegenerative diseases, such as but not limited to Alzheimer's disease-type dementia, motor neuron disease, and/or amyotrophic lateral sclerosis (ALS). In certain embodiments, the compounds and compositions of the invention improve learning and/or memory in a subject.

In certain embodiments, the compounds and compositions of the invention inhibit, treat, and/or prevent cancer and related proliferative diseases.

In certain embodiments, the compounds and compositions of the invention inhibit, treat, and/or prevent substance abuse disorders in a subject. In certain embodiments, the compounds and compositions of the invention inhibit, treat, and/or prevent depressive disorders in a subject. In certain embodiments, the compounds and compositions of the invention inhibit, treat, and/or prevent ocular disorder and/or glaucoma in a subject.

In certain non-limiting embodiments, the binding selectivity of the compound of the invention for S2R over S1R is equal to, or about, 1.

In certain non-limiting embodiments, the binding selectivity of the compound of the invention for S1R over S2R is greater than 1. In other non-limiting embodiments, the binding selectivity of the compound of the invention for S1R over S2R is equal to or greater than about 1.1, about 1.2, about 1.5, about 2, about 3, about 4, about 5, about 6, about 7, about 8, about 9, about 10, about 12, about 14, about 16, about 18, about 20, about 22, about 24, about 26, about 28, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 250, about 300, about 350, about 400, about 450, about 500, about 550, about 600, about 650, about 700, about 750, about 800, about 850, about 900, about 950, about 1,000, about 2,000, about 3,000, about 4,000, about 5,000, about 6,000, about 7,000, about 8,000, about 9,000, about 10,000, about 20,000, about 30,000, about 40,000, and/or about 50,000.

In certain non-limiting embodiments, the binding selectivity of the compound of the invention for S2R over S1R is greater than 1. In other non-limiting embodiments, the binding selectivity of the compound of the invention for S2R over S1R is equal to or greater than about 1.1, about 1.2, about 1.5, about 2, about 3, about 4, about 5, about 6, about 7, about 8, about 9, about 10, about 12, about 14, about 16, about 18, about 20, about 22, about 24, about 26, about 28, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 250, about 300, about 350, about 400, about 450, about 500, about 550, about 600, about 650, about 700, about 750, about 800, about 850, about 900, about 950, about 1,000, about 2,000, about 3,000, about 4,000, about 5,000, about 6,000, about 7,000, about 8,000, about 9,000, about 10,000, about 20,000, about 30,000, about 40,000, and/or about 50,000.

### Compounds and Compositions

The invention includes a compound of formula (I), or a salt, solvate, isotopically labeled (such as, for example, deuterated at one or more positions), enantiomer, diastereomer, and/or tautomer thereof:

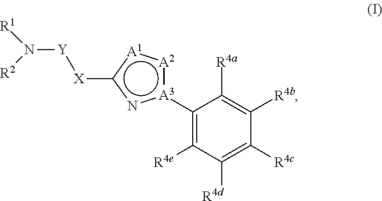

wherein:

A¹, A², and A³ are such that one applies: (a) A¹=N, A²=NR³, and A³=C; (b) A¹=NR³, A²=N, and A³=C; or (c) A¹=N, A²=CR³, and A³=N;

R¹ is selected from the group consisting of H, C₁-C₈ alkyl, C₂-C₈ alkenyl, C₂-C₈ alkynyl, or C₃-C₁₀ cycloalkyl, wherein each alkyl, alkenyl, alkynyl, or cycloalkyl is independently optionally substituted with at least one selected from the group consisting of F, Cl, Br, I, C₁-C₈ alkyl, C₁-C₈ haloalkyl, and C₃-C₈ cycloalkyl;

R² is selected from the group consisting of H, C₁-C₈ alkyl, C₂-C₈ alkenyl, C₂-C₈ alkynyl, or C₃-C₁₀ cycloalkyl, wherein each alkyl, alkenyl, alkynyl, or cycloalkyl is independently optionally substituted with at least one selected from the group consisting of F, Cl, Br, I, C₁-C₈ alkyl, C₁-C₈ haloalkyl, and C₃-C₈ cycloalkyl;
or R¹ and R² combine with the N atom to which they are bound to form 3- to 8-membered heterocyclyl,
   wherein each heterocyclyl is independently optionally substituted with at least one substituent selected from the group consisting of F, Cl, Br, I, C₁-C₈ alkyl, C₁-C₈ haloalkyl, C₂-C₈ alkenyl, C₂-C₈ alkenyl, phenyl, heteroaryl, and (aryl)-C₁-C₆ alkyl, wherein two substituents bound to the same atom of the heterocyclyl can combine to form C₂-C₇ alkylene (thus yielding a spiro bicyclic heterocyclyl) which is optionally substituted with at least one substituent selected from the group consisting of F, Cl, Br, I, C₁-C₈ alkyl, C₁-C₈ haloalkyl, C₂-C₈ alkenyl, C₂-C₈ alkenyl, phenyl, heteroaryl, and (aryl)-C₁-C₆ alkyl, and wherein two substituents bound to distinct atoms of the heterocyclyl can combine to form C₁-C₇ alkylene (thus yielding a fused or bridged bicyclic heterocyclyl) which is optionally substituted with at least one substituent selected from the group consisting of F, Cl, Br, I, C₁-C₈ alkyl, C₁-C₈ haloalkyl, C₂-C₈ alkenyl, C₂-C₈ alkenyl, phenyl, heteroaryl, and (aryl)-C₁-C₆ alkyl;

Y is —(CH₂)ₙ—, wherein n is 0, 1, 2, 3, 4, 5, or 6, and wherein each CH₂ group in Y is independently optionally substituted with at least one selected from the group consisting of C₁-C₆ alkyl, C₁-C₆ haloalkyl, F, Cl, Br, and I;
X is selected from the group consisting of —CH₂—, —O—, —NH—, —N(C₁-C₆ alkyl)-, and —S—, wherein if n is 0, then X is —CH₂—, and wherein each CH₂ group in X is independently optionally substituted with at least one selected from the group consisting of C₁-C₆ alkyl, C₁-C₆ haloalkyl, F, Cl, Br, and I;

R³ is selected from the group consisting of H, C₁-C₄ alkyl, C₂-C₄ alkenyl, C₂-C₄ alkynyl, and C₃-C₆ cycloalkyl, wherein each alkyl, alkenyl, alkynyl or cycloalkyl is independently optionally substituted with at least one selected from the group consisting of C₁-C₄ alkyl, C₁-C₄ haloalkyl, F, Cl, Br, and I;

each of R^{4a}—R⁴ is independently selected from the group consisting of H, F, Cl, Br, I, C₁-C₈ alkyl, C₁-C₈ haloalkyl, C₁-C₈ haloalkoxy, C₂-C₈ alkenyl, C₂-C₈ alkynyl, C₃-C₁₀ cycloalkyl, phenyl, phenoxy, and benzyloxy, wherein each alkyl, alkenyl, alkynyl, cycloalkyl, phenyl, or benzyl is independently optionally substituted with at least one selected from the group consisting of C₁-C₆ alkyl, C₁-C₆ haloalkyl, F, Cl, Br, and I,
or R^{4c} and R^{4d} combine with the atoms to which they are bound to form C₄-C₈ cycloalkyl, phenyl, or heterocyclyl, wherein each cycloalkyl, phenyl, heterocyclyl, or heteroaryl is independently substituted with at least one substituent selected from the group consisting of F, Cl, Br, I, C₁-C₈ alkyl, C₁-C₈ haloalkyl, C₂-C₈ alkenyl, and C₂-C₈ alkynyl; and wherein the heterocyclyl is selected from the group consisting of pyrrolyl, furanyl and thiophenyl.

In certain embodiments, —NR¹R² is an optionally substituted heterocyclyl selected from the group consisting of pyrrolidinyl, piperidinyl, morpholinyl, thiomorpholinyl, piperazinyl, N—(C₁-C₆ alkyl) piperidinyl, cyclohexyl-amino, and azepanyl.

In certain embodiments, the compound of formula (I) is a compound of formula (Ia):

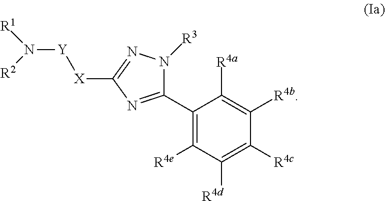

In certain embodiments, the compound of formula (I) is a compound of formula (Ib):

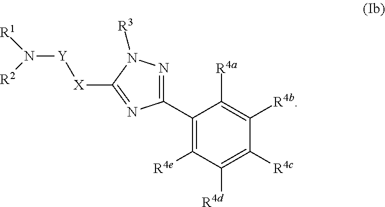

In certain embodiments, the compound of formula (I) is a compound of formula (Ic):

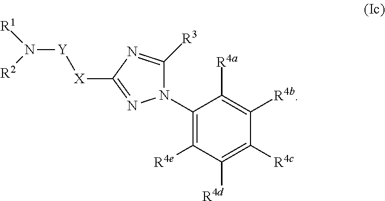

In certain embodiments, —NR¹R² is

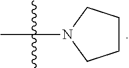 [SMILES: *N1CCCC1]

In certain embodiments, —NR¹R² is

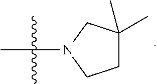 [SMILES: *N1CCC(C)(C)C1]

In certain embodiments, —NR¹R² is

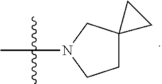 [SMILES: *N1CCC2(CC2)C1]

In certain embodiments, —NR¹R² is

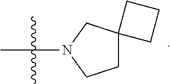 [SMILES: *N1CCC2(CCC2)C1]

In certain embodiments, —NR¹R² is

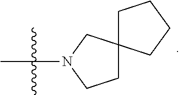 [SMILES: *N1CCC2(CCCC2)C1]

In certain embodiments, —NR¹R² is

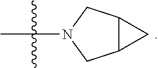 [SMILES: *N1CC2CC2C1]

In certain embodiments, —NR¹R² is

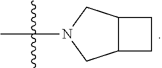 [SMILES: *N1CC2CCC2C1]

In certain embodiments, —NR¹R² is

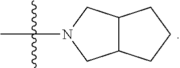 [SMILES: *N1CC2CCCC2C1]

In certain embodiments, —NR¹R² is

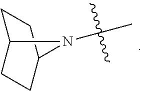 [SMILES: *N1C2CCC1CC2]

In certain embodiments, —NR¹R² is

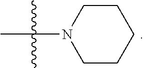 [SMILES: *N1CCCCC1]

In certain embodiments, —NR¹R² is

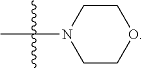 [SMILES: *N1CCOCC1]

In certain embodiments, —NR¹R² is

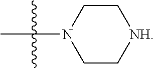 [SMILES: *N1CCNCC1]

In certain embodiments, —NR¹R² is

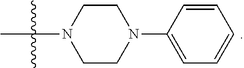 [SMILES: *N1CCN(c2ccccc2)CC1]

In certain embodiments, —NR¹R² is

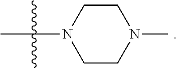 [SMILES: *N1CCN(C)CC1]

In certain embodiments, —NR¹R² is

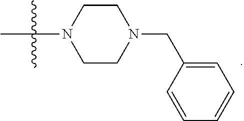 [SMILES: *N1CCN(Cc2ccccc2)CC1]

In certain embodiments, —NR¹R² is

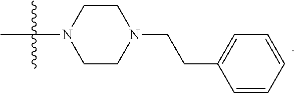 [SMILES: *N1CCN(CCc2ccccc2)CC1]

In certain embodiments, —NR¹R² is

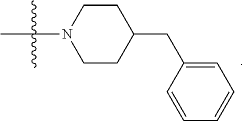 [SMILES: *N1CCC(Cc2ccccc2)CC1]

In certain embodiments, —NR¹R² is

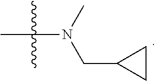 [SMILES: *N(C)CC1CC1]

In certain embodiments, —NR¹R² is

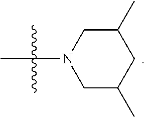 [SMILES: *N1CC(C)CC(C)C1]

In certain embodiments, —NR¹R² is

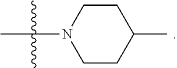 [SMILES: *N1CCC(C)CC1]

In certain embodiments, —NR¹R² is

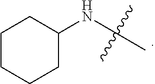 [SMILES: *NC1CCCCC1]

In certain embodiments, —NR¹R² is

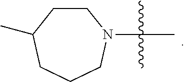 [SMILES: *N1CCCC(C)CC1]

In certain embodiments, —NR¹R² is

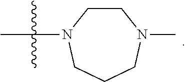 [SMILES: *N1CCCN(C)CC1]

In certain embodiments, NR¹R² is

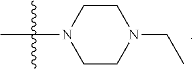 [SMILES: *N1CCN(CC)CC1]

In certain embodiments, NR¹R² is

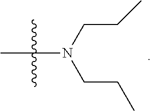 [SMILES: *N(CCC)CCC]

In certain embodiments, NR¹R² is

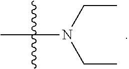 [SMILES: *N(CC)CC]

In certain embodiments, NR¹R² is

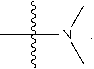 [SMILES: *N(C)C]

In certain embodiments, NR¹R² is

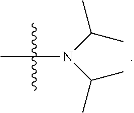 [SMILES: *N(C(C)C)C(C)C]

In certain embodiments, NR¹R² is

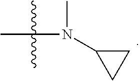 [SMILES: *N(C)C1CC1]

In certain embodiments, NR¹R² is

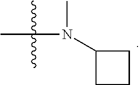 [SMILES: *N(C)C1CCC1]

In certain embodiments, NR¹R² is

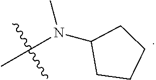 [SMILES: *N(C)C1CCCC1]

In certain embodiments, NR¹R² is

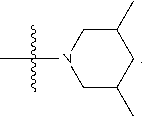 [SMILES: *N1CC(C)CC(C)C1]

In certain embodiments, —NR¹R² is

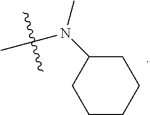 [SMILES: *N(C)C1CCCCC1]

In certain embodiments,

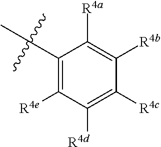

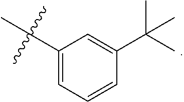 [SMILES: *c1cccc(C(C)(C)C)c1]

In certain embodiments,

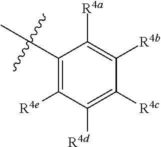

is

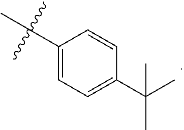 [SMILES: *c1ccc(C(C)(C)C)cc1]

In certain embodiments

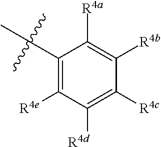

is

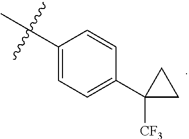 [SMILES: *c1ccc(C2(C(F)(F)F)CC2)cc1]

In certain embodiments,

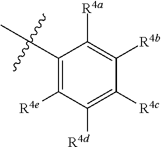

is

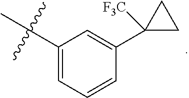 [SMILES: *c1cccc(C2(C(F)(F)F)CC2)c1]

In certain embodiments,

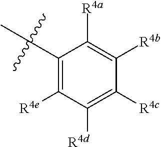

is

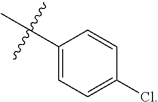 [SMILES: *c1ccc(Cl)cc1]

In certain embodiments,

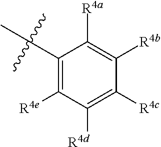

is

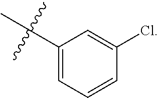 [SMILES: *c1cccc(Cl)c1]

In certain embodiments,

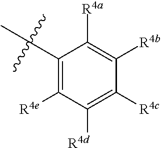

is

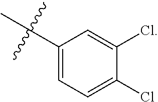 [SMILES: *c1ccc(Cl)c(Cl)c1]

In certain embodiments,

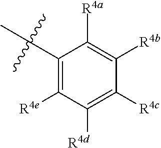

is

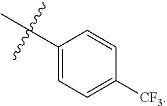 [SMILES: *c1ccc(C(F)(F)F)cc1]

In certain embodiments,

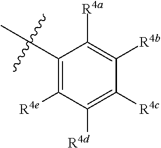

is

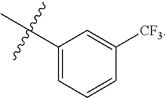 [SMILES: *c1cccc(C(F)(F)F)c1]

In certain embodiments,

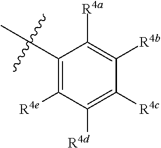

is

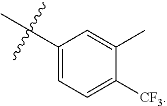 [SMILES: *c1ccc(C(F)(F)F)c(C)c1]

In certain embodiments,

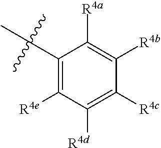

is

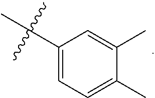 [SMILES: *c1ccc(C)c(C)c1]

In certain embodiments,

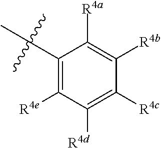

is

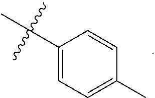 [SMILES: *c1ccc(C)cc1]

In certain embodiments,

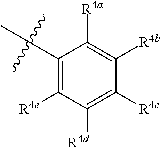

is

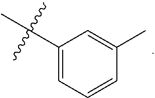 [SMILES: *c1cccc(C)c1]

In certain embodiments,

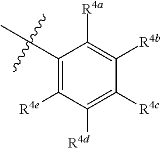

is

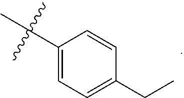 [SMILES: *c1ccc(CC)cc1]

In certain embodiments,

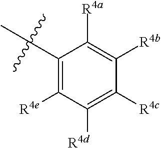

is

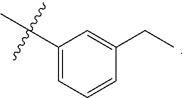 [SMILES: *c1cccc(CC)c1]

In certain embodiments,

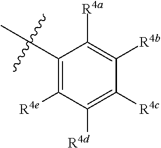

is

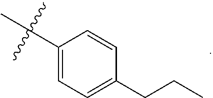 [SMILES: *c1ccc(CCC)cc1]

In certain embodiments,

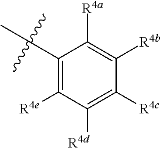

is.

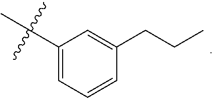 [SMILES: *c1cccc(CCC)c1]

In certain embodiments,

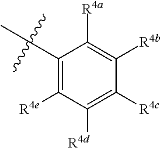

is

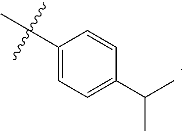 [SMILES: *c1ccc(C(C)C)cc1]

In certain embodiments,

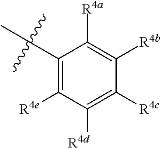

is

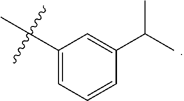 [SMILES: *c1cccc(C(C)C)c1]

In certain embodiments,

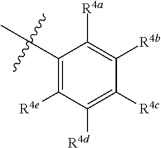

is

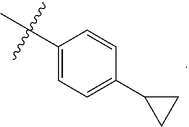 [SMILES: *c1ccc(C2CC2)cc1]

In certain embodiments,

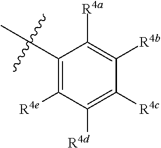

is

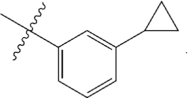 [SMILES: *c1cccc(C2CC2)c1]

In certain embodiments,

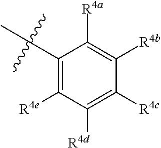

is

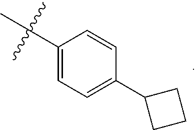 [SMILES: *c1ccc(C2CCC2)cc1]

In certain embodiments,

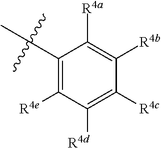

is

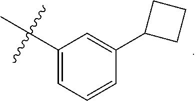 [SMILES: *c1cccc(C2CCC2)c1]

In certain embodiments,

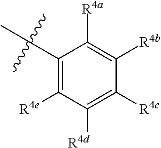

is

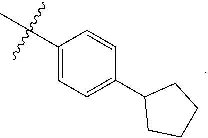 [SMILES: *c1ccc(C2CCCC2)cc1]

In certain embodiments

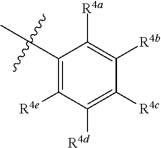

is

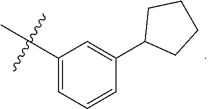 [SMILES: *c1cccc(C2CCCC2)c1]

In certain embodiments,

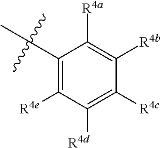

is

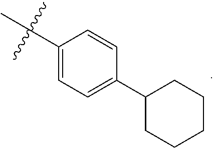 [SMILES: *c1ccc(C2CCCCC2)cc1]

In certain embodiments,

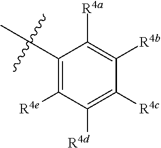

is

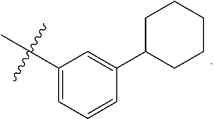 [SMILES: *c1cccc(C2CCCCC2)c1]

In certain embodiments,

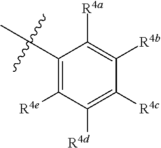

is

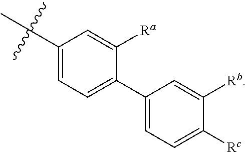

In certain embodiments,

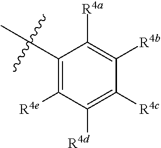

is

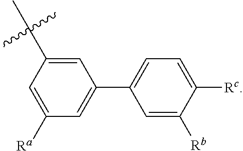

In certain embodiments

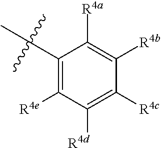

is

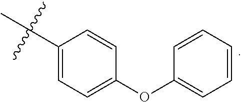 [SMILES: *c1ccc(Oc2ccccc2)cc1]

certain embodiments,

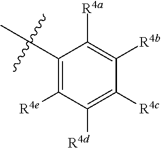

is

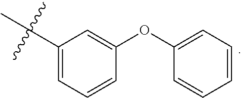 [SMILES: *c1cccc(Oc2ccccc2)c1]

In certain embodiments,

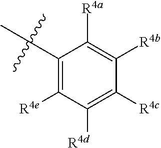

is

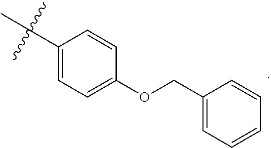 [SMILES: *c1ccc(OCc2ccccc2)cc1]

In certain embodiments,

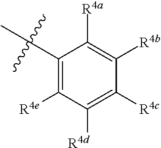

is

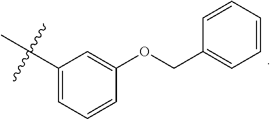 [SMILES: *c1cccc(OCc2ccccc2)c1]

In certain embodiments,

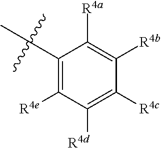

is

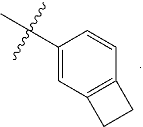 [SMILES: *c1ccc2c(c1)CC2]

In certain embodiments

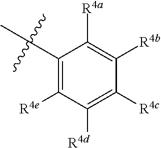

is

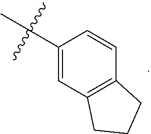 [SMILES: *c1ccc2c(c1)CCC2]

In certain embodiments,

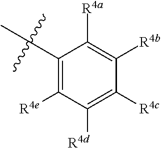

is

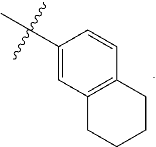 [SMILES: *c1ccc2c(c1)CCCC2]

In certain embodiments

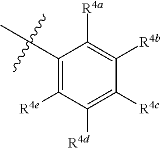

is

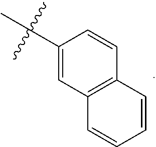 [SMILES: *c1ccc2ccccc2c1]

In certain embodiments,

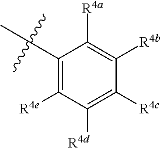

is

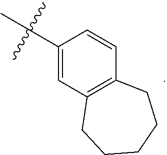 [SMILES: *c1ccc2c(c1)CCCCC2]

In certain embodiments,

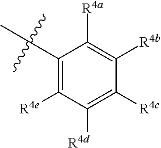

is

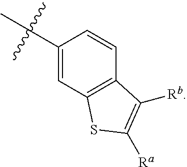

In certain embodiments,

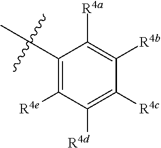

is

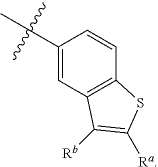

In certain embodiments,

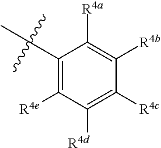

is

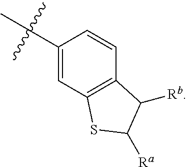

In certain embodiments,

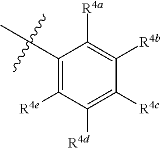

is

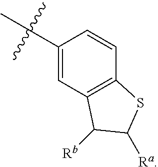

In certain embodiments

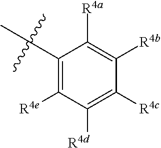

is

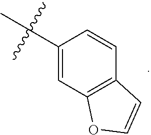 [SMILES: *c1ccc2ccoc2c1]

In certain embodiments,

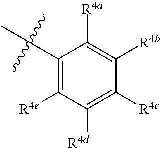

is

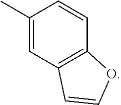

In certain embodiments,

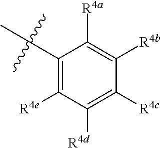

is

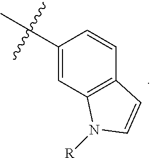

In certain embodiments,

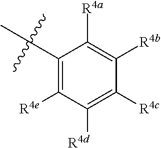

is

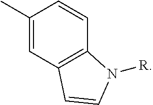

In certain embodiments, each occurrence of R is independently H, C₁-C₆ alkyl, C₁-C₆ haloalkyl, and C₃-C₈ cycloalkyl. In certain embodiments, each occurrence of R^{a}, R^{b}, and R^{c} is independently H or C₁.

In certain embodiments, R¹ and R² are each —CH₃.

In certain embodiments, R³ is methyl. In certain embodiments, R³ is ethyl. In certain embodiments, R³ is isopropyl. In certain embodiments, R³ is cyclopropyl.

In certain embodiments, the compound is 1-((5-(4-(tert-butyl)phenyl)-1-methyl-1H-1,2,4-triazol-3-yl)methyl)piperidine, or a salt, solvate, isotopically labeled, enantiomer, diastereomer, and/or tautomer thereof. In certain embodiments, the compound is 4-((5-(4-(tert-butyl)phenyl)-1-methyl-1H-1,2,4-triazol-3-yl)methyl)morpholine, or a salt, solvate, isotopically labeled, enantiomer, diastereomer, and/or tautomer thereof. In certain embodiments, the compound is 1-((5-(4-(tert-butyl)phenyl)-1-methyl-1H-1,2,4-triazol-3-yl)methyl)-4-methylpiperazine, or a salt, solvate, isotopically labeled, enantiomer, diastereomer, and/or tautomer thereof. In certain embodiments, the compound is 1-((5-(4-(tert-butyl)phenyl)-1-methyl-1H-1,2,4-triazol-3-yl)methyl)-4-phenylpiperazine, or a salt, solvate, isotopically labeled, enantiomer, diastereomer, and/or tautomer thereof. In certain embodiments, the compound is 1-((5-(4-(tert-butyl)phenyl)-1-methyl-1H-1,2,4-triazol-3-yl)methyl)-4-phenethylpiperazine, or a salt, solvate, isotopically labeled, enantiomer, diastereomer, and/or tautomer thereof. In certain embodiments, the compound is 1-benzyl-4-((5-(4-(tert-butyl)phenyl)-1-methyl-1H-1,2,4-triazol-3-yl)methyl)piperazine, or a salt, solvate, isotopically labeled, enantiomer, diastereomer, and/or tautomer thereof. In certain embodiments, the compound is 4-benzyl-1-((5-(4-(tert-butyl)phenyl)-1-methyl-1H-1,2,4-triazol-3-yl)methyl)piperidine, or a salt, solvate, isotopically labeled, enantiomer, diastereomer, and/or tautomer thereof. In certain embodiments, the compound is 5-(4-(tert-butyl)phenyl)-1-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 1-(5-(4-(tert-butyl)phenyl)-1-methyl-1H-1,2,4-triazol-3-yl)-N-(cyclopropylmethyl)-N-methylmethanamine, or a salt, solvate, isotopically labeled, enantiomer, diastereomer, and/or tautomer thereof. In certain embodiments, the compound is 1-methyl-3-(pyrrolidin-1-ylmethyl)-5-(4-(1-(trifluoromethyl)cyclopropyl)phenyl)-1H-1,2,4-triazole, or a salt, solvate, isotopically labeled, enantiomer, diastereomer, and/or tautomer thereof. In certain embodiments, the compound is 5-(3,4-dichlorophenyl)-1-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 1-methyl-3-(pyrrolidin-1-ylmethyl)-5-(4-(trifluoromethyl)phenyl)-1H-1,2,4-triazole, or a salt, solvate, isotopically labeled, enantiomer, diastereomer, and/or tautomer thereof. In certain embodiments, the compound is 1-methyl-5-(3-methyl-4-(trifluoromethyl)phenyl)-3-(pyrrolidin-1-ylmethyl)-1H-1,2,4-triazole, or a salt, solvate, isotopically labeled, enantiomer, diastereomer, and/or tautomer thereof. In certain embodiments, the compound is 5-(4-isopropylphenyl)-1-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 1-methyl-3-(pyrrolidin-1-ylmethyl)-5-(5,6,7,8-tetrahydronaphthalen-2-yl)-1H-1,2,4-triazole, or a salt, solvate, isotopically labeled, enantiomer, diastereomer, and/or tautomer thereof. In certain embodiments, the compound is 5-(3-(tert-butyl)phenyl)-1-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 1-methyl-3-(pyrrolidin-1-ylmethyl)-5-(3-(1-(trifluoromethyl)cyclopropyl)phenyl)-1H-1,2,4-triazole, or a salt, solvate, isotopically labeled, enantiomer, diastereomer, and/or tautomer thereof. In certain embodiments, the compound is 5-(4-chlorophenyl)-1-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 5-(3,4-dimethylphenyl)-1-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 1-methyl-3-(pyrrolidin-1-ylmethyl)-5-(p-tolyl)-1H-1,2,4-triazole, or a salt, solvate, isotopically labeled, enantiomer, diastereomer, and/or tautomer thereof. In certain embodiments, the compound is 5-(4-cyclopropylphenyl)-1-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 5-(2,3-dihydro-1H-inden-5-yl)-1-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 5-(benzo[b]thiophen-6-yl)-1-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 1-methyl-3-(pyrrolidin-1-ylmethyl)-5-(2,3′,4′-trichloro-[1,1′-biphenyl]-4-yl)-1H-1,2,4-triazole, or a salt, solvate, isotopically labeled, enantiomer, diastereomer, and/or tautomer thereof. In certain embodiments, the compound is 5-(2-chlorobenzo[b]thiophen-6-yl)-1-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 5-(4-(tert-butyl)phenyl)-3-((3,3-dimethylpyrrolidin-1-yl)methyl)-1-methyl-1H-1,2,4-triazole, or a salt, solvate, isotopically labeled, enantiomer, diastereomer, and/or tautomer thereof. In certain embodiments, the compound is 2-((5-(4-(tert-butyl)phenyl)-1-methyl-1H-1,2,4-triazol-3-yl)methyl)-2-azaspiro[4.4]nonane, or a salt, solvate, isotopically labeled, enantiomer, diastereomer, and/or tautomer thereof. In certain embodiments, the compound is 5-((5-(4-(tert-butyl)phenyl)-1-methyl-1H-1,2,4-triazol-3-yl)methyl)-5-azaspiro[2.4]heptane, or a salt, solvate, isotopically labeled, enantiomer, diastereomer, and/or tautomer thereof. In certain embodiments, the compound is 6-((5-(4-(tert-butyl)phenyl)-1-methyl-1H-1,2,4-triazol-3-yl)methyl)-6-azaspiro[3.4]octane, or a salt, solvate, isotopically labeled, enantiomer, diastereomer, and/or tautomer thereof. In certain embodiments, the compound is 5-(4-(tert-butyl)phenyl)-1-ethyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 5-(4-(tert-butyl)phenyl)-1-isopropyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 5-(4-(tert-butyl)phenyl)-1-cyclopropyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 3-(4-(tert-butyl)phenyl)-1-ethyl-5-(pyrrolidin-1-ylmethyl)-1H-1,2,4-triazole, or a salt, solvate, isotopically labeled, enantiomer, diastereomer, and/or tautomer thereof. In certain embodiments, the compound is 3-(4-(tert-butyl)phenyl)-1-isopropyl-5-(pyrrolidin-1-ylmethyl)-1H-1,2,4-triazole, or a salt, solvate, isotopically labeled, enantiomer, diastereomer, and/or tautomer thereof. In certain embodiments, the compound is 1-(4-(tert-butyl)phenyl)-5-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 5-methyl-3-(pyrrolidin-1-ylmethyl)-1-(4-(1-(trifluoromethyl)cyclopropyl)phenyl)-1H-1,2,4-triazole, or a salt, solvate, isotopically labeled, enantiomer, diastereomer, and/or tautomer thereof. In certain embodiments, the compound is 1-(3,4-dichlorophenyl)-5-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 5-methyl-1-(3-methyl-4-(trifluoromethyl)phenyl)-3-(pyrrolidin-1-ylmethyl)-1H-1,2,4-triazole, or a salt, solvate, isotopically labeled, enantiomer, diastereomer, and/or tautomer thereof. In certain embodiments, the compound is, or a salt, solvate, isotopically labeled, enantiomer, diastereomer, and/or tautomer thereof. In certain embodiments, the compound is 5-([1,1′-biphenyl]-4-yl)-1-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 5-([1,1′-biphenyl]-3-yl)-1-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 1-methyl-5-(4-phenoxyphenyl)-3-(pyrrolidin-1-ylmethyl)-1H-1,2,4-triazole, or a salt, solvate, isotopically labeled, enantiomer, diastereomer, and/or tautomer thereof. In certain embodiments, the compound is 1-methyl-5-(3-phenoxyphenyl)-3-(pyrrolidin-1-ylmethyl)-1H-1,2,4-triazole, or a salt, solvate, isotopically labeled, enantiomer, diastereomer, and/or tautomer thereof. In certain embodiments, the compound is 5-(4-(benzyloxy)phenyl)-1-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 5-(3-(benzyloxy)phenyl)-1-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 5-(4-cyclohexylphenyl)-1-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 3-((5-(4-(tert-butyl)phenyl)-1-methyl-1H-1,2,4-triazol-3-yl)methyl)-3-azabicyclo[3.1.0]hexane, or a salt, solvate, isotopically labeled, enantiomer, diastereomer, and/or tautomer thereof. In certain embodiments, the compound is 2-((5-(4-(tert-butyl)phenyl)-1-methyl-1H-1,2,4-triazol-3-yl)methyl)octahydrocyclopenta[c]pyrrole, or a salt, solvate, isotopically labeled, enantiomer, diastereomer, and/or tautomer thereof. In certain embodiments, the compound is (1s, 4s)-7-((5-(4-(tert-butyl)phenyl)-1-methyl-1H-1,2,4-triazol-3-yl)methyl)-7-azabicyclo[2.2.1]heptane, or a salt, solvate, isotopically labeled, enantiomer, diastereomer, and/or tautomer thereof. In certain embodiments, the compound is 1-(benzo[b]thiophen-6-yl)-5-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 5-methyl-3-(pyrrolidin-1-ylmethyl)-1-(5,6,7,8-tetrahydronaphthalen-2-yl)-1H-1,2,4-triazole, or a salt, solvate, isotopically labeled, enantiomer, diastereomer, and/or tautomer thereof. In certain embodiments, the compound is 1-(4-cyclohexylphenyl)-5-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 1-([1,1′-biphenyl]-4-yl)-5-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 3-(4-(tert-butyl)phenyl)-1-methyl-5-(pyrrolidin-1-ylmethyl)-1H-1,2,4-triazole, or a salt, solvate, isotopically labeled, enantiomer, diastereomer, and/or tautomer thereof. In certain embodiments, the compound is 3-(4-(tert-butyl)phenyl)-1-cyclopropyl-5-(pyrrolidin-1-ylmethyl)-1H-1,2,4-triazole, or a salt, solvate, isotopically labeled, enantiomer, diastereomer, and/or tautomer thereof. In certain embodiments, the compound is 1-((5-(4-(tert-butyl)phenyl)-1-methyl-1H-1,2,4-triazol-3-yl)methyl)piperazine, or a salt, solvate, isotopically labeled, enantiomer, diastereomer, and/or tautomer thereof. In certain embodiments, the compound is N-((5-(4-(tert-butyl)phenyl)-1-methyl-1H-1,2,4-triazol-3-yl)methyl)-N-methylcyclopropanamine, or a salt, solvate, isotopically labeled, enantiomer, diastereomer, and/or tautomer thereof. In certain embodiments, the compound is 5-(2,3-dihydrobenzo[b]thiophen-6-yl)-1-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 5-(3-chlorophenyl)-1-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 1-methyl-3-(pyrrolidin-1-ylmethyl)-5-(3-(trifluoromethyl)phenyl)-1H-1,2,4-triazole, or a salt, solvate, isotopically labeled, enantiomer, diastereomer, and/or tautomer thereof. In certain embodiments, the compound is 1-methyl-3-(pyrrolidin-1-ylmethyl)-5-(m-tolyl)-1H-1,2,4-triazole, or a salt, solvate, isotopically labeled, enantiomer, diastereomer, and/or tautomer thereof. In certain embodiments, the compound is 5-(4-propylphenyl)-1-methyl-3-(pyrrolidin-1-ylmethyl)-1H-1,2,4-triazole, or a salt, solvate, isotopically labeled, enantiomer, diastereomer, and/or tautomer thereof. In certain embodiments, the compound is 5-(4-(tert-butyl)phenyl)-3-((3,3-dimethylpyrrolidin-1-yl)methyl)-1-ethyl-1H-1,2,4-triazole, or a salt, solvate, isotopically labeled, enantiomer, diastereomer, and/or tautomer thereof. In certain embodiments, the compound is 5-((5-(4-(tert-butyl)phenyl)-1-ethyl-1H-1,2,4-triazol-3-yl)methyl)-5-azaspiro[2.4]heptane, or a salt, solvate, isotopically labeled, enantiomer, diastereomer, and/or tautomer thereof. In certain embodiments, the compound is 6-((5-(4-(tert-butyl)phenyl)-1-ethyl-1H-1,2,4-triazol-3-yl)methyl)-6-azaspiro[3.4]octane, or a salt, solvate, isotopically labeled, enantiomer, diastereomer, and/or tautomer thereof. In certain embodiments, the compound is 2-((5-(4-(tert-butyl)phenyl)-1-ethyl-1H-1,2,4-triazol-3-yl)methyl)-2-azaspiro[4.4]nonane, or a salt, solvate, isotopically labeled, enantiomer, diastereomer, and/or tautomer thereof. In certain embodiments, the compound is 2-((5-(4-(tert-butyl)phenyl)-1-ethyl-1H-1,2,4-triazol-3-yl)methyl)octahydrocyclopenta[c]pyrrole, or a salt, solvate, isotopically labeled, enantiomer, diastereomer, and/or tautomer thereof. In certain embodiments, the compound is 5-(4-(tert-butyl)phenyl)-3-((3,3-dimethylpyrrolidin-1-yl)methyl)-1-isopropyl-1H-1,2,4-triazole, or a salt, solvate, isotopically labeled, enantiomer, diastereomer, and/or tautomer thereof. In certain embodiments, the compound is 5-((5-(4-(tert-butyl)phenyl)-1-isopropyl-1H-1,2,4-triazol-3-yl)methyl)-5-azaspiro[2.4]heptane, or a salt, solvate, isotopically labeled, enantiomer, diastereomer, and/or tautomer thereof. In certain embodiments, the compound is 6-((5-(4-(tert-butyl)phenyl)-1-isopropyl-1H-1,2,4-triazol-3-yl)methyl)-6-azaspiro[3.4]octane, or a salt, solvate, isotopically labeled, enantiomer, diastereomer, and/or tautomer thereof. In certain embodiments, the compound is 2-((5-(4-(tert-butyl)phenyl)-1-isopropyl-1H-1,2,4-triazol-3-yl)methyl)-2-azaspiro[4.4]nonane, or a salt, solvate, isotopically labeled, enantiomer, diastereomer, and/or tautomer thereof. In certain embodiments, the compound is 2-((5-(4-(tert-butyl)phenyl)-1-isopropyl-1H-1,2,4-triazol-3-yl)methyl)octahydrocyclopenta[c]pyrrole, or a salt, solvate, isotopically labeled, enantiomer, diastereomer, and/or tautomer thereof. In certain embodiments, the compound is 5-(4-(tert-butyl)phenyl)-1-cyclopropyl-3-((3,3-dimethylpyrrolidin-1-yl)methyl)-1H-1,2,4-triazole, or a salt, solvate, isotopically labeled, enantiomer, diastereomer, and/or tautomer thereof. In certain embodiments, the compound is 5-((5-(4-(tert-butyl)phenyl)-1-cyclopropyl-1H-1,2,4-triazol-3-yl)methyl)-5-azaspiro[2.4]heptane, or a salt, solvate, isotopically labeled, enantiomer, diastereomer, and/or tautomer thereof. In certain embodiments, the compound is 6-((5-(4-(tert-butyl)phenyl)-1-cyclopropyl-1H-1,2,4-triazol-3-yl)methyl)-6-azaspiro[3.4]octane, or a salt, solvate, isotopically labeled, enantiomer, diastereomer, and/or tautomer thereof. In certain embodiments, the compound is 2-((5-(4-(tert-butyl)phenyl)-1-cyclopropyl-1H-1,2,4-triazol-3-yl)methyl)-2-azaspiro[4.4]nonane, or a salt, solvate, isotopically labeled, enantiomer, diastereomer, and/or tautomer thereof. In certain embodiments, the compound is 2-((5-(4-(tert-butyl)phenyl)-1-cyclopropyl-1H-1,2,4-triazol-3-yl)methyl)octahydrocyclopenta[c]pyrrole, or a salt, solvate, isotopically labeled, enantiomer, diastereomer, and/or tautomer thereof.

The present invention further provides methods of preparing compounds of the present invention. Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field.

Compounds of the invention can be prepared, in a non-limiting embodiment, using the synthetic route outlined in Scheme I. Benzoic acid (i) can be coupled with hydrazine (ii) using standard coupling reagents, such as but not limited to HATU (1-[Bis(dimethylamino)methylene]-1H-1,2,3-triazolo[4,5-b]pyridinium 3-oxid hexafluorophosphate) or EDC (1-Ethyl-3-(3-dimethylaminopropyl)carbodiimide)/HOBt (Hydroxybenzotriazole) or EDC/PfP-OH (pentafluorophenol), in the presence of a tertiary amine, such as but not limited to triethylamine or diisopropylethylamine, in a solvent such as but limited to tetrahydrofuran or dichloromethane, to afford a mixture of acylhydrazines (iii) and (iv), which can be separated using standard separation methods, such as but not limited to recrystallization, precipitation, column separation, and/or extraction. Acylhydrazine (iii) can be coupled with compound (iv) under dehydrating conditions, for example, a mixture of toluene and acetic acid (10:1) at 100° C., to afford triazole (vi). Reduction of the ester in (vi) using, for example, a hydride, such as for example lithium aluminum hydride, affords alcohol (vii), which can be derivatized with an appropriate leaving group X to afford (viii). Non-limiting examples of X are Cl, Br, I, tosylate, mesylate, triflate, and the like. Reaction of (viii) with amine HNR¹R² affords a compound of Formula (I), wherein —X—Y— is —CH₂— (for example, n=0 and X═—CH₂—).

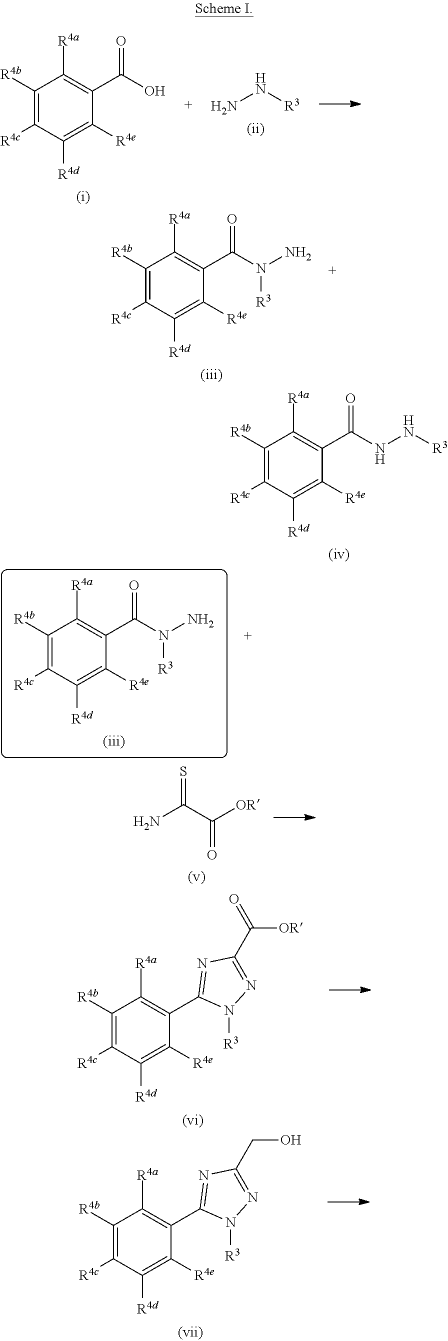

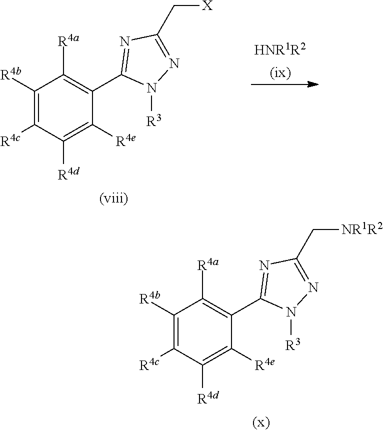

Non-limiting examples of amines (ix) contemplated within the invention follow:

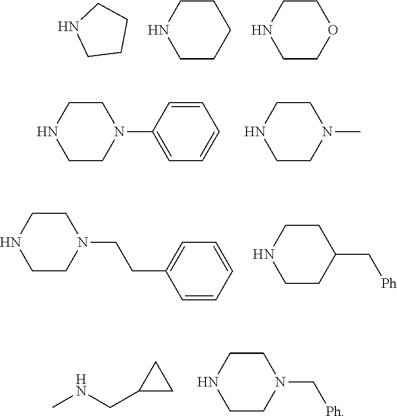

Compounds of the invention can be prepared, in a non-limiting embodiment, using the synthetic route outlined in Schemes I-IX. Groups R′ and R″ are shown for illustrative purposes only and represent groups described elsewhere herein, as it will be appreciated by one skilled in the art.

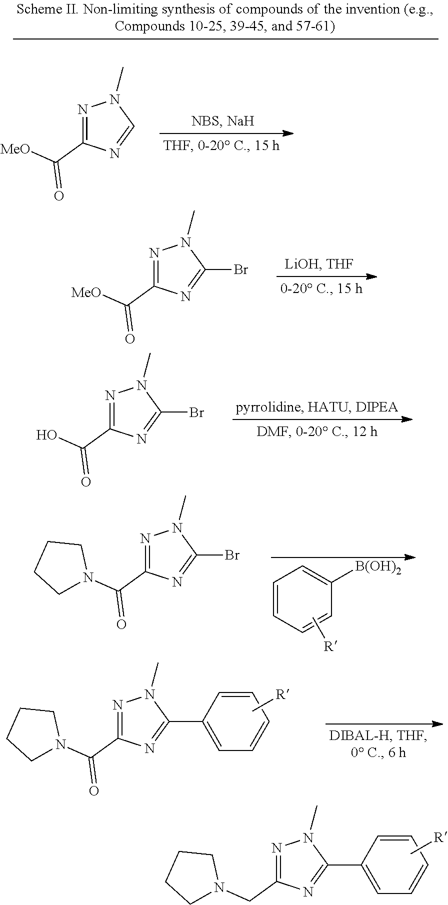

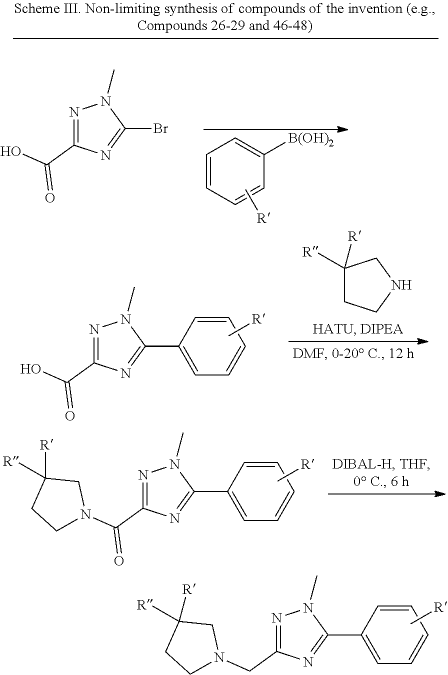

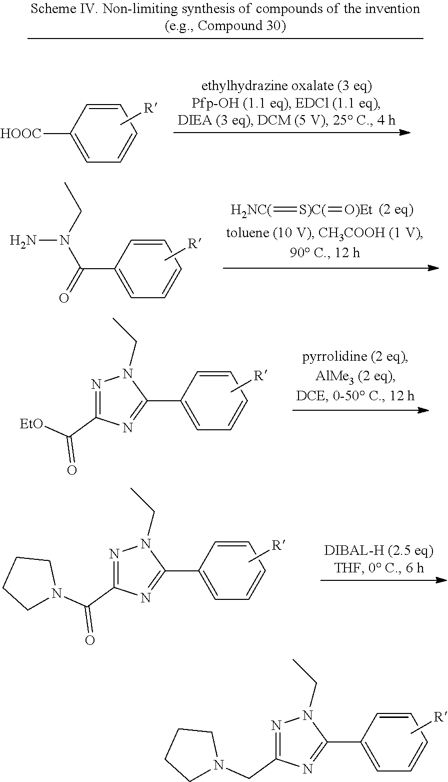

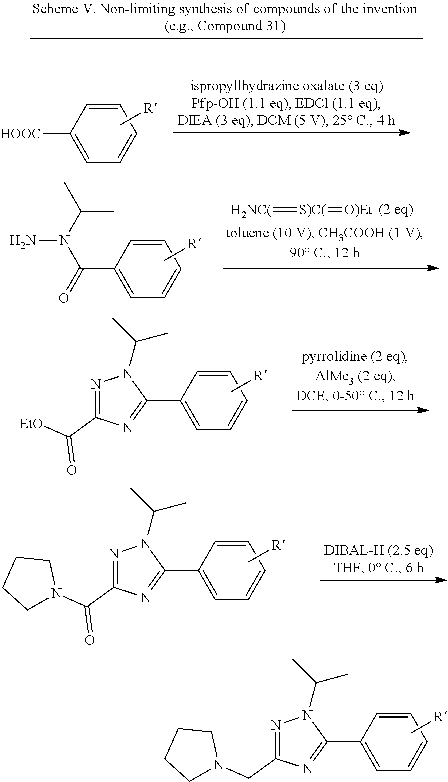

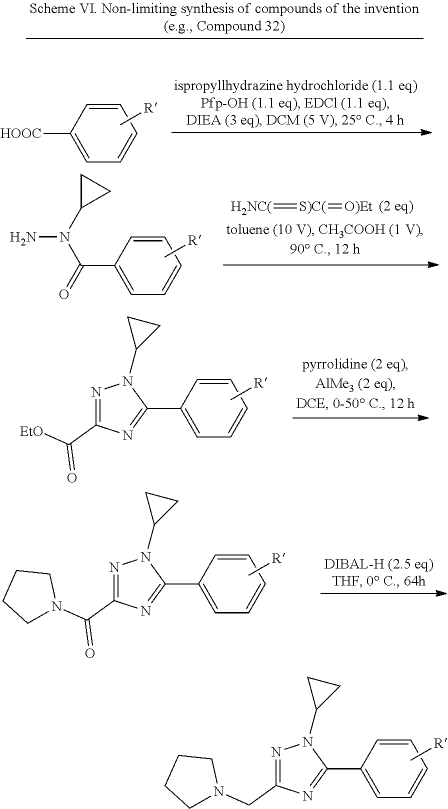

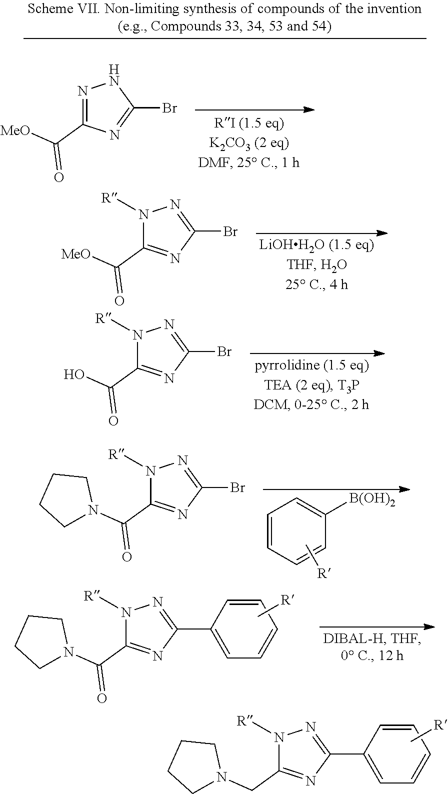

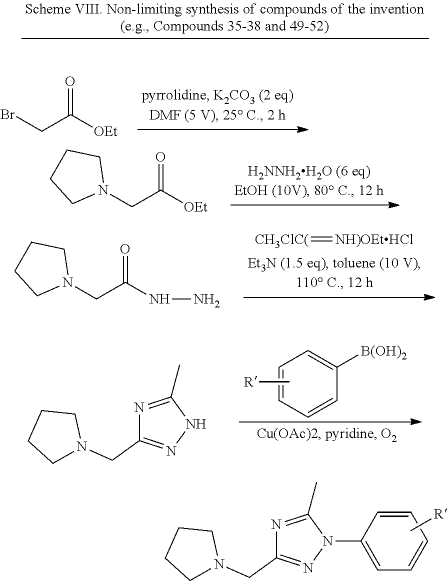

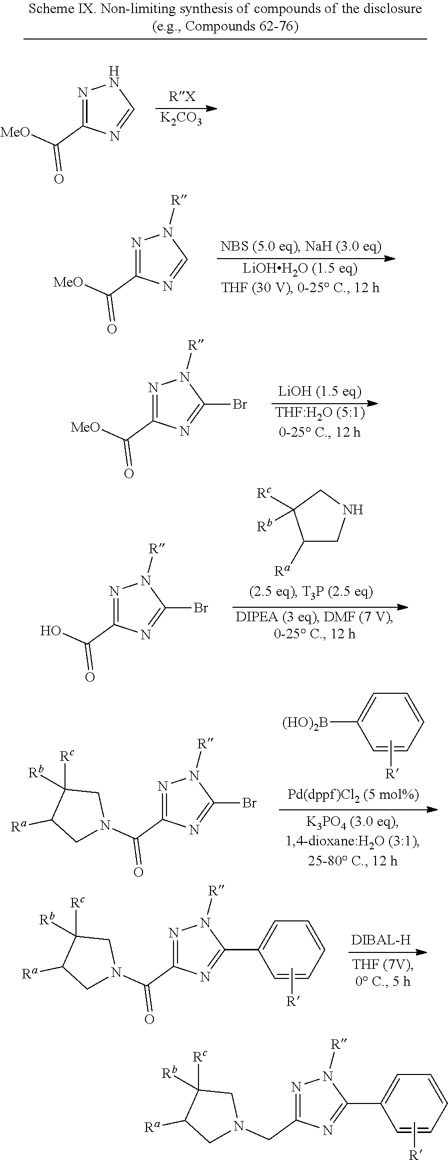

It is appreciated that where typical or preferred process conditions (i.e., reaction temperatures, times, mole ratios of reactants, solvents, pressures, and so forth) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high-performance liquid chromatography (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents that can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of the invention may possess one or more stereocenters, and each stereocenter may exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography. All possible stereochemical configurations of a given compound containing chiral center(s) are contemplated. All possible mixtures enriched with a particular enantiomer or diasteromer(s) are contemplated. All pure individual enantiomers or diastereomers are contemplated.

In certain embodiments, the compounds of the invention may exist as tautomers. “Tautomerization” is a form of isomerization involving the migration of a proton accompanied by changes in bond order, often the interchange of a single bond with an adjacent double bond. Where tautomerization is possible (e.g., in solution), a chemical equilibrium of tautomers can be reached. One well known example of tautomerization is between a ketone and its corresponding enol. Heterocycles may form tautomers such as the interconversion of pyrrolidinone and hydroxypyrrole. All tautomers are included within the scope of the compounds presented herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed invention. The compounds of the invention may contain any of the substituents, or combinations of substituents, provided herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

Compounds of the invention can in certain embodiments form acids or bases. In certain embodiments, the invention contemplates acid addition salts. In other embodiments, the invention contemplates base addition salts. In yet other embodiments, the invention contemplates pharmaceutically acceptable acid addition salts. In yet other embodiments, the invention contemplates pharmaceutically acceptable base addition salts. Pharmaceutically acceptable salts refer to salts of those bases or acids that are not toxic or otherwise biologically undesirable.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds of the invention include, for example, metallic salts including alkali metal, alkaline earth metal and transition metal salts such as, for example, calcium, magnesium, potassium, sodium, lithium and copper, iron and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

### Methods

The invention provides methods of binding to at least one sigma receptor selected from the group consisting of S1R and S2R in a cell, tissue, and/or subject.

The invention further provides methods of modulating activity of at least one sigma receptor selected from the group consisting of S1R and S2R in a cell, tissue, and/or subject.

The invention further provides methods of increasing activity of at least one sigma receptor selected from the group consisting of S1R and S2R in a cell, tissue, and/or subject.

The invention further provides methods of reducing activity of at least one sigma receptor selected from the group consisting of S1R and S2R in a cell, tissue, and/or subject.

The invention further provides methods of modulating (i.e., increasing or decreasing) opioid receptor signaling in a cell, tissue, and/or subject.

The invention further provides methods of modulating N-methyl-D-aspartate (NMDA) receptor activity in a cell, tissue, and/or subject.

The invention further provides methods of treating, preventing, and/or ameliorating pain in a subject. In certain embodiments, the pain comprises chronic pain. In certain embodiments, the pain comprises acute pain. In other embodiments, the pain comprises neuropathic pain. In yet other embodiments, the pain comprises nociceptive pain. In yet other embodiments, the pain comprises hyperalgesia. In yet other embodiments, the pain comprises allodynia.

In certain embodiments, the methods of the invention diminish pain without producing respiratory depression. In certain embodiments, the methods of the invention diminish pain without producing constipation. In certain embodiments, the methods of the invention diminish pain without producing nauseas. In certain embodiments, the methods of the invention diminish pain without producing significant (or any at all) tolerance. In certain embodiments, the methods of the invention diminish pain without producing significant (or any at all) tachyphylaxis. In certain embodiments, the methods of the invention diminish pain without producing emesis. In certain embodiments, the methods of the invention diminish pain without producing withdrawal adverse effects. In certain embodiments, the methods of the invention diminish pain without producing dependence. In certain embodiments, the methods of the invention diminish pain without producing one or more of the following phenomena: nausea, emesis, constipation, respiratory depression, tolerance, tachyphylaxis, dependence and/or addiction. In certain embodiments, the methods of the invention do not cause at least one side effect selected from the group consisting of addiction, respiratory depression, euphoria, and constipation in the subject.

The invention further provides methods of promoting neuroprotection in a subject. The invention further provides methods of inhibiting, treating, and/or preventing neurological and neurodegenerative diseases, such as but not limited to Alzheimer's disease-type dementia, motorneuron disease, and/or amyotrophic lateral sclerosis (ALS). The invention further provides methods of improving learning and/or memory in a subject.

The invention further provides methods of inhibiting, treating, and/or preventing cancer and any related proliferative diseases.

The invention further provides methods of inhibiting, treating, and/or preventing substance abuse disorders in a subject. The invention further provides methods of inhibiting, treating, and/or preventing depressive disorders in a subject. The invention further provides methods of inhibiting, treating, and/or preventing ocular disorder and/or glaucoma in a subject.

In certain embodiments, the method comprises administering to the subject a therapeutically effective amount of a compound of the invention. In other embodiments, the method comprises contacting a compound of the invention with at least one sigma receptor selected from the group consisting of S1R and S2R in the cell, tissue, and/or subject. In yet other embodiments, the method comprises contacting a compound of the invention with S1R in the cell, tissue, and/or subject. In other yet embodiments, the method comprises contacting a compound of the invention with S2R in the cell, tissue, and/or subject.

In certain embodiments, the compound of the invention is the only therapeutically active agent administered to the subject. In other embodiments, the compound of the invention is the only therapeutically active agent administered to the subject in an amount sufficient to produce the desired therapeutic effect.

In certain embodiments, the subject is further administered an opioid agent. In other embodiments, administration of the compound and the opioid agent to the subject allows for a lower amount of the opioid agent to be administered than if the subject is not administered the compound. In yet other embodiments, administration of the compound and the opioid agent to the subject allows for use of an amount of the opioid agent that does not cause any significant side effect associated with opioid agent use.

### Combination Therapies

In one aspect, the compounds of the invention are useful within the methods of the invention in combination with one or more additional agents useful for treating a disease or disorder contemplated herein. These additional agents may comprise compounds or compositions identified herein, or compounds (e.g., commercially available compounds) known to treat, prevent, or reduce the symptoms of a disease or disorder contemplated herein.

A synergistic effect may be calculated, for example, using suitable methods such as, for example, the Sigmoid-Emax equation (Holford & Scheiner, 1981, Clin. Pharmacokinet. 6:429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to elsewhere herein may be applied to experimental data to generate a corresponding graph to aid in assessing the effects of the drug combination. The corresponding graphs associated with the equations referred to elsewhere herein are the concentration-effect curve, isobologram curve and combination index curve, respectively.

### Administration/Dosage/Formulations

The invention also encompasses pharmaceutical compositions and methods of their use. These pharmaceutical compositions may comprise an active ingredient (which can be one or more compounds of the invention, or pharmaceutically acceptable salts thereof) optionally in combination with one or more pharmaceutically acceptable agents. The compositions set forth herein can be used alone or in combination with additional compounds to produce additive, complementary, or synergistic effects.

The regimen of administration may affect what constitutes an effective amount. The therapeutic formulations may be administered to the subject either prior to or after the onset of a disease or disorder contemplated herein. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated herein. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated herein. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder contemplated herein.

In certain embodiments, the compositions of the invention are formulated using one or more pharmaceutically acceptable excipients or carriers. In certain embodiments, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin.

In certain embodiments, the compositions of the invention are administered to the patient in dosages that range from one to five times per day or more. In other embodiments, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

In certain embodiments, the dose of a compound of the invention is from about 1 mg and about 2,500 mg.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated herein.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycolate); or wetting agents (e.g., sodium lauryl sulfate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In certain embodiments, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Dosing

The therapeutically effective amount or dose of a compound of the present invention depends on the age, sex and weight of the patient, the current medical condition of the patient and the progression of a disease or disorder contemplated herein in the patient being treated. The skilled artisan is able to determine appropriate dosages depending on these and other factors.

A suitable dose of a compound of the present invention may be in the range of from about 0.001 mg to about 5,000 mg per day, such as from about 0.01 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Materials and Methods

### Binding Affinity for SIR and S2R in Radiolabeled Assays of Guinea Pig Brain

The radiolabeled binding assay of the S1R and S2R were performed using Guinea pig whole brain according to the protocol previously reported (Ganapathy, et al., 1999, J. Pharmacol. Exp. Ther. 289:251-60). The S1R binding assay was conducted in duplicate on membrane preparations that had been re-suspended in 50 mM Tris-HCl at pH=7.4 using [³H]-pentazocine as the radioligand. Following 1 h incubation at 25° C., the binding assay was terminated by addition of cold buffer. The mixture was then filtered through Whatman GF/B filters and washed with cold buffer. Radioactivity was identified using the liquid scintillation counter. Non-specific binding for the S1R was measured in the presence of 10 μM unlabeled (+)-pentazocine.

The S2R binding assay was conducted in duplicate on membrane preparations that had been re-suspended in 50 mM Tris-HCl at pH=7.4 using [³H]-DTG as the radioligand. Following 1 h incubation at 25° C., the binding assay was terminated by addition of cold buffer. The mixture was then filtered through Whatman GF/B filters and washed with cold buffer. Radioactivity was identified using the liquid scintillation counter. Non-specific binding for the S2R was measured in the presence of unlabeled haloperidol.

Receptor binding data were analyzed by nonlinear regression of saturation and competition curves using the GraphPad Prism 7.0 software (GraphPad Software, La Jolla, Calif.).

### Binding Affinity for SIR and S2R in Radiolabeled Assays of Human Jurkat Cell Membranes

The radiolabeled binding assay of the human S1R and S2R were performed using human jurkat cell membranes according to the protocol previously reported (Ganapathy, et al., 1999, J. Pharmacol. Exp. Ther. 289:251-60).

The binding affinity for the human SlR was experimentally determined in human jurkat cell membranes using 15.0 nM [³H]-pentazocine as the radioligand. Membranes were incubated with ligands in 50 mM Tris-HCl at pH 8.0 for 2 hours at 37° C. The binding process was terminated by addition of cold buffer. Then, the mixture was filtered and washed with cold buffer. Radioactivity was identified using the TopCount NTX liquid scintillation counter (PerkinElmer, Waltham, Mass.). Non-specific binding for the S1R was measured in the presence of 10 μM unlabeled haloperidol.

The binding affinity for human S2R was performed using Jurkat cell line Clone E6-1 cells with 0.025 μM [³H] DTG as radioligand. The non-specific binding was performed with 10 μM haloperidol. The cells were incubated in RPMI 1640 Medium with 10% FBS, 100 U/ml penicillin and 100 mg/ml streptomycin. After incubation, the cells were suspended in 5 mM potassium phosphate buffer (pH=7.6). The membrane protein concentration was controlled at 5 mg/mL. The collected membrane protein was incubated with ligands in potassium phosphate buffer for 1 hour at room temperature and terminated by adding ice-cold buffer. Then the mixture is filtered with 0.7 μm syringe filter and washed three times. The radioactivity is determined by liquid scintillation spectrometry.

### Example 1: Binding Affinity to the S1R and S2R

Radiolabeled binding assays were conducted using two different species. Compounds 1-9 were assayed using guinea pig brain membranes for the binding assays. Compounds 10-76 were assayed using human brain membranes for the binding assays. Results are summarized in Table 1 and Table 2.

TABLE 1
Binding Affinity to the S1R at 100 nM

| | | S1R % binding at |
|---|---|---|
| Cmpd | Substance | 100 nM |
| 1 | 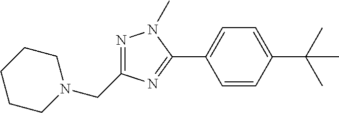 [SMILES: Cn1nc(CN2CCCCC2)nc1-c1ccc(C(C)(C)C)cc1] | 94 |
| | 1-((5-(4-(tert-butyl)phenyl)-1-methyl-1H-1,2,4-triazol-3- | |
| | yl)methyl)piperidine | |
| 2 | 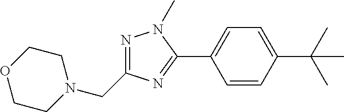 [SMILES: Cn1nc(CN2CCOCC2)nc1-c1ccc(C(C)(C)C)cc1] | 52 |
| | 4-((5-(4-(tert-butyl)phenyl)-1-methyl-1H-1,2,4-triazol-3- | |
| | yl)methyl)morpholine | |
| 3 | 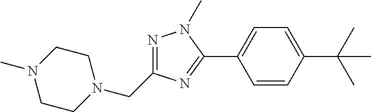 [SMILES: CN1CCN(Cc2nc(-c3ccc(C(C)(C)C)cc3)n(C)n2)CC1] | 32 |
| | 1-((5-(4-(tert-butyl)phenyl)-1-methyl-1H-1,2,4-triazol-3- | |
| | yl)methyl)-4-methylpiperazine | |
| 4 | 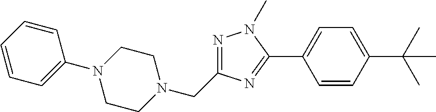 [SMILES: Cn1nc(CN2CCN(c3ccccc3)CC2)nc1-c1ccc(C(C)(C)C)cc1] | 77 |
| | 1-((5-(4-(tert-butyl)phenyl)-1-methyl-1H-1,2,4-triazol-3- | |
| | yl)methyl)-4-phenylpiperazine | |
| 5 | 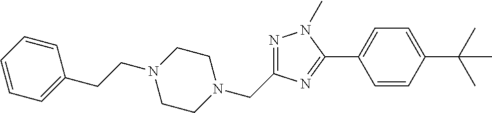 [SMILES: Cn1nc(CN2CCN(CCc3ccccc3)CC2)nc1-c1ccc(C(C)(C)C)cc1] | 85 |
| | 1-((5-(4-(tert-butyl)phenyl)-1-methyl-1H-1,2,4-triazol-3- | |
| | yl)methyl)-4-phenethylpiperazine | |
| 6 | 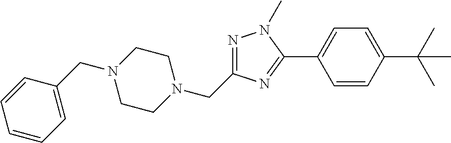 [SMILES: Cn1nc(CN2CCN(Cc3ccccc3)CC2)nc1-c1ccc(C(C)(C)C)cc1] | 91 |
| | 1-benzyl-4-((5-(4-(tert-butyl)phenyl)-1-methyl-1H-1,2,4- | |
| | triazol-3-yl)methyl)piperazine | |
| 7 | 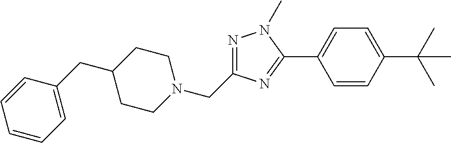 [SMILES: Cn1nc(CN2CCC(Cc3ccccc3)CC2)nc1-c1ccc(C(C)(C)C)cc1] | 95 |
| | 4-benzyl-1-((5-(4-(tert-butyl)phenyl)-1-methyl-1H-1,2,4- | |
| | triazol-3-yl)methyl)piperidine | |
| 8 | 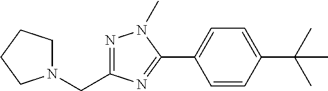 [SMILES: Cn1nc(CN2CCCC2)nc1-c1ccc(C(C)(C)C)cc1] | 95 |
| | 5-(4-(tert-butyl)phenyl)-1-methyl-3-(pyrrolidin-1- | |
| | ylmethyl)-1H-1,2,4-triazole | |
| 9 | 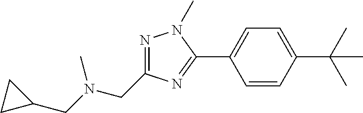 [SMILES: CN(Cc1nc(-c2ccc(C(C)(C)C)cc2)n(C)n1)CC1CC1] | 98 |
| | 1-(5-(4-(tert-butyl)phenyl)-1-methyl-1H-1,2,4-triazol-3- | |
| | yl)-N-(cyclopropylmethyl)-N-methylmethanamine | |
| 10 | 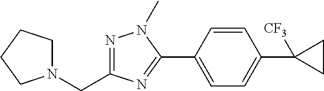 [SMILES: Cn1nc(CN2CCCC2)nc1-c1ccc(C2(C(F)(F)F)CC2)cc1] | 77 |
| | 1-methyl-3-(pyrrolidin-1-ylmethyl)-5-(4-(1- | |
| | (trifluoromethyl)cyclopropyl)phenyl)-1H-1,2,4-triazole | |
| 11 | 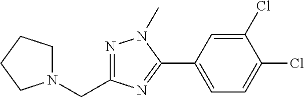 [SMILES: Cn1nc(CN2CCCC2)nc1-c1ccc(Cl)c(Cl)c1] | 68 |
| | 5-(3,4-dichlorophenyl)-1-methyl-3-(pyrrolidin-1- | |
| | ylmethyl)-1H-1,2,4-triazole | |
| 12 | 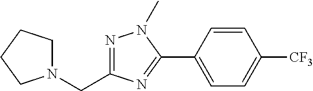 [SMILES: Cn1nc(CN2CCCC2)nc1-c1ccc(C(F)(F)F)cc1] | 55 |
| | 1-methyl-3-(pyrrolidin-1-ylmethyl)-5-(4- | |
| | (trifluoromethyl)phenyl)-1H-1,2,4-triazole | |
| 13 | 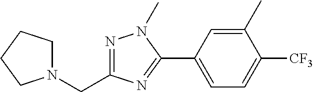 [SMILES: Cc1cc(-c2nc(CN3CCCC3)nn2C)ccc1C(F)(F)F] | 83 |
| | 1-methyl-5-(3-methyl-4-(trifluoromethyl)phenyl)-3- | |
| | (pyrrolidin-1-ylmethyl)-1H-1,2,4-triazole | |
| 14 | 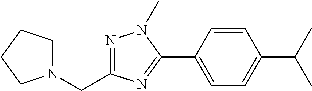 [SMILES: CC(C)c1ccc(-c2nc(CN3CCCC3)nn2C)cc1] | 53 |
| | 5-(4-isopropylphenyl)-1-methyl-3-(pyrrolidin-1-ylmethyl)- | |
| | 1H-1,2,4-triazole | |
| 15 | 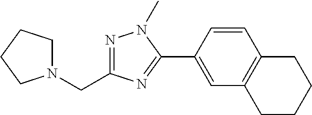 [SMILES: Cn1nc(CN2CCCC2)nc1-c1ccc2c(c1)CCCC2] | 65 |
| | 1-methyl-3-(pyrrolidin-1-ylmethyl)-5-(5,6,7,8- | |
| | tetrahydronaphthalen-2-yl)-1H-1,2,4-triazole | |
| 16 | 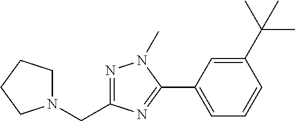 [SMILES: Cn1nc(CN2CCCC2)nc1-c1cccc(C(C)(C)C)c1] | 58 |
| | 5-(3-(tert-butyl)phenyl)-1-methyl-3-(pyrrolidin-1- | |
| | ylmethyl)-1H-1,2,4-triazole | |
| 17 | 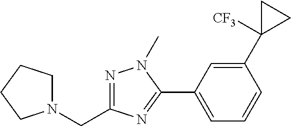 [SMILES: Cn1nc(CN2CCCC2)nc1-c1cccc(C2(C(F)(F)F)CC2)c1] | 64 |
| | 1-methyl-3-(pyrrolidin-1-ylmethyl)-5-(3-(1- | |
| | (trifluoromethyl)cyclopropyl)phenyl)-1H-1,2,4-triazole | |
| 18 | 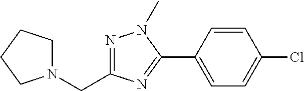 [SMILES: Cn1nc(CN2CCCC2)nc1-c1ccc(Cl)cc1] | 31 |
| | 5-(4-chlorophenyl)-1-methyl-3-(pyrrolidin-1-ylmethyl)- | |
| | 1H-1,2,4-triazole | |
| 19 | 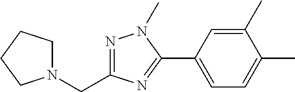 [SMILES: Cc1ccc(-c2nc(CN3CCCC3)nn2C)cc1C] | 28 |
| | 5-(3,4-dimethylphenyl)-1-methyl-3-(pyrrolidin-1- | |
| | ylmethyl)-1H-1,2,4-triazole | |
| 20 | 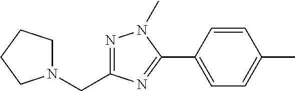 [SMILES: Cc1ccc(-c2nc(CN3CCCC3)nn2C)cc1] | 3 |
| | 1-methyl-3-(pyrrolidin-1-ylmethyl)-5-(p-tolyl)-1H-1,2,4- | |
| | triazole | |
| 21 | 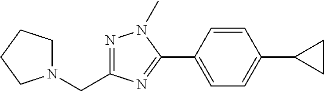 [SMILES: Cn1nc(CN2CCCC2)nc1-c1ccc(C2CC2)cc1] | 30 |
| | 5-(4-cyclopropylphenyl)-1-methyl-3-(pyrrolidin-1- | |
| | ylmethyl)-1H-1,2,4-triazole | |
| 22 | 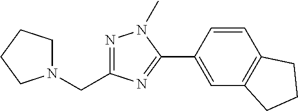 [SMILES: Cn1nc(CN2CCCC2)nc1-c1ccc2c(c1)CCC2] | 37 |
| | 5-(2,3-dihydro-1H-inden-5-yl)-1-methyl-3-(pyrrolidin-1- | |
| | ylmethyl)-1H-1,2,4-triazole | |
| 23 | 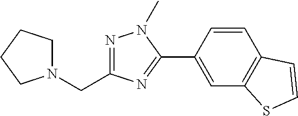 [SMILES: Cn1nc(CN2CCCC2)nc1-c1ccc2ccsc2c1] | 57 |
| | 5-(benzo[b]thiophen-6-yl)-1-methyl-3-(pyrrolidin-1- | |
| | ylmethyl)-1H-1,2,4-triazole | |
| 24 | 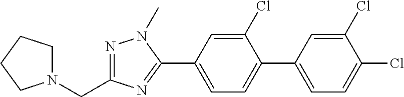 [SMILES: Cn1nc(CN2CCCC2)nc1-c1ccc(-c2ccc(Cl)c(Cl)c2)c(Cl)c1] | 43 |
| | 1-methyl-3-(pyrrolidin-1-ylmethyl)-5-(2,3′,4′-trichloro- | |
| | [1,1′-biphenyl]-4-yl)-1H-1,2,4-triazole | |
| 25 | 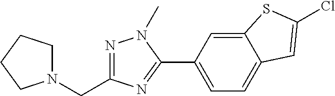 [SMILES: Cn1nc(CN2CCCC2)nc1-c1ccc2cc(Cl)sc2c1] | 69 |
| | 5-(2-chlorobenzo[b]thiophen-6-yl)-1-methyl-3-(pyrrolidin- | |
| | 1-ylmethyl)-1H-1,2,4-triazole | |
| 26 | 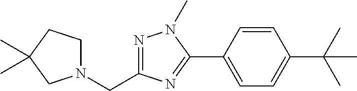 [SMILES: Cn1nc(CN2CCC(C)(C)C2)nc1-c1ccc(C(C)(C)C)cc1] | 80 |
| | 5-(4-(tert-butyl)phenyl)-3-((3,3-dimethylpyrrolidin-1- | |
| | yl)methyl)-1-methyl-1H-1,2,4-triazole | |
| 27 | 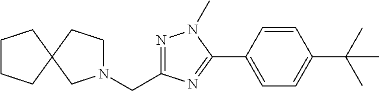 [SMILES: Cn1nc(CN2CCC3(CCCC3)C2)nc1-c1ccc(C(C)(C)C)cc1] | 111 |
| | 2-((5-(4-(tert-butyl)phenyl)-1-methyl-1H-1,2,4-triazol-3- | |
| | yl)methyl)-2-azaspiro[4.4]nonane | |
| 28 | 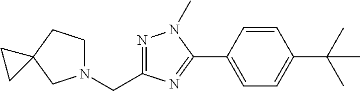 [SMILES: Cn1nc(CN2CCC3(CC3)C2)nc1-c1ccc(C(C)(C)C)cc1] | 98 |
| | 5-((5-(4-(tert-butyl)phenyl)-1-methyl-1H-1,2,4-triazol-3- | |
| | yl)methyl)-5-azaspiro[2.4]heptane | |
| 29 | 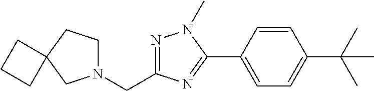 [SMILES: Cn1nc(CN2CCC3(CCC3)C2)nc1-c1ccc(C(C)(C)C)cc1] | 100 |
| | 6-((5-(4-(tert-butyl)phenyl)-1-methyl-1H-1,2,4-triazol-3- | |
| | yl)methyl)-6-azaspiro[3.4]octane | |
| 30 | 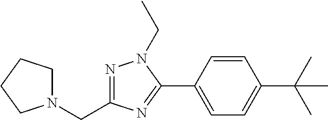 [SMILES: CCn1nc(CN2CCCC2)nc1-c1ccc(C(C)(C)C)cc1] | 100 |
| | 5-(4-(tert-butyl)phenyl)-1-ethyl-3-(pyrrolidin-1-ylmethyl)- | |
| | 1H-1,2,4-triazole | |
| 31 | 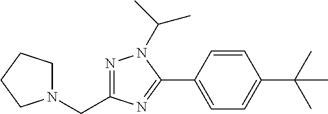 [SMILES: CC(C)n1nc(CN2CCCC2)nc1-c1ccc(C(C)(C)C)cc1] | 98 |
| | 5-(4-(tert-butyl)phenyl)-1-isopropyl-3-(pyrrolidin-1- | |
| | ylmethyl)-1H-1,2,4-triazole | |
| 32 | 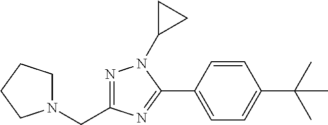 [SMILES: CC(C)(C)c1ccc(-c2nc(CN3CCCC3)nn2C2CC2)cc1] | 92 |
| | 5-(4-(tert-butyl)phenyl)-1-cyclopropyl-3-(pyrrolidin-1- | |
| | ylmethyl)-1H-1,2,4-triazole | |
| 33 | 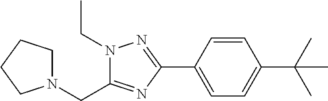 [SMILES: CCn1nc(-c2ccc(C(C)(C)C)cc2)nc1CN1CCCC1] | 17 |
| | 3-(4-(tert-butyl)phenyl)-1-ethyl-5-(pyrrolidin-1-ylmethyl)- | |
| | 1H-1,2,4-triazole | |
| 34 | 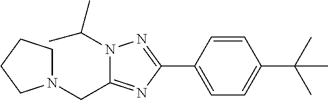 [SMILES: CC(C)n1nc(-c2ccc(C(C)(C)C)cc2)nc1CN1CCCC1] | 13 |
| | 3-(4-(tert-butyl)phenyl)-1-isopropyl-5-(pyrrolidin-1- | |
| | ylmethyl)-1H-1,2,4-triazole | |
| 35 | 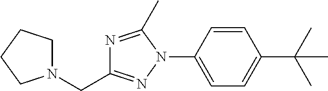 [SMILES: Cc1nc(CN2CCCC2)nn1-c1ccc(C(C)(C)C)cc1] | 94 |
| | 1-(4-(tert-butyl)phenyl)-5-methyl-3-(pyrrolidin-1- | |
| | ylmethyl)-1H-1,2,4-triazole | |
| 36 | 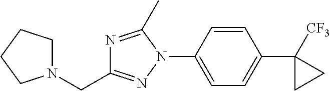 [SMILES: Cc1nc(CN2CCCC2)nn1-c1ccc(C2(C(F)(F)F)CC2)cc1] | 74 |
| | 5-methyl-3-(pyrrolidin-1-ylmethyl)-1-(4-(1- | |
| | (trifluoromethyl)cyclopropyl)phenyl)-1H-1,2,4-triazole | |
| 37 | 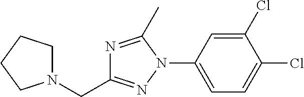 [SMILES: Cc1nc(CN2CCCC2)nn1-c1ccc(Cl)c(Cl)c1] | 72 |
| | 1-(3,4-dichlorophenyl)-5-methyl-3-(pyrrolidin-1- | |
| | ylmethyl)-1H-1,2,4-triazole | |
| 38 | 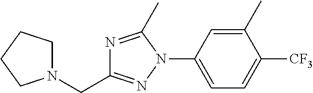 [SMILES: Cc1cc(-n2nc(CN3CCCC3)nc2C)ccc1C(F)(F)F] | 76 |
| | 5-methyl-1-(3-methyl-4-(trifluoromethyl)phenyl)-3- | |
| | (pyrrolidin-1-ylmethyl)-1H-1,2,4-triazole | |
| 39 | 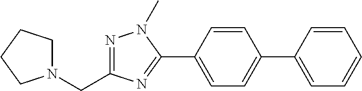 [SMILES: Cn1nc(CN2CCCC2)nc1-c1ccc(-c2ccccc2)cc1] | 54 |
| | 5-([1,1′-biphenyl]-4-yl)-1-methyl-3-(pyrrolidin-1- | |
| | ylmethyl)-1H-1,2,4-triazole | |
| 40 | 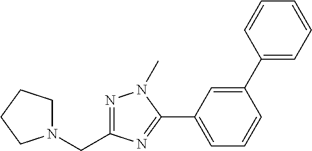 [SMILES: Cn1nc(CN2CCCC2)nc1-c1cccc(-c2ccccc2)c1] | 40 |
| | 5-([1,1′-biphenyl]-3-yl)-1-methyl-3-(pyrrolidin-1- | |
| | ylmethyl)-1H-1,2,4-triazole | |
| 41 | 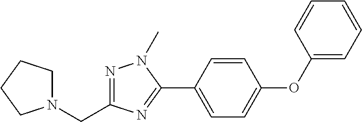 [SMILES: Cn1nc(CN2CCCC2)nc1-c1ccc(Oc2ccccc2)cc1] | −21 |
| | 1-methyl-5-(4-phenoxyphenyl)-3-(pyrrolidin-1-ylmethyl)- | |
| | 1H-1,2,4-triazole | |
| 42 | 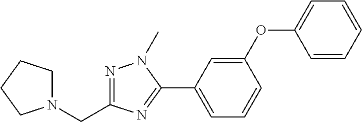 [SMILES: Cn1nc(CN2CCCC2)nc1-c1cccc(Oc2ccccc2)c1] | 21 |
| | 1-methyl-5-(3-phenoxyphenyl)-3-(pyrrolidin-1-ylmethyl)- | |
| | 1H-1,2,4-triazole | |
| 43 | 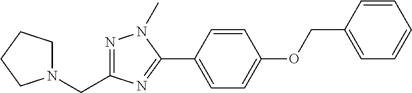 [SMILES: Cn1nc(CN2CCCC2)nc1-c1ccc(OCc2ccccc2)cc1] | 6 |
| | 5-(4-(benzyloxy)phenyl)-1-methyl-3-(pyrrolidin-1- | |
| | ylmethyl)-1H-1,2,4-triazole | |
| 44 | 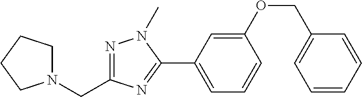 [SMILES: Cn1nc(CN2CCCC2)nc1-c1cccc(OCc2ccccc2)c1] | 9 |
| | 5-(3-(benzyloxy)phenyl)-1-methyl-3-(pyrrolidin-1- | |
| | ylmethyl)-1H-1,2,4-triazole | |
| 45 | 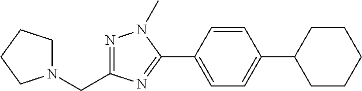 [SMILES: Cn1nc(CN2CCCC2)nc1-c1ccc(C2CCCCC2)cc1] | 64 |
| | 5-(4-cyclohexylphenyl)-1-methyl-3-(pyrrolidin-1- | |
| | ylmethyl)-1H-1,2,4-triazole | |
| 46 | 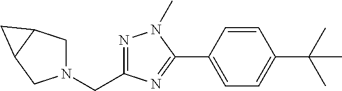 [SMILES: Cn1nc(CN2CC3CC3C2)nc1-c1ccc(C(C)(C)C)cc1] | 74 |
| | 3-((5-(4-(tert-butyl)phenyl)-1-methyl-1H-1,2,4-triazol-3- | |
| | yl)methyl)-3-azabicyclo[3.1.0]hexane | |
| 47 | 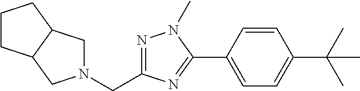 [SMILES: Cn1nc(CN2CC3CCCC3C2)nc1-c1ccc(C(C)(C)C)cc1] | 106 |
| | 2-((5-(4-(tert-butyl)phenyl)-1-methyl-1H-1,2,4-triazol-3- | |
| | yl)methyl)octahydrocyclopenta[c]pyrrole | |
| 48 | 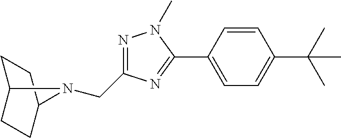 [SMILES: Cn1nc(CN2C3CCC2CC3)nc1-c1ccc(C(C)(C)C)cc1] | 91 |
| | (1s,4s)-7-((5-(4-(tert-butyl)phenyl)-1-methyl-1H-1,2,4- | |
| | triazol-3-yl)methyl)-7-azabicyclo[2.2.1]heptane | |
| 49 | 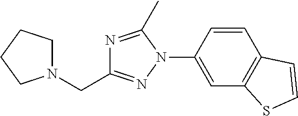 [SMILES: Cc1nc(CN2CCCC2)nn1-c1ccc2ccsc2c1] | 51 |
| | 1-(benzo[b]thiophen-6-yl)-5-methyl-3-(pyrrolidin-1- | |
| | ylmethyl)-1H-1,2,4-triazole | |
| 50 | 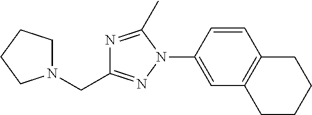 [SMILES: Cc1nc(CN2CCCC2)nn1-c1ccc2c(c1)CCCC2] | 51 |
| | 5-methyl-3-(pyrrolidin-1-ylmethyl)-1-(5,6,7,8- | |
| | tetrahydronaphthalen-2-yl)-1H-1,2,4-triazole | |
| 51 | 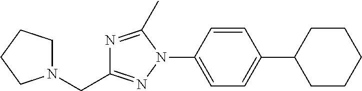 [SMILES: Cc1nc(CN2CCCC2)nn1-c1ccc(C2CCCCC2)cc1] | 69 |
| | 1-(4-cyclohexylphenyl)-5-methyl-3-(pyrrolidin-1- | |
| | ylmethyl)-1H-1,2,4-triazole | |
| 52 | 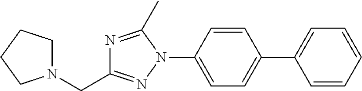 [SMILES: Cc1nc(CN2CCCC2)nn1-c1ccc(-c2ccccc2)cc1] | 71 |
| | 1-([1,1′-biphenyl]-4-yl)-5-methyl-3-(pyrrolidin-1- | |
| | ylmethyl)-1H-1,2,4-triazole | |
| 53 | 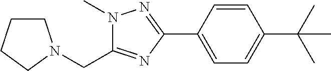 [SMILES: Cn1nc(-c2ccc(C(C)(C)C)cc2)nc1CN1CCCC1] | 34 |
| | 3-(4-(tert-butyl)phenyl)-1-methyl-5-(pyrrolidin-1- | |
| | ylmethyl)-1H-1,2,4-triazole | |
| 54 | 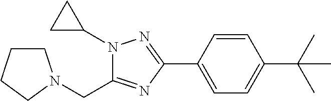 [SMILES: CC(C)(C)c1ccc(-c2nc(CN3CCCC3)n(C3CC3)n2)cc1] | 29 |
| | 3-(4-(tert-butyl)phenyl)-1-cyclopropyl-5-(pyrrolidin-1- | |
| | ylmethyl)-1H-1,2,4-triazole | |
| 55 | 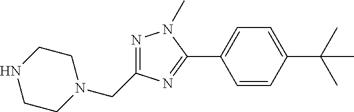 [SMILES: Cn1nc(CN2CCNCC2)nc1-c1ccc(C(C)(C)C)cc1] | 40 |
| | 1-((5-(4-(tert-butyl)phenyl)-1-methyl-1H-1,2,4-triazol-3- | |
| | yl)methyl)piperazine | |
| 56 | 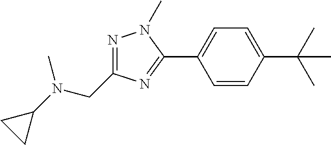 [SMILES: CN(Cc1nc(-c2ccc(C(C)(C)C)cc2)n(C)n1)C1CC1] | 55 |
| | N-((5-(4-(tert-butyl)phenyl)-1-methyl-1H-1,2,4-triazol-3- | |
| | yl)methyl)-N-methylcyclopropanamine | |
| 57 | 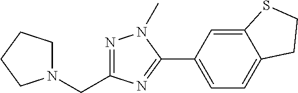 [SMILES: Cn1nc(CN2CCCC2)nc1-c1ccc2c(c1)SCC2] | 49 |
| | 5-(2,3-dihydrobenzo[b]thiophen-6-yl)-1-methyl-3- | |
| | (pyrrolidin-1-ylmethyl)-1H-1,2,4-triazole | |
| 58 | 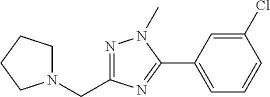 [SMILES: Cn1nc(CN2CCCC2)nc1-c1cccc(Cl)c1] | 23 |
| | 5-(3-chlorophenyl)-1-methyl-3-(pyrrolidin-1-ylmethyl)- | |
| | 1H-1,2,4-triazole | |
| 59 | 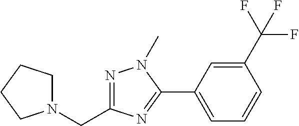 [SMILES: Cn1nc(CN2CCCC2)nc1-c1cccc(C(F)(F)F)c1] | 41 |
| | 1-methyl-3-(pyrrolidin-1-ylmethyl)-5-(3- | |
| | (trifluoromethyl)phenyl)-1H-1,2,4-triazole | |
| 60 | 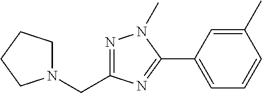 [SMILES: Cc1cccc(-c2nc(CN3CCCC3)nn2C)c1] | 6 |
| | 1-methyl-3-(pyrrolidin-1-ylmethyl)-5-(m-tolyl)-1H-1,2,4- | |
| | triazole | |
| 61 | 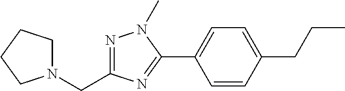 [SMILES: CCCc1ccc(-c2nc(CN3CCCC3)nn2C)cc1] | 8 |
| | 5-(4-propylphenyl)-1-methyl-3-(pyrrolidin-1-ylmethyl)- | |
| | 1H-1,2,4-triazole | |
| 62 | 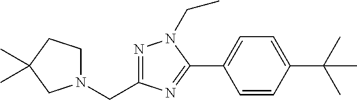 [SMILES: CCn1nc(CN2CCC(C)(C)C2)nc1-c1ccc(C(C)(C)C)cc1] | 101 |
| | 5-(4-(tert-butyl)phenyl)-3-((3,3-dimethylpyrrolidin-1- | |
| | yl)methyl)-1-ethyl-1H-1,2,4-triazole | |
| 63 | 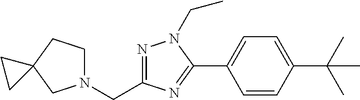 [SMILES: CCn1nc(CN2CCC3(CC3)C2)nc1-c1ccc(C(C)(C)C)cc1] | 102 |
| | 5-((5-(4-(tert-butyl)phenyl)-1-ethyl-1H-1,2,4-triazol-3- | |
| | yl)methyl)-5-azaspiro[2.4]heptane | |
| 64 | 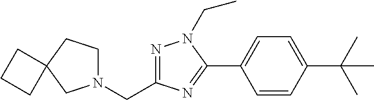 [SMILES: CCn1nc(CN2CCC3(CCC3)C2)nc1-c1ccc(C(C)(C)C)cc1] | 94 |
| | 6-((5-(4-(tert-butylphenyl)-1-ethyl-1H-1,2,4-triazol-3- | |
| | yl)methyl)-6-azaspiro[3.4]octane | |
| 65 | 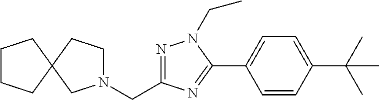 [SMILES: CCn1nc(CN2CCC3(CCCC3)C2)nc1-c1ccc(C(C)(C)C)cc1] | 97 |
| | 2-((5-(4-(tert-butyl)phenyl)-1-ethyl-1H-1,2,4-triazol-3- | |
| | yl)methyl)-2-azaspiro[4.4]nonane | |
| 66 | 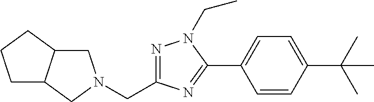 [SMILES: CCn1nc(CN2CC3CCCC3C2)nc1-c1ccc(C(C)(C)C)cc1] | 93 |
| | 2-((5-(4-(tert-butyl)pheny)-1-ethyl-1H-1,2,4-triazol-3- | |
| | yl)methyl)octahydrocyclopenta[c]pyrrole | |
| 67 | 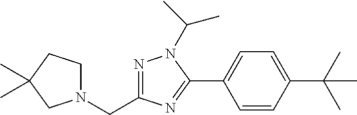 [SMILES: CC(C)n1nc(CN2CCC(C)(C)C2)nc1-c1ccc(C(C)(C)C)cc1] | 97 |
| | 5-(4-(tert-butyl)phenyl)-3-((3,3-dimethylpyrrolidin-1- | |
| | yl)methyl)-1-isopropyl-1H-1,2,4-triazole | |
| 68 | 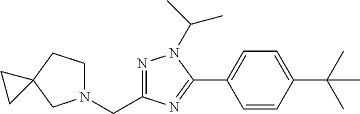 [SMILES: CC(C)n1nc(CN2CCC3(CC3)C2)nc1-c1ccc(C(C)(C)C)cc1] | 93 |
| | 5-((5-(4-(tert-butyl)phenyl)-1-isopropyl-1H-1,2,4-triazol-3- | |
| | yl)methyl)-5-azaspiro[2.4]heptane | |
| 69 | 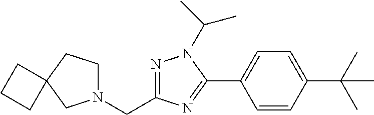 [SMILES: CC(C)n1nc(CN2CCC3(CCC3)C2)nc1-c1ccc(C(C)(C)C)cc1] | 99 |
| | 6-((5-(4-(tert-butyl)phenyl)-1-isopropyl-1H-1,2,4-triazol-3- | |
| | yl)methyl)-6-azaspiro[3.4]octane | |
| 70 | 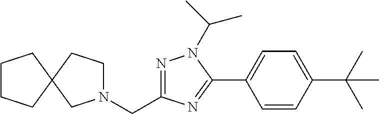 [SMILES: CC(C)n1nc(CN2CCC3(CCCC3)C2)nc1-c1ccc(C(C)(C)C)cc1] | 100 |
| | 2-((5-(4-(tert-butyl)phenyl)-1-isopropyl-1H-1,2,4-triazol-3- | |
| | yl)methyl)-2-azaspiro[4.4]nonane | |
| 71 | 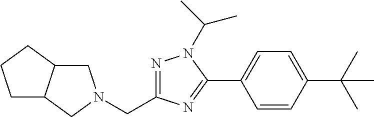 [SMILES: CC(C)n1nc(CN2CC3CCCC3C2)nc1-c1ccc(C(C)(C)C)cc1] | 98 |
| | 2-((5-(4-(tert-butyl)phenyl)-1-isopropyl-1H-1,2,4-triazol-3- | |
| | yl)methyl)octahydrocyclopenta[c]pyrrole | |
| 72 | 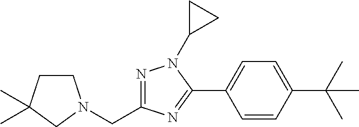 [SMILES: CC1(C)CCN(Cc2nc(-c3ccc(C(C)(C)C)cc3)n(C3CC3)n2)C1] | 95 |
| | 5-(4-(tert-butyl)phenyl)-1-cyclopropyl-3-((3,3- | |
| | dimethylpyrrolidin-1-yl)methyl)-1H-1,2,4-triazole | |
| 73 | 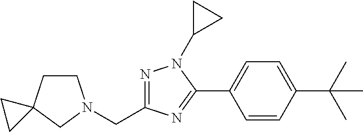 [SMILES: CC(C)(C)c1ccc(-c2nc(CN3CCC4(CC4)C3)nn2C2CC2)cc1] | 100 |
| | 5-((5-(4-(ter-butyl)phenyl)-1-cyclopropyl-1H-1,2,4- | |
| | triazol-3-yl)methyl)-5-azaspiro[2.4]heptane | |
| 74 | 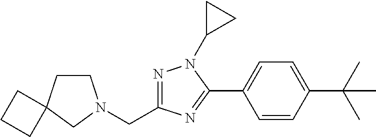 [SMILES: CC(C)(C)c1ccc(-c2nc(CN3CCC4(CCC4)C3)nn2C2CC2)cc1] | 97 |
| | 6-((5-(4-(tert-butyl)phenyl)-1-cyclopropyl-1H-1,2,4- | |
| | triazol-3-yl)methyl)-6-azaspiro[3.4]octane | |
| 75 | 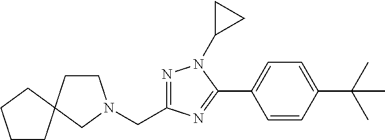 [SMILES: CC(C)(C)c1ccc(-c2nc(CN3CCC4(CCCC4)C3)nn2C2CC2)cc1] | 102 |
| | 2-((5-(4-(tert-butyl)phenyl)-1-cyclopropyl-1H-1,2,4- | |
| | triazol-3-yl)methyl)-2-azaspiro[4.4]nonane | |
| 76 | 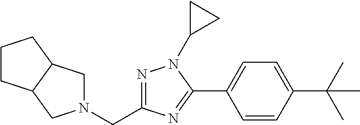 [SMILES: CC(C)(C)c1ccc(-c2nc(CN3CC4CCCC4C3)nn2C2CC2)cc1] | 98 |
| | 2-((5-(4-(tert-butyl)phenyl)-1-cyclopropyl-1H-1,2,4- | |
| | triazol-3-yl)methyl)octahydrocyclopenta[c]pyrrole | |

TABLE 2
Ki Values for the S1R and S2R of Selected Compounds

| Cmpd | S1R Ki (nM) | S2R Ki (nM) |
|---|---|---|
| 1 | 3.72 | 441 |
| 2 | 56 | >10,000 |
| 3 | 131 | >1,000 |
| 4 | 14.3 | >1,000 |
| 5 | 10.2 | 13.9 |
| 6 | 5.52 | 38.5 |
| 7 | 2.02 | 56.6 |
| 8 | 2.28 | 474 |
| 9 | 0.61 | 324 |
| 10 | 9.9 | >1,000 |
| 11 | 22.1 | >1,000 |
| 13 | 20.8 | >1,000 |
| 15 | 35.7 | >1,000 |
| 17 | 32.3 | >1,000 |
| 25 | 37 | >1,000 |
| 26 | 0.89 | 190 |
| 30 | 4.8 | 1710 |
| 31 | 2.16 | 1460 |
| 32 | 13.9 | 750 |
| 65 | 0.52 | 96% at 1 μM |
| 66 | 0.57 | 73% at 1 μM |
| 68 | 0.75 | 360 |
| 75 | 0.56 | 105% at 1 μM |

The term “Ki” or “Ki” refers to the binding constant, which is a particular case of the equilibrium constant, that is associated with the binding and unbinding reaction of the sigma receptor (SR) and either the S1R and S2R ligand (L) molecules, which is formalized as: R + L [symbol] RL

### Example 4: Assessing Whether Compound 8 Causes any Motor Incoordination

Groups of 6 pre-selected male (Sprague-Dawley) rats weighing 180±20 g were included to the study motor coordination. Rats were trained on a rod at a continuous accelerating speed from 4 to 30 rpm/min during a time period of 4 minutes for at least 3 times before the study. Rats were randomly assigned to different groups with similar baseline.

Vehicle and test articles were administered by intraperitoneal (i.p.) injection. The rats were placed on the accelerating rotarod at 30, 60, 90 and 120 min post dosing, and the time (seconds) the rat remained on the rotarod was recorded.

### Example 5: Assessing Dose-Dependent Analgesic Activity of Compound 8 in Rat Formalin Test

One day after the rotarod test, vehicle and test articles were administered intraperitoneally again to groups of 6 Sprague-Dawley male rats weighting 180±20 g, 30 minutes prior to sub-plantar injection of formalin (0.05 ml, 2% solution). The hind paw licking time was recorded during a 0-35 min period at 5-min intervals after formalin challenge as a measure of analgesic activity of the test compound.

### Example 6: Assessing Analgesic Activity of Compound 8 in Paclitaxel-Induced Mechanical Allodynia and Cold Hyperalgesia in Rats

Male Sprague-Dawley rats weighing 240+20 grams were employed. Paclitaxel (brand name TAXOL®, 2 mg/kg) is injected intraperitoneally (i.p.) on days 1, 3, 5 and 7 with a total cumulative dose of 8 mg/kg. During the course of the study, the animals were housed individually under constant temperature, humidity and a 12-hour light-dark cycle.

The behavioral assessments include mechanical allodynia and cold hyperalgesia.

Test articles, vehicle and gabapentin were administered intraperitoneally on Days 14 or 21.

Mechanical allodynia was assessed by the manual von Frey test and measured prior to Paclitaxel injections (Day 0) and test article administration (Days 13 or 20) and at 0.5 hour after dosing of vehicle or test articles on Days 14 or 21. The animals were randomized to each groups based on pre-dose mechanical allodynia values (Days 13 or 20). The animals were given 20 to 30 minutes to acclimatize prior to testing. The paw was touched with a series of 8 manual von Frey monofilaments with logarithmically incremental stiffness [3.61 (0.4 g), 3.84 (0.6 g), 4.08 (1.0 g), 4.31 (2.0 g), 4.56 (4.0 g), 4.74 (6.0 g), 4.93 (8.0 g), and 5.18 (15.0 g)]. The manual von Frey monofilament was applied perpendicularly from underneath the mesh floor to the central plantar surface with sufficient force to cause a slight buckling against the paw, and held for approximately 6-8 seconds. A positive response was noted if the paw is sharply withdrawn; ambulation was considered an ambiguous response, and in such cases, the stimulus was reapplied. Mechanical threshold (50% g threshold) was assessed using the up- and down-method following the procedure described by Chaplan, et al., 1994, J Neurosci Methods 53:55-63.

Cold hyperalgesia was assessed by the manual von Frey test and measured prior to Paclitaxel injections (Day 0) and test article administration (Days 13 or 20) and at 0.5 hour after dosing of vehicle or test articles on Days 14 or 21. The animals were randomized to each groups based on pre-dose cold hyperalgesia values (Days 13 or 20). Test rat was placed in 2.5 cm depth of a 0-2° C. ice water bath in a Plexiglas arena 32×32 cm×27 cm high. Withdrawal latency of the left paw was recorded and averaged twice for cold hyperalgesia assessment. A 20 sec cut time was employed.

All aspects of this work including housing, experimentation, and animal disposal were performed in general accordance with the “Guide for the Care and Use of Laboratory Animals: Eighth Edition” (The National Academies Press, Washington, D C, 2011).

### Example 7: Assessing Analgesic Activity of Compound 8 in STZ-Induced Mechanical Allodynia and Thermal Hyperalgesia in Diabetic Rats

Male Sprague-Dawley rats weighing 225-250 grams were employed. Streptozotocin (STZ, 50 mg/kg) freshly dissolved in 0.9% sterile saline was injected intraperitoneally on day 0. Three days later, diabetes was confirmed by existence of blood glucose >350 mg/dL as measured by glucometer. Animal health was monitored biweekly for 14 days, at which point mechanical allodynia and thermal hyperalgesia were measured and confirmed. During the course of the study, the animals were housed individually under constant temperature, humidity and a 12-hour light-dark cycle. The animals were tested prior to study inclusion for mechanical allodynia and thermal hyperalgesia.

The behavioral assessments include mechanical allodynia and thermal hyperalgesia. Test articles, vehicle and gabapentin were administered intraperitoneally on Days 14 or 21.

Mechanical allodynia was assessed by the manual von Frey test and measured prior to STZ injections (Day 0). The animals were then randomized to each groups based on pre-dose mechanical allodynia values (Day 13 and Day 20). The mechanical allodynia test was performed again at 0.5 and 1.5 hours after vehicle, test article or gabapentin administration on Day 14. The animals were given 20 to 30 minutes to acclimate prior to testing. The paw was touched with a series of 8 manual von Frey monofilaments with logarithmically incremental stiffness [3.61 (0.4 g), 3.84 (0.6 g), 4.08 (1.0 g), 4.31 (2.0 g), 4.56 (4.0 g), 4.74 (6.0 g), 4.93 (8.0 g), and 5.18 (15.0 g)]. The manual von Frey monofilament was applied perpendicularly from underneath the mesh floor to the central plantar surface with sufficient force to cause a slight buckling against the paw, and held for approximately 6-8 seconds. A positive response was noted if the paw was sharply withdrawn; ambulation was considered an ambiguous response, and in such cases, the stimulus was reapplied. Mechanical threshold (50% g threshold) was assessed using the up- and down-method following the procedure described by Chaplan, et al., 1994, J Neurosci Methods 53:55-63).

Thermal hyperalgesia: The animals were tested prior to study inclusion for thermal hyperalgesia on day 13 and Day 20. Thermal hyperalgesia was measured at 1 and 2 hours after dosing of vehicle or test article on Day 14 and Day 21 by using the IITC Model-336G (IITC INC. USA) apparatus. Each rat was placed within a plastic box atop a glass floor for 20 to 30 minutes. A light beam under the floor was aimed at the plantar surface of the left hind paw. The time was measured automatically when the paw was withdrawn away from the thermal stimulus. A cut-off latency of 23 sec was imposed. The latency to withdrawal was obtained for each rat and defined as the heat pain threshold.

### Example 8: Compound 8 Potentiates Morphine's Analgesia in Mouse Models of Acute Pain Studies

Male young CD-1 mice (at the age of 7-8 weeks) were used for this study. Tail flick latency was determined using a lamp device (design derived from D'Amour & Smith, 1941, J. Pharmacol. Exp. Ther. 72(1):74-79). The behavioral endpoint was defined as a flick of the tail out of the path of the light. The intensity of the light was adjusted to provide a baseline response time of approximately 2-4 seconds. The light beam was focused approximately 4 cm from the tip of the tail. A cut off time of 20 seconds was used to prevent tissue damage. Mice received a single intra-peritoneal (IP) injection of either vehicle (saline) or test substance at appropriate doses followed immediately by a single sub-cutaneous (SC) administration of saline or Morphine at the appropriate dose for each group. Thirty minutes after the SC administration, the animals were gently restrained in a manner which allowed access to the tail and positioned beneath a light beam (D'Amour & Smith, 1941, J Pharmacol Exp Ther 72(1):74-79) with the tail aligned for measurement at approximately 4 cm from tail tip. The lamp device automatically timed each tail flick response once the tail was flicked away from the light beam by a sensor beneath the platform. Mice were observed at the time of testing and each animal was tested only once.

Mice were socially housed in solid bottom cages with bedding, which conform to the size recommendations in the most recent Guide for the Care and Use of Laboratory Animals. The animal room was controlled, and controlled with a 12 hour light/dark cycle and were kept clean and vermin-free.

### Example 9: Compound 68 Exhibits Analgesic Activity by Attenuating Mechanical Allodynia and Thermal Hyperalgesia in Spinal Nerve Ligation (SNL) Chung Model in Rats

Groups of 6 male Sprague Dawley rats weighing 160-200 grams were used. Prior to surgery and post-operatively, the animals were housed socially under constant temperature, humidity, and a 12-hour light-dark cycle. Following acclimation to the animal colony for 7 days, the rats were tested for mechanical allodynia and thermal hyperalgesia for baseline (pre-dose) values on Day −1. On Day 0, the animals were anesthetized with pentobarbital (50 mg/kg, i.p.). The left paraspinal muscles were separated from the spinous processes (L4-S2) and the L6-S1 facet joint was nipped. The transverse process of L6 was removed to allow for identifying the location of the L5 and L6 spinal nerves. The left L5-L6 spinal nerves were isolated and tightly ligated with 6.0 silk sutures. The rats are allowed to recover for 13 days.

Rats were tested for mechanical allodynia and thermal hyperalgesia following acclimation on Day −1 for baseline (pre-dose) values. Following the recovery period, the rats were tested to verify the mechanical allodynia on Day 13. The rats were pre-selected for experimentation only if the pain threshold after nerve ligation (pre-treatment) is reduced by 10 grams of force relative to the response of the individual paw before nerve ligation (pre-ligation), namely, with clear presence of allodynia. The rats were randomized based on pre-dose mechanical allodynia scores to balanced treatment groups.

The manual von Frey test was repeated again 15 and 75 minutes following administration of the vehicle, compound 68 at two doses, or the positive control, gabapentin 50 mg/kg, IP, on Day 14 post-surgery.

Following a four (4) day wash out “period”, the rats were tested in the thermal hyperalgesia test and randomized to balance treatment groups. On Day 19, the rats were administered vehicle, Compound 68 at two doses, or gabapentin 50 mg/kg, IP. At 15 and 75 minutes post-dose the thermal hyperalgesia is tested again.

Mechanical allodynia: The mechanical allodynia is assessed by the manual von Frey test (Chaplan up/down method using von Frey filaments on the plantar surface of the CFA injected paw). The animals are allowed 20 to 30 minutes to acclimatize to individual compartments on a wire mesh rack prior to testing. The right paw is touched with a series of 8 manual von Frey monofilaments with logarithmically incremental stiffness [3.61 (0.4 g), 3.84 (0.6 g), 4.08 (1.0 g), 4.31 (2.0 g), 4.56 (4.0 g), 4.74 (6.0 g), 4.93 (8.0 g), and 5.18 (15.0 g)]. The manual von Frey monofilament is applied perpendicularly from underneath the mesh floor to the central plantar surface with sufficient force to cause a slight buckling against the paw, and held for approximately 6-8 seconds. A positive response is noted if the paw is sharply withdrawn; ambulation is considered an ambiguous response, and in such cases, the stimulus is reapplied. Mechanical threshold (50% g threshold) is assessed using the up- and down-method following the procedure described by Chaplan (Chaplan et al., 1994, J Neurosci Methods 53:55-63).

Thermal hyperalgesia: Thermal hyperalgesia is measured by the Hargreaves method. Each rat is placed within a plastic box atop a glass floor for at least 20 minutes to acclimate to the apparatus to allow the testing time of 15 and 75 minutes post-dosing. A light beam under the floor is aimed at the plantar surface of one hind paw. The time is measured automatically when the paw is withdrawn away from the thermal stimulus. A cut-off latency of 23 sec is imposed. The latency to withdrawal is obtained for each rat and defined as the heat pain threshold.

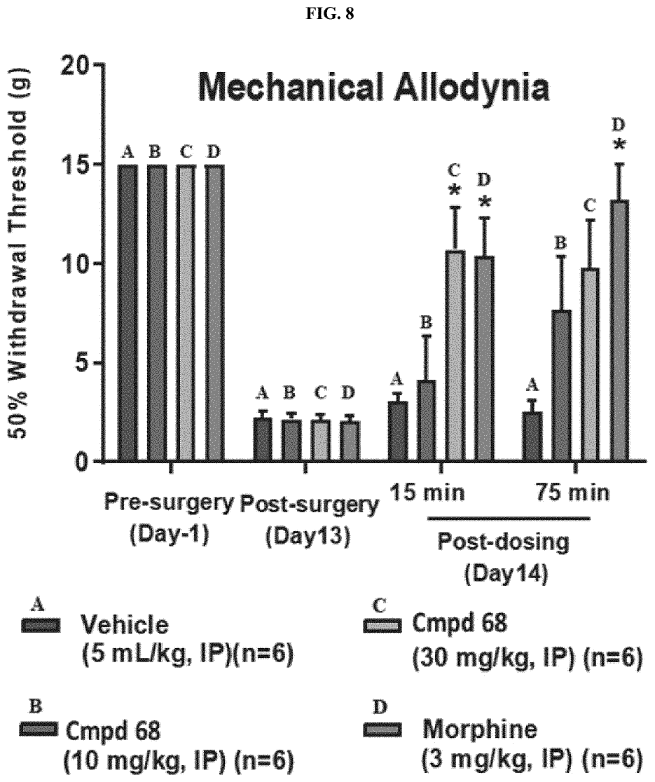

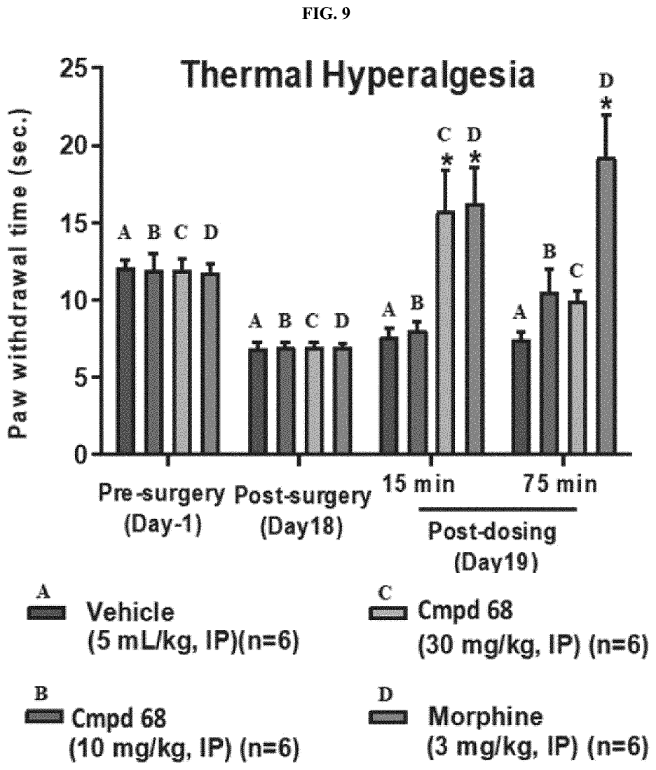

Experimental results indicate that compound 68 exhibits analgesic activity by attenuating the mechanical allodynia (FIG. 8) and thermal hyperalgesia (FIG. 9) in spinal nerve ligand (SNL) Chung model in rats.

### Example 10: Chemical Characterization

Selected compounds of the invention were prepared using methods illustrated elsewhere herein, and the ¹H-NMR data for those compounds is illustrated herein:

Compound 1: ¹H-NMR (400 MHz, CDCl₃) δ 7.63 (d, J=7.2 Hz, 2H), 7.53 (d, J=7.7 Hz, 2H), 3.98 (s, 3H), 3.63 (s, 3.73 (m, 2H), 2.62 (b, 4H), 1.67 (b, 4H), 1.37 (b, 2H), 1.27 (s, 9H).

Compound 2: ¹H-NMR (400 MHz, CDCl₃) δ 7.63 (d, J=7.2 Hz, 2H), 7.53 (d, J=7.1 Hz, 2H), 3.98 (s, 3H), 3.63 (s, 3.73 (b, 2H), 3.72 (b, 2H), 2.67 (b, 4H), 1.37 (s, 9H).

Compound 3: ¹HNMR (400 MHz, CDCl₃) δ 7.62 (d, J=7.2 Hz, 2H), 7.51 (d, J=7.1 Hz, 2H), 3.97 (s, 3H), 3.63 (s, 3.73 (s, 2H), 2.58 (b, 8H), 2.34 (s, 3H), 1.37 (s, 9H).

Compound 4: ¹H-NMR (400 MHz, CDCl₃) ¹HNMR (400 MHz, CDCl₃) δ 7.62 (d, J=7.2 Hz, 2H), 7.51 (d, J=7.1 Hz, 2H), 7.25 (m, 4H), 3.98 (s, 3H), 3.73 (b, 2H), 3.27 (b, 4H), 2.81 (b, 4H), 1.26 (s, 9H).

Compound 5: ¹H-NMR (400 MHz, CDCl₃) δ 7.59 (d, J=7.2 Hz, 2H), 7.50 (d, J=7.1 Hz, 2H), 7.29 (m, 2H), 7.20 (s, 3H), 4.13 (s, 3H), 3.93 (b, 2H), 3.12 (b, 2H), 2.66 (B, 10H), 1.27 (s, 9H).

Compound 6: ¹H-NMR (400 MHz, CDCl₃) δ 7.63 (d, J=7.2 Hz, 2H), 7.53 (d, J=7.7 Hz, 2H), 7.32 (m, 4H), 7.26 (s, 1H), 3.98 (s, 3H), 3.71 (s, 2H), 3.56 (m, 2H), 2.66 (b, 8H), 1.37 (s, 9H).

Compound 7: ¹H-NMR (400 MHz, CDCl₃) δ 7.63 (d, J=7.2 Hz, 2H), 7.60 (d, J=7.2 Hz, 2H), 7.52 (m, 3H), 7.14 (m, 2H), 3.97 (s, 3H), 3.66 (s, 2H), 3.05 (m, 2H), 2.55 (d, J=3.5 Hz, 2H), 2.34 (m, 2H), 1.68 (m, 2H), 1.59 (b, 1H), 1.39 (m, 2H), 1.27 (s, 9H).

Compound 8: ¹H-NMR (400 MHz, CDCl₃) δ 7.61 (d, J=7.2 Hz, 2H), 7.49 (d, J=7.1 Hz, 2H), 3.96 (s, 3H), 3.78 (s, 2H), 2.70 (b, 4H), 1.82 (b, 4H), 1.36 (s, 9H).

Compound 9: ¹H-NMR (400 MHz, CDCl₃) δ 7.63 (d, J=7.2 Hz, 2H), 7.52 (d, J=7.1 Hz, 2H), 3.97 (s, 3H), 3.82 (b, 2H), 2.49 (b, 5H), 1.37 (s, 9H), 1.04 (b, 1H), 0.55 (b, 2H), 0.19 (b, 2H).

Compound 10: 7.68-7.63 (m, 4H), 4.36 (s, 2H), 4.03 (s, 3H), 3.76 (d, J=8 Hz, 2H), 3.31 (t, J=8 Hz, 2H), 2.81 (t, J=8 Hz, 2H), 1.91 (s, 2H), 1.44 (t, J=8 Hz, 2H), 1.09 (s, 2H).

Compound 11: 7.82 (d, J=8 Hz, 1H), 7.64 (d, J=8 Hz, 1H), 7.55-7.52 (m, 1H), 4.35 (s, 2H), 4.04 (s, 3H), 3.79 (t, J=4 Hz, 2H), 3.28-3.26 (m, 2H), 2.21 (d, J=4 Hz, 2H), 1.93 (s, 2H).

Compound 12: 7.83 (s, 4H), 4.37 (s, 2H), 4.06 (s, 3H), 3.79 (t, J=8 Hz, 2H), 3.30-3.28 (m, 2H), 2.22 (t, J=4 Hz, 2H), 1.94 (t, J=8 Hz, 2H).

Compound 13: 7.79 (d, J=8 Hz, 1H), 7.64 (s, 1H), 7.58 (t, J=8 Hz, 1H), 4.36 (s, 2H), 4.04 (s, 3H), 3.30-3.27 (m, 2H), 2.21-2.19 (m, 2H), 2.59 (s, 3H), 2.21-2.19 (m, 2H), 1.95-1.91 (m, 2H).

Compound 14: 7.63 (d, J=8 Hz, 2H), 7.42 (d, J=8 Hz, 2H), 4.42 (s, 2H), 4.05 (s, 3H), 3.77 (s, 2H), 3.35 (s, 2H), 3.04-2.97 (m, 1H), 2.19 (s, 2H), 1.95 (s, 2H), 1.30 (d, J=8 Hz, 6H).

Compound 15: 7.37 (t, J=8 Hz, 2H), 7.24 (t, J=8 Hz, 1H), 4.37 (s, 2H), 4.01 (s, 3H), 3.75 (t, J=4 Hz, 2H), 3.34 (d, J=4 Hz, 2H), 2.85 (s, 4H), 2.18 (d, J=4 Hz, 2H), 1.92 (s, 2H), 1.85 (s, 4H).

Compound 16: 7.66 (s, 1H), 7.59 (d, J=8 Hz, 1H), 7.55-7.27 (m, 2H), 4.37 (s, 2H), 4.01 (s, 3H), 3.78-3.74 (m, 2H), 3.34 (t, J=8 Hz, 2H), 2.19 (t, J=8 Hz, 2H), 1.93 (t, J=8 Hz, 2H), 1.38 (s, 2H).

Compound 17: 7.76 (s, 1H), 7.64 (t, J=8 Hz, 2H), 7.54 (t, J=8 Hz, 1H), 4.36 (s, 2H), 4.02 (s, 3H), 3.76 (s, 2H), 3.31 (t, J=4 Hz, 2H), 2.20 (d, J=4 Hz, 2H), 1.93 (s, 2H), 1.46 (t, J=4 Hz, 2H), 1.11 (s, 2H).

Compound 18: 7.64 (d, J=8 Hz, 2H), 7.54 (d, J=8 Hz, 2H), 4.36 (s, 2H), 4.02 (s, 3H), 3.79-3.75 (m, 2H), 3.31-3.27 (m, 2H), 2.21-2.18 (m, 2H), 1.94-1.90 (m, 2H).

Compound 19: 7.49 (s, 1H), 7.41 (d, J=9.2 Hz, 1H), 7.32 (d, J=9.2 Hz, 1H), 4.43 (s, 2H), 4.04 (s, 3H), 3.78-3.82 (m, 2H), 3.33-3.36 (m, 2H), 2.36 (s, 6H), 1.99 (s, 2H), 1.95-1.98 (m, 3H).

Compound 20: 7.57 (d, J=8.4 Hz, 2H), 7.35 (d, J=7.6 Hz, 2H), 4.36 (s, 2H), 4.01 (s, 3H), 3.74-3.76 (m, 2H), 3.31-3.35 (m, 2H), 2.45 (s, 3H), 2.16-2.20 (m, 2H), 1.91 (s, 2H).

Compound 21: 7.65 (d, J=8 Hz, 2H), 7.25 (s, 2H) 4.52 (s, 2H), 4.09 (s, 3H), 3.79 (s, 2H), 3.36 (s, 2H), 2.21-2.22 (d, J=8.4 Hz, 2H), 1.98-2.02 (m, 3H), 1.10-1.13 (m, 2H), 0.80-0.81 (m, 2H) Compound 22: 7.56 (s, 2H), 7.39-7.46 (m, 2H), 4.44 (s, 2H), 4.05 (s, 3H), 3.76-3.78 (d, J=5.2 Hz, 2H) 3.37-3.38 (d, J=5.2 Hz, 2H), 2.99-3.02 (m, 4H), 2.12-2.20 (m, 4H), 1.97 (S, 2H).

Compound 23: 8.22 (s, 1H), 7.97-7.99 (d, J=8 Hz, 1H), 7.66 (t, J=4 Hz, 1H), 7.44-7.45 (d, J=5.6 Hz, 1H), 4.39 (s, 2H), 4.08 (s, 3H), 3.77 (s, 2H), 3.34 (s, 2H), 2.18-2.21 (d, J=4.8 Hz, 2H), 1.93 (s, 2H).

Compound 24: 7.84 (s, 1H), 7.64-7.65 (d, J=2.4 Hz, 1H), 7.55-7.57 (d, J=8 Hz, 2H), 7.49-7.51 (d, J=8 Hz, 1H), 7.32-7.34 (m, 1H), 4.35-4.37 (d, J=9.2 Hz, 2H), 4.04-4.08 (d, J=16 Hz, 3H), 3.78 (s, 2H), 3.29 (s, 2H), 2.21 (s, 2H), 1.94-1.95 (d, J=5.6 Hz, 2H).

Compound 25: (400 MHz, DMSO-d₆): δ 10.97 (br s, 1H), 8.47 (s, 1H), 7.98 (d, J=8.44 Hz, 1H), 7.84 (d, J=8.31 Hz, 1H), 7.67 (s, 1H), 4.49 (br d, J=5.01 Hz, 2H), 4.05 (s, 3H), 3.57 (br d, J=5.01 Hz, 2H), 3.22 (br d, J=7.21 Hz, 2H), 1.86-2.03 (m, 4H)

Compound 26: (400 MHz, DMSO-d₆): δ 11.36 (br s, 1H), 7.74 (d, J=8.6 Hz, 2H), 7.60 (d, J=8.4 Hz, 2H), 4.37-4.52 (m, 2H), 4.00 (s, 3H), 3.65 (qd, J=6.0, 12.0 Hz, 1H), 3.29-3.46 (m, 2H), 3.04 (dd, J=7.5, 11.2 Hz, 1H), 1.70-1.89 (m, 2H), 1.33 (s, 9H), 1.10 (d, J=11.9 Hz, 6H)

Compound 27: (400 MHz, DMSO-d₆): δ 11.19 (br s, 1H), 7.74 (d, J=8.4 Hz, 2H), 7.60 (d, J=8.4 Hz, 2H), 4.42-4.52 (m, 2H), 4.00 (s, 3H), 3.61 (qd, J=6.0, 11.9 Hz, 1H), 3.31-3.50 (m, 2H), 3.16 (dd, J=7.1, 11.5 Hz, 1H), 1.75-1.97 (m, 2H), 1.71-1.49 (m, 8H), 1.33 (s, 9H)

Compound 28: (400 MHz, DMSO-d₆): δ 11.27 (br s, 1H), 7.74 (d, J=8.6 Hz, 2H), 7.60 (d, J=8.4 Hz, 2H), 4.45-4.53 (m, 1H), 4.53 (br s, 1H), 4.00 (s, 3H), 3.65-3.76 (m, 1H), 3.41-3.51 (m, 1H), 3.24-3.38 (m, 2H), 1.83-2.03 (m, 2H), 1.33 (s, 9H), 0.55-0.72 (m, 4H)

Compound 29: (400 MHz, DMSO-d₆): δ 11.02 (br s, 1H), 7.74 (d, J=8.6 Hz, 2H), 7.60 (d, J=8.6 Hz, 2H), 4.43 (br d, J=4.2 Hz, 2H), 4.00 (s, 3H), 3.51-3.60 (m, 2H), 3.15-3.38 (m, 2H), 2.06-2.18 (m, 2H), 1.69-2.04 (m, 6H), 1.33 (s, 9H)

Compound 30: (400 MHz, DMSO-d₆): δ 10.97 (br s, 1H), 7.56-7.70 (m, 4H), 4.48 (d, J=5.01 Hz, 2H), 4.27 (q, J=7.21 Hz, 2H), 3.45-3.64 (m, 2H), 3.16-3.26 (m, 2H), 1.81-2.05 (m, 4H), 1.41 (t, J=7.15 Hz, 3H), 1.33 (s, 9H)

Compound 31: (400 MHz, DMSO-d₆): δ 10.99-11.19 (m, 1H), 7.57-7.63 (m, 4H), 4.70 (quin, J=6.56 Hz, 1H), 4.48 (d, J=5.07 Hz, 2H), 3.55 (br dd, J=10.25, 4.96 Hz, 2H), 3.14-3.27 (m, 2H), 1.83-2.03 (m, 4H), 1.44 (d, J=6.39 Hz, 6H), 1.33 (s, 9H)

Compound 32: (400 MHz, DMSO-d₆): δ 11.27 (br s, 1H), 7.91 (d, J=8.4 Hz, 2H), 7.60 (d, J=8.6 Hz, 2H), 4.42 (d, J=4.9 Hz, 2H), 3.90-3.97 (m, 1H), 3.54 (br dd, J=5.0, 10.3 Hz, 2H), 3.14-3.22 (m, 2H), 1.85-2.00 (m, 4H), 1.31-1.35 (m, 9H), 1.03-1.14 (m, 4H)

Compound 33: (400 MHz, DMSO-d₆): δ 10.37-11.12 (m, 1H), 7.89-7.97 (m, 2H), 7.47-7.54 (m, 2H), 4.74 (br s, 2H), 4.29 (q, J=7.21 Hz, 2H), 3.65 (br d, J=3.30 Hz, 2H), 3.15-3.28 (m, 2H), 1.89-2.12 (m, 4H), 1.43 (t, J=7.21 Hz, 3H), 1.31 (s, 9H)

Compound 34: (400 MHz, DMSO-d₆): δ 11.39-11.60 (m, 1H), 7.93 (d, J=8.31 Hz, 2H), 7.49 (d, J=8.44 Hz, 2H), 4.89 (quin, J=6.39 Hz, 1H), 4.75 (br d, J=3.06 Hz, 2H), 3.63 (br s, 2H), 3.27 (br s, 2H), 2.06 (br s, 2H), 1.93 (br d, J=4.65 Hz, 2H), 1.47 (d, J=6.48 Hz, 6H), 1.31 (s, 9H)

Compound 35: ¹H-NMR (400 MHz, CDCl₃) δ 7.17 (d, J=7.3 Hz, 2H), 7.02 (d, J=6.9 Hz, 2H), 4.46 (s, 2H), 3.97 (s, 3H), 2.39 (m, 4H), 1.62 (m, 4H), 1.27 (s, 9H).

Compound 36: ¹H-NMR (400 MHz, CDCl₃) δ 7.29 (d, J=7.2 Hz, 2H), 7.21 (d, J=7.0 Hz, 2H), 4.41 (s, 2H), 2.31 (s, 3H), 2.25 (m, 4H), 1.51 (m, 4H), 0.41 (b, 4H).

Compound 37: ¹H-NMR (400 MHz, CDCl₃) δ 7.21 (m, 2H), 7.14 (d, J=7.0 Hz, 1H), 4.46 (s, 2H), 2.36 (s, 3H), 2.27 (m, 4H), 1.56 (m, 4H).

Compound 38: ¹H-NMR (400 MHz, CDCl₃) δ 7.14 (d, J=7.0 Hz, 1H), 7.19 (m, 2H), 4.41 (s, 2H), 2.32 (s, 3H), 2.27 (m, 4H), 1.56 (m, 4H).

Compound 39: (400 MHz, DMSO-d₆): δ 10.98-11.08 (m, 1H), 7.88-7.94 (m, 4H), 7.77 (d, J=7.3 Hz, 2H), 7.52 (t, J=7.6 Hz, 2H), 7.41-7.46 (m, 1H), 4.49 (br d, J=5.1 Hz, 2H), 4.05 (s, 3H), 3.54-3.61 (m, 2H), 3.18-3.26 (m, 2H), 1.88-2.04 (m, 4H)

Compound 40: (400 MHz, DMSO-d₆): δ 11.04 (br s, 1H), 8.04 (s, 1H), 7.88 (d, J=7.83 Hz, 1H), 7.66-7.82 (m, 4H), 7.51 (t, J=7.16 Hz, 2H), 7.40-7.46 (m, 1H), 4.42-4.58 (m, 2H), 4.06 (s, 3H), 3.51-3.62 (m, 2H), 3.16-3.27 (m, 2H), 2.00 (br s, 2H), 1.84-1.97 (m, 2H)

Compound 41: (400 MHz, DMSO-d₆): δ 11.24 (br s, 1H), 7.82 (d, J=8.8 Hz, 2H), 7.46 (t, J=7.8 Hz, 2H), 7.20-7.26 (m, 1H), 7.09-7.17 (m, 4H), 4.44 (br d, J=4.9 Hz, 2H), 3.94-4.01 (m, 3H), 3.54 (br d, J=5.3 Hz, 2H), 3.19 (br dd, J=6.7, 10.5 Hz, 2H), 1.85-2.02 (m, 4H)

Compound 42: (400 MHz, DMSO-d₆): δ 11.11 (br s, 1H), 7.51-7.66 (m, 2H), 7.39-7.46 (m, 3H), 7.15-7.26 (m, 2H), 7.09 (d, J=7.83 Hz, 2H), 4.41-4.48 (m, 2H), 3.99 (s, 3H), 3.52 (br dd, J=4.89, 10.27 Hz, 2H), 3.11-3.23 (m, 2H), 1.82-2.02 (m, 4H)

Compound 43: (400 MHz, DMSO-d₆): δ 11.36 (br s, 1H), 7.75 (d, J=8.6 Hz, 2H), 7.46-7.50 (m, 2H), 7.40 (t, J=7.3 Hz, 2H), 7.31-7.36 (m, 1H), 7.20 (d, J=8.6 Hz, 2H), 5.20 (s, 2H), 4.43 (br d, J=4.2 Hz, 2H), 3.99-3.94 (m, 3H), 3.54 (br d, J=5.1 Hz, 2H), 3.15-3.25 (m, 2H), 1.85-2.00 (m, 4H)

Compound 44: (400 MHz, DMSO-d₆): δ 11.18 (br s, 1H), 7.32-7.53 (m, 8H), 7.24 (d, J=7.54 Hz, 1H), 5.20 (s, 2H), 4.45 (d, J=4.77 Hz, 2H), 3.96 (s, 3H), 3.55 (br d, J=5.14 Hz, 2H), 3.19 (br dd, J=6.91, 10.58 Hz, 2H), 1.83-2.03 (m, 4H)

Compound 45: (400 MHz, DMSO-d₆): δ 11.41 (br s, 1H), 7.72 (d, J=8.2 Hz, 2H), 7.41 (d, J=8.2 Hz, 2H), 4.43 (br d, J=4.4 Hz, 2H), 3.94-4.01 (m, 3H), 3.54 (br d, J=5.1 Hz, 2H), 3.15-3.24 (m, 2H), 2.54-2.62 (m, 1H), 1.85-2.01 (m, 4H), 1.80 (br d, J=7.7 Hz, 4H), 1.70 (br d, J=12.3 Hz, 1H), 1.32-1.49 (m, 4H), 1.19-1.29 (m, 1H)

Compound 46: (400 MHz, DMSO-d₆): δ 11.13 (br s, 1H), 7.74 (d, J=8.4 Hz, 2H), 7.59 (d, J=8.4 Hz, 2H), 4.33-4.42 (m, 2H), 3.99 (s, 3H), 3.54-3.62 (m, 2H), 3.47 (br d, J=9.5 Hz, 2H), 1.72 (br s, 2H), 1.33 (s, 9H), 1.04 (br d, J=4.4 Hz, 1H), 0.58-0.68 (m, 1H)

Compound 47: (400 MHz, DMSO-d₆): δ 10.92-11.23 (m, 1H), 7.74 (d, J=8.2 Hz, 2H), 7.60 (d, J=8.4 Hz, 2H), 4.47 (br d, J=4.6 Hz, 1H), 4.39 (br d, J=3.7 Hz, 1H), 3.99 (s, 3H), 3.77 (br s, 1H), 3.36-3.46 (m, 1H), 3.28 (br d, J=11.2 Hz, 1H), 2.68-2.86 (m, 3H), 1.44-1.81 (m, 6H), 1.33 (s, 9H)

Compound 48: (400 MHz, DMSO-d₆): δ 10.67 (br s, 1H), 7.75 (d, J=8.2 Hz, 2H), 7.60 (d, J=8.2 Hz, 2H), 4.33 (br d, J=4.2 Hz, 2H), 4.26 (br s, 2H), 4.00 (s, 3H), 2.21 (br d, J=8.8 Hz, 2H), 2.02 (br d, J=7.8 Hz, 2H), 1.77 (br d, J=8.4 Hz, 2H), 1.68 (br d, J=7.7 Hz, 2H), 1.33 (s, 9H)

Compound 49: ¹H-NMR (400 MHz, CDCl₃) δ 8.03 (s, 1H), 7.82 (m, 1H), 7.35 (m, 3H), 4.45 (s, 2H), 2.37 (s, 3H), 2.23 (m, 4H), 1.55 (m, 4H).

Compound 50: ¹H-NMR (400 MHz, CDCl₃) δ 7.09 (m, 2H), 6.89 (d, J=6.2 Hz, 1H), 4.39 (s, 2H), 2.69 (m, 4H), 2.30 (s, 3H), 2.26 (m, 4H), 1.76 (m, 4H), 1.49 (s, 4H).

Compound 51: ¹H-NMR (400 MHz, CDCl₃) δ 7.26 (m, 4H), 4.29 (s, 2H), 2.39 (m, 1H), 2.31 (s, 3H), 2.25 (m, 4H), 1.60 (m, 6H), 1.09 (m, 8H).

Compound 52: ¹H-NMR (400 MHz, CDCl₃) δ 7.79 (d, J=7.6 Hz, 2H), 7.52 (m, 5H), 7.29 (d, J=7.3 Hz, 2H), 4.46 (s, 2H), 2.34 (s, 3H), 2.25 (m, 4H), 1.51 (m, 4H).

Compound 53: ¹H-NMR (400 MHz, CDCl₃) δ 8.49 (d, J=7.4 Hz, 2H), 7.37 (d, J=7.2 Hz, 2H), 3.65 (s, 2H), 3.62 (s, 3H), 2.54 (m, 4H), 1.45 (m, 4H), 1.31 (s, 9H).

Compound 54: ¹H-NMR (400 MHz, CDCl₃) δ 8.39 (d, J=7.2 Hz, 2H), 7.31 (d, J=7.0 Hz, 2H), 3.63 (s, 2H), 2.62 (m, 1H), 2.54 (m, 4H), 1.59 (m, 4H), 1.31 (s, 9H), 0.54 (b, 4H).

Compound 62: (400 MHz, DMSO-d₆) δ 11.42 (br s, 1H), 7.71-7.56 (m, 4H), 4.46 (br d, J=3.9 Hz, 2H), 4.28 (q, J=7.2 Hz, 2H), 3.65 (qd, J=5.9, 11.9 Hz, 1H), 3.48-3.30 (m, 2H), 3.05 (br dd, J=7.3, 11.4 Hz, 1H), 1.86-1.68 (m, 2H), 1.41 (t, J=7.2 Hz, 3H), 1.33 (s, 9H), 1.10 (d, J=19.2 Hz, 5H).

Compound 63: (400 MHz, DMSO-d₆) δ 11.21 (br s, 1H), 7.71-7.54 (m, 4H), 4.52 (br s, 2H), 4.28 (q, J=7.2 Hz, 2H), 3.78-3.64 (m, 1H), 3.52-3.41 (m, 1H), 3.38-3.22 (m, 2H), 2.03-1.83 (m, 2H), 1.41 (t, J=7.2 Hz, 3H), 1.33 (s, 9H), 0.73-0.53 (m, 4H).

Compound 64: (400 MHz, DMSO-d₆) δ 10.97 (br s, 1H), 7.68-7.59 (m, 4H), 4.45 (d, J=4.8 Hz, 2H), 4.48-4.40 (m, 2H), 4.28 (q, J=7.2 Hz, 2H), 3.61-3.52 (m, 2H), 3.36-3.27 (m, 1H), 3.23 (dd, J=6.4, 11.8 Hz, 1H), 2.17-2.07 (m, 2H), 2.02-1.73 (m, 6H), 1.42 (t, J=7.2 Hz, 3H), 1.34 (s, 9H).

Compound 65: (400 MHz, DMSO-d₆) δ 11.15 (br s, 1H), 7.68-7.59 (m, 4H), 4.48 (br d, J=4.8 Hz, 2H), 4.28 (q, J=7.2 Hz, 2H), 3.67-3.57 (m, 1H), 3.49-3.34 (m, 2H), 3.18 (dd, J=7.0, 11.6 Hz, 1H), 1.96-1.75 (m, 2H), 1.70-1.51 (m, 8H), 1.41 (t, J=7.2 Hz, 3H), 1.34 (s, 9H).

Compound 66: (400 MHz, DMSO-d₆) δ 11.00-10.76 (m, 1H), 7.68-7.59 (m, 4H), 4.51-4.39 (m, 2H), 4.27 (q, J=7.3 Hz, 2H), 3.78 (br s, 1H), 3.48-3.23 (m, 1H), 2.93-2.63 (m, 4H), 1.81-1.62 (m, 2H), 1.62-1.47 (m, 4H), 1.41 (t, J=7.2 Hz, 3H), 1.31-1.29 (m, 9H).

Compound 67: (400 MHz, DMSO-d₆) δ 11.21 (br s, 1H), 7.66-7.55 (m, 4H), 4.71 (td, J=6.5, 13.0 Hz, 1H), 4.47 (br d, J=4.5 Hz, 2H), 3.65 (qd, J=5.9, 11.9 Hz, 1H), 3.46-3.31 (m, 2H), 3.04 (br dd, J=7.4, 11.4 Hz, 1H), 1.84-1.71 (m, 2H), 1.44 (d, J=6.5 Hz, 6H), 1.33 (s, 9H), 1.09 (d, J=18.6 Hz, 6H).

Compound 68: (400 MHz, DMSO-d₆) δ 11.12 (br s, 1H), 7.65-7.55 (m, 4H), 4.71 (td, J=6.5, 13.1 Hz, 1H), 4.53 (br d, J=3.3 Hz, 2H), 3.71 (br d, J=4.8 Hz, 1H), 3.53-3.42 (m, 1H), 3.40-3.22 (m, 2H), 2.06-1.82 (m, 2H), 1.44 (d, J=6.5 Hz, 6H), 1.33 (s, 9H), 0.75-0.55 (m, 4H).

Compound 69: (400 MHz, DMSO-d₆) δ 11.33 (br s, 1H), 7.67-7.52 (m, 4H), 4.71 (spt, J=6.5 Hz, 1H), 4.50-4.34 (m, 2H), 3.61-3.47 (m, 2H), 3.37-3.16 (m, 2H), 2.19-2.02 (m, 2H), 2.02-1.67 (m, 6H), 1.44 (d, J=6.6 Hz, 6H), 1.33 (s, 9H).

Compound 70: (400 MHz, DMSO-d₆) δ 11.54 (br s, 1H), 7.66-7.52 (m, 4H), 4.71 (spt, J=6.5 Hz, 1H), 4.44 (d, J=4.4 Hz, 2H), 3.61 (qd, J=6.0, 11.8 Hz, 1H), 3.49-3.31 (m, 2H), 3.18 (dd, J=6.7, 11.6 Hz, 1H), 1.90-1.75 (m, 2H), 1.70-1.46 (m, 8H), 1.44 (d, J=6.6 Hz, 6H), 1.33 (s, 9H).

Compound 71: (400 MHz, DMSO-d₆) δ 11.01-10.67 (m, 1H), 7.67-7.53 (m, 4H), 4.70 (br d, J=6.5 Hz, 1H), 4.52-4.44 (m, 2H), 3.78 (br s, 1H), 3.47-3.24 (m, 1H), 2.94-2.55 (m, 4H), 1.83-1.47 (m, 6H), 1.44 (d, J=6.5 Hz, 6H), 1.33 (s, 9H).

Compound 72: (400 MHz, DMSO-d₆) δ 11.17 (br s, 1H), 7.91 (d, J=8.4 Hz, 2H), 7.61 (d, J=8.4 Hz, 2H), 4.43 (br d, J=2.4 Hz, 2H), 3.94 (tt, J=3.7, 7.2 Hz, 1H), 3.64 (qd, J=6.1, 12.0 Hz, 1H), 3.44-3.26 (m, 2H), 3.02 (br dd, J=7.0, 11.3 Hz, 1H), 1.87-1.70 (m, 2H), 1.34 (s, 9H), 1.18-1.02 (m, 10H).

Compound 73: (400 MHz, DMSO-d₆) δ 11.20 (br s, 1H), 7.91 (d, J=8.4 Hz, 2H), 7.61 (d, J=8.4 Hz, 2H), 4.48 (br s, 2H), 3.94 (tt, J=3.7, 7.2 Hz, 1H), 3.70 (br d, J=4.2 Hz, 1H), 3.51-3.39 (m, 1H), 3.37-3.22 (m, 2H), 2.04-1.83 (m, 2H), 1.33 (s, 9H), 1.17-0.99 (m, 4H), 0.74-0.51 (m, 4H).

Compound 74: (400 MHz, DMSO-d₆) δ 11.13 (br s, 1H), 7.94-7.88 (m, 2H), 7.63-7.57 (m, 2H), 4.39 (d, J=4.9 Hz, 2H), 3.94 (tt, J=3.7, 7.2 Hz, 1H), 3.59-3.48 (m, 2H), 3.34-3.16 (m, 2H), 2.15-2.05 (m, 2H), 2.01-1.71 (m, 6H), 1.33 (s, 9H), 1.16-1.01 (m, 4H).

Compound 75: (400 MHz, DMSO-d₆) δ 11.59 (br s, 1H), 7.91 (d, J=8.6 Hz, 2H), 7.60 (d, J=8.4 Hz, 2H), 4.40 (d, J=4.4 Hz, 2H), 3.99-3.88 (m, 1H), 3.60 (qd, J=6.0, 11.9 Hz, 1H), 3.48-3.30 (m, 2H), 3.16 (dd, J=7.1, 11.5 Hz, 1H), 1.92-1.77 (m, 2H), 1.73-1.47 (m, 8H), 1.33 (s, 9H), 1.18-1.02 (m, 4H).

Compound 76: (400 MHz, DMSO-d₆) δ 10.74-10.44 (m, 1H), 7.92 (d, J=8.4 Hz, 2H), 7.61 (d, J=8.6 Hz, 2H), 4.47 (br d, J=5.1 Hz, 2H), 3.94 (td, J=3.4, 7.2 Hz, 1H), 3.77 (br s, 1H), 3.45-3.25 (m, 1H), 2.74 (br s, 4H), 1.82-1.62 (m, 2H), 1.62-1.45 (m, 4H), 1.34 (s, 9H), 1.17-1.10 (m, 2H), 1.10-1.02 (m, 2H).

### ENUMERATED EMBODIMENTS

The following exemplary embodiments are provided, the numbering of which is not to be construed as designating levels of importance.

Embodiment 1 provides a compound of formula (I), or a salt, solvate, isotopically labeled, enantiomer, diastereoisomer, or tautomer thereof:

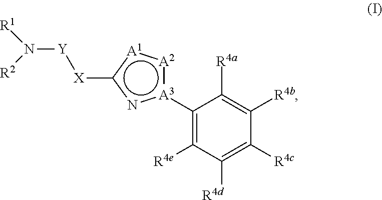

wherein:

A¹, A², and A³ are such that one applies: (a) A¹=N, A²=NR³, and A³=C; (b) A¹=NR³, A²=N, and A³=C; or (c) A¹=N, A²=CR³, and A³=N;

R¹ is selected from the group consisting of H, C₁-C₈ alkyl, C₂-C₈ alkenyl, C₂-C₈ alkynyl, or C₃-C₁₀ cycloalkyl, wherein each alkyl, alkenyl, alkynyl, or cycloalkyl is independently optionally substituted with at least one selected from the group consisting of F, Cl, Br, I, C₁-C₈ alkyl, C₁-C₈ haloalkyl, and C₃-C₈ cycloalkyl;

R² is selected from the group consisting of H, C₁-C₈ alkyl, C₂-C₈ alkenyl, C₂-C₈ alkynyl, or C₃-C₁₀ cycloalkyl, wherein each alkyl, alkenyl, alkynyl, or cycloalkyl is independently optionally substituted with at least one selected from the group consisting of F, Cl, Br, I, C₁-C₈ alkyl, C₁-C₈ haloalkyl, and C₃-C₈ cycloalkyl;
or R¹ and R² combine with the N atom to which they are bound to form 3- to 8-membered heterocyclyl, wherein each heterocyclyl is independently optionally substituted with at least one substituent selected from the group consisting of F, Cl, Br, I, C₁-C₈ alkyl, C₁-C₈ haloalkyl, C₂-C₈ alkenyl, C₂-C₈ alkenyl, phenyl, heteroaryl, and (aryl)-C₁-C₆ alkyl, wherein two substituents bound to the same atom of the heterocyclyl can combine to form C₂-C₇ alkylene which is optionally substituted with at least one substituent selected from the group consisting of F, Cl, Br, I, C₁-C₈ alkyl, C₁-C₈ haloalkyl, C₂-C₈ alkenyl, C₂-C₈ alkenyl, phenyl, heteroaryl, and (aryl)-C₁-C₆ alkyl, and wherein two substituents bound to distinct atoms of the heterocyclyl can combine to form C₁-C₇ alkylene which is optionally substituted with at least one substituent selected from the group consisting of F, Cl, Br, I, C₁-C₈ alkyl, C₁-C₈ haloalkyl, C₂-C₈ alkenyl, C₂-C₈ alkenyl, phenyl, heteroaryl, and (aryl)-C₁-C₆ alkyl;

Y is —(CH₂)ₙ—, wherein n is 0, 1, 2, 3, 4, 5, or 6, and wherein each CH₂ group in Y is independently optionally substituted with at least one selected from the group consisting of C₁-C₆ alkyl, C₁-C₆ haloalkyl, F, Cl, Br, and I;
X is selected from the group consisting of —CH₂—, —O—, —NH—, —N(C₁-C₆ alkyl)-, and —S—, wherein if n is 0, then X is —CH₂—, and wherein each CH₂ group in X is independently optionally substituted with at least one selected from the group consisting of C₁-C₆ alkyl, C₁-C₆ haloalkyl, F, Cl, Br, and I;

R³ is selected from the group consisting of H, C₁-C₄ alkyl, C₂-C₄ alkenyl, C₂-C₄ alkynyl, and C₃-C₆ cycloalkyl, wherein each alkyl, alkenyl, alkynyl or cycloalkyl is independently optionally substituted with at least one selected from the group consisting of C₁-C₄ alkyl, C₁-C₄ haloalkyl, F, Cl, Br, and I;

each of R^{4a}—R^{4e} is independently selected from the group consisting of H, F, Cl, Br, I, C₁-C₈ alkyl, C₁-C₈ haloalkyl, C₁-C₈ haloalkoxy, C₂-C₈ alkenyl, C₂-C₈ alkynyl, C₃-C₁₀ cycloalkyl, phenyl, phenoxy, and benzyloxy, wherein each alkyl, alkenyl, alkynyl, cycloalkyl, phenyl, or benzyl is independently optionally substituted with at least one selected from the group consisting of C₁-C₆ alkyl, C₁-C₆ haloalkyl, F, Cl, Br, and I, or R^{4a} and R^{4d} combine with the atoms to which they are bound to form C₄-C₈ cycloalkyl, phenyl, or heterocyclyl, wherein each cycloalkyl, phenyl, heterocyclyl, or heteroaryl is independently substituted with at least one substituent selected from the group consisting of F, Cl, Br, I, C₁-C₈ alkyl, C₁-C₈ haloalkyl, C₂-C₈ alkenyl, and C₂-C₈ alkynyl; and wherein the heterocyclyl is selected from the group consisting of pyrrolyl, furanyl and thiophenyl.

Embodiment 2 provides the compound of Embodiment 1, which is selected from the group consisting of:

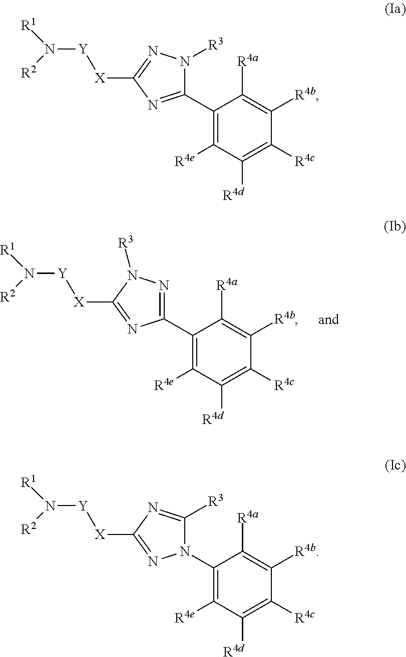

Embodiment 3 provides the compound of any of Embodiments 1-2, wherein —NR¹R² is an optionally substituted heterocyclyl selected from the group consisting of pyrrolidinyl, piperidinyl, morpholinyl, thiomorpholinyl, piperazinyl, N—(C₁-C₆ alkyl) piperidinyl, cyclohexyl-amino, and azepanyl.

Embodiment 4 provides the compound of any of Embodiments 1-3, wherein —NR¹R² is selected from the group consisting of:

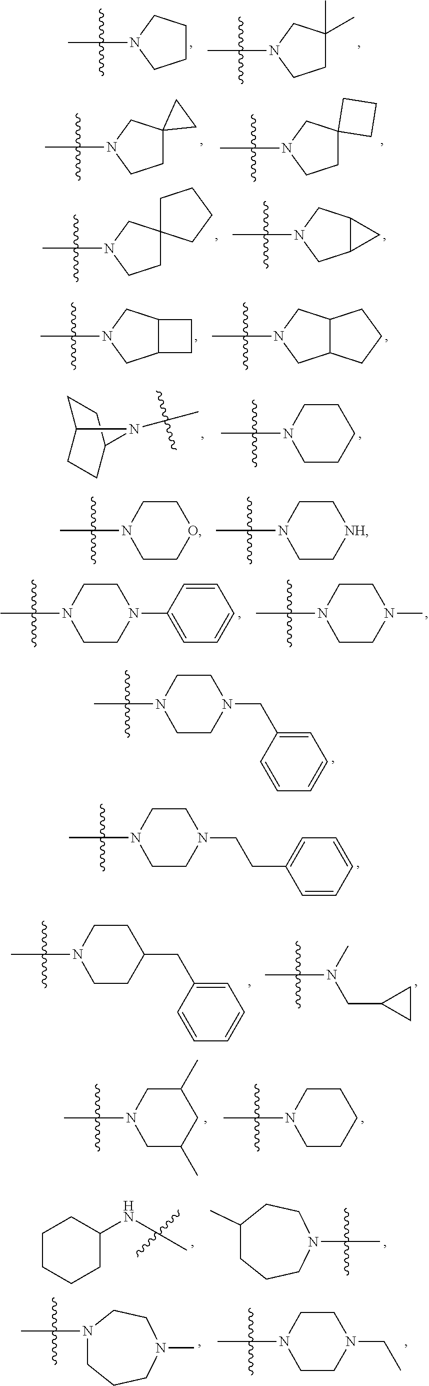

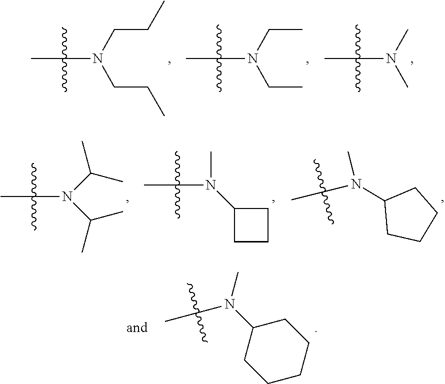

Embodiment 5 provides the compound of any of Embodiments 1-4, wherein

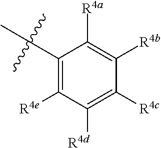

is selected from the group consisting of:

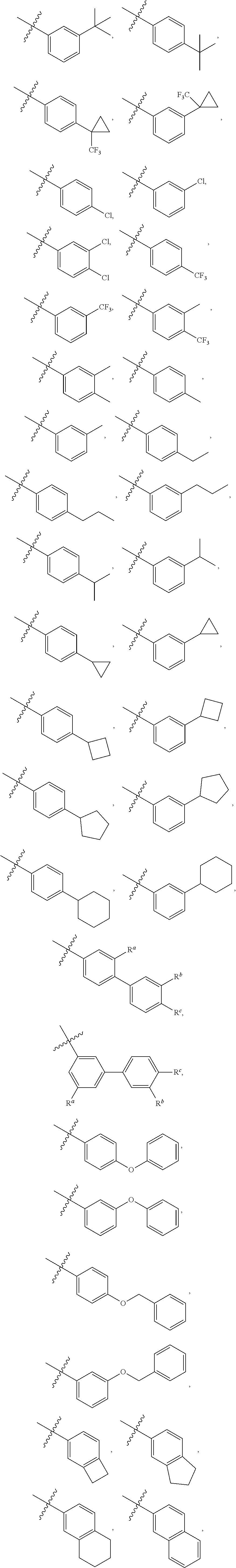

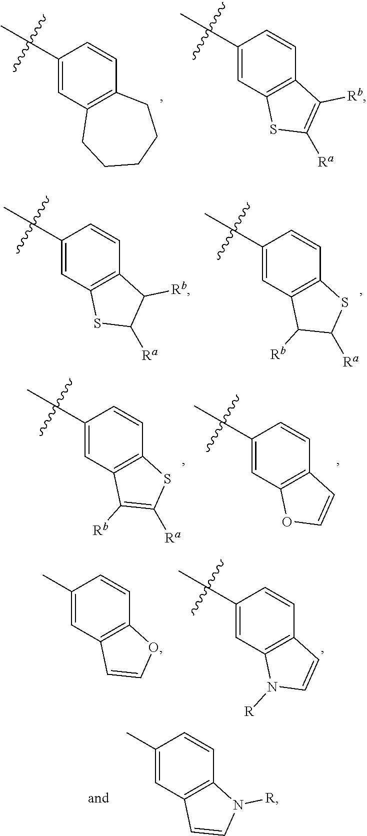

wherein each occurrence of R is independently H, C₁-C₆ alkyl, C₁-C₆ haloalkyl, and C₃-C₈ cycloalkyl, and wherein each occurrence of R^{a}, R^{b}, and R^{c} is independently H or C₁.

Embodiment 6 provides the compound of any of Embodiments 1-5, wherein R¹ and R² are each methyl.

Embodiment 7 provides the compound of any of Embodiments 1-6, wherein R³ is methyl, ethyl, isopropyl or cyclopropyl.

Embodiment 8 provides the compound of any of Embodiments 1-7, which is deuterated.

Embodiment 9 provides the compound of any of Embodiments 1-8, which is selected from the group consisting of: 1-((5-(4-(tert-butyl)phenyl)-1-methyl-1H-1,2,4-triazol-3-yl)methyl)piperidine; 4-((5-(4-(tert-butyl)phenyl)-1-methyl-1H-1,2,4-triazol-3-yl)methyl)morpholine; 1-((5-(4-(tert-butyl)phenyl)-1-methyl-1H-1,2,4-triazol-3-yl)methyl)-4-methylpiperazine; 1-((5-(4-(tert-butyl)phenyl)-1-methyl-1H-1,2,4-triazol-3-yl)methyl)-4-phenylpiperazine; 1-((5-(4-(tert-butyl)phenyl)-1-methyl-1H-1,2,4-triazol-3-yl)methyl)-4-phenethylpiperazine; 1-benzyl-4-((5-(4-(tert-butyl)phenyl)-1-methyl-1H-1,2,4-triazol-3-yl)methyl)piperazine; 4-benzyl-1-((5-(4-(tert-butyl)phenyl)-1-methyl-1H-1,2,4-triazol-3-yl)methyl)piperidine; 5-(4-(tert-butyl)phenyl)-1-methyl-3-(pyrrolidin-1-ylmethyl)-1H-1,2,4-triazole; 1-(5-(4-(tert-butyl)phenyl)-1-methyl-1H-1,2,4-triazol-3-yl)-N-(cyclopropylmethyl)-N-methylmethanamine; 1-methyl-3-(pyrrolidin-1-ylmethyl)-5-(4-(1-(trifluoromethyl)cyclopropyl)phenyl)-1H-1,2,4-triazole; 5-(3,4-dichlorophenyl)-1-methyl-3-(pyrrolidin-1-ylmethyl)-1H-1,2,4-triazole; 1-methyl-3-(pyrrolidin-1-ylmethyl)-5-(4-(trifluoromethyl)phenyl)-1H-1,2,4-triazole; 1-methyl-5-(3-methyl-4-(trifluoromethyl)phenyl)-3-(pyrrolidin-1-ylmethyl)-1H-1,2,4-triazole; 5-(4-isopropylphenyl)-1-methyl-3-(pyrrolidin-1-ylmethyl)-1H-1,2,4-triazole; 1-methyl-3-(pyrrolidin-1-ylmethyl)-5-(5,6,7,8-tetrahydronaphthalen-2-yl)-1H-1,2,4-triazole; 5-(3-(tert-butyl)phenyl)-1-methyl-3-(pyrrolidin-1-ylmethyl)-1H-1,2,4-triazole; 1-methyl-3-(pyrrolidin-1-ylmethyl)-5-(3-(1-(trifluoromethyl)cyclopropyl)phenyl)-1H-1,2,4-triazole; 5-(4-chlorophenyl)-1-methyl-3-(pyrrolidin-1-ylmethyl)-1H-1,2,4-triazole; 5-(3,4-dimethylphenyl)-1-methyl-3-(pyrrolidin-1-ylmethyl)-1H-1,2,4-triazole; 1-methyl-3-(pyrrolidin-1-ylmethyl)-5-(p-tolyl)-1H-1,2,4-triazole; 5-(4-cyclopropylphenyl)-1-methyl-3-(pyrrolidin-1-ylmethyl)-1H-1,2,4-triazole; 5-(2,3-dihydro-1H-inden-5-yl)-1-methyl-3-(pyrrolidin-1-ylmethyl)-1H-1,2,4-triazole; 5-(benzo[b]thiophen-6-yl)-1-methyl-3-(pyrrolidin-1-ylmethyl)-1H-1,2,4-triazole; 1-methyl-3-(pyrrolidin-1-ylmethyl)-5-(2,3′,4′-trichloro-[1,1′-biphenyl]-4-yl)-1H-1,2,4-triazole; 5-(2-chlorobenzo[b]thiophen-6-yl)-1-methyl-3-(pyrrolidin-1-ylmethyl)-1H-1,2,4-triazole; 5-(4-(tert-butyl)phenyl)-3-((3,3-dimethylpyrrolidin-1-yl)methyl)-1-methyl-1H-1,2,4-triazole; 2-((5-(4-(tert-butyl)phenyl)-1-methyl-1H-1,2,4-triazol-3-yl)methyl)-2-azaspiro[4.4]nonane; 5-((5-(4-(tert-butyl)phenyl)-1-methyl-1H-1,2,4-triazol-3-yl)methyl)-5-azaspiro[2.4]heptane; 6-((5-(4-(tert-butyl)phenyl)-1-methyl-1H-1,2,4-triazol-3-yl)methyl)-6-azaspiro[3.4]octane; 5-(4-(tert-butyl)phenyl)-1-ethyl-3-(pyrrolidin-1-ylmethyl)-1H-1,2,4-triazole; 5-(4-(tert-butyl)phenyl)-1-isopropyl-3-(pyrrolidin-1-ylmethyl)-1H-1,2,4-triazole; 5-(4-(tert-butyl)phenyl)-1-cyclopropyl-3-(pyrrolidin-1-ylmethyl)-1H-1,2,4-triazole; 3-(4-(tert-butyl)phenyl)-1-ethyl-5-(pyrrolidin-1-ylmethyl)-1H-1,2,4-triazole; 3-(4-(tert-butyl)phenyl)-1-isopropyl-5-(pyrrolidin-1-ylmethyl)-1H-1,2,4-triazole; 1-(4-(tert-butyl)phenyl)-5-methyl-3-(pyrrolidin-1-ylmethyl)-1H-1,2,4-triazole; 5-methyl-3-(pyrrolidin-1-ylmethyl)-1-(4-(1-(trifluoromethyl)cyclopropyl)phenyl)-1H-1,2,4-triazole; 1-(3,4-dichlorophenyl)-5-methyl-3-(pyrrolidin-1-ylmethyl)-1H-1,2,4-triazole; 5-methyl-1-(3-methyl-4-(trifluoromethyl)phenyl)-3-(pyrrolidin-1-ylmethyl)-1H-1,2,4-triazole; 5-([1,1′-biphenyl]-4-yl)-1-methyl-3-(pyrrolidin-1-ylmethyl)-1H-1,2,4-triazole; 5-([1,1′-biphenyl]-3-yl)-1-methyl-3-(pyrrolidin-1-ylmethyl)-1H-1,2,4-triazole; 1-methyl-5-(4-phenoxyphenyl)-3-(pyrrolidin-1-ylmethyl)-1H-1,2,4-triazole; 1-methyl-5-(3-phenoxyphenyl)-3-(pyrrolidin-1-ylmethyl)-1H-1,2,4-triazole; 5-(4-(benzyloxy)phenyl)-1-methyl-3-(pyrrolidin-1-ylmethyl)-1H-1,2,4-triazole; 5-(3-(benzyloxy)phenyl)-1-methyl-3-(pyrrolidin-1-ylmethyl)-1H-1,2,4-triazole; 5-(4-cyclohexylphenyl)-1-methyl-3-(pyrrolidin-1-ylmethyl)-1H-1,2,4-triazole; 3-((5-(4-(tert-butyl)phenyl)-1-methyl-1H-1,2,4-triazol-3-yl)methyl)-3-azabicyclo[3.1.0]hexane; 2-((5-(4-(tert-butyl)phenyl)-1-methyl-1H-1,2,4-triazol-3-yl)methyl)octahydrocyclopenta[c]pyrrole; (1s, 4s)-7-((5-(4-(tert-butyl)phenyl)-1-methyl-1H-1,2,4-triazol-3-yl)methyl)-7-azabicyclo[2.2.1]heptane; 1-(benzo[b]thiophen-6-yl)-5-methyl-3-(pyrrolidin-1-ylmethyl)-1H-1,2,4-triazole; 1-(benzo[b]thiophen-6-yl)-5-methyl-3-(pyrrolidin-1-ylmethyl)-1H-1,2,4-triazole; 5-methyl-3-(pyrrolidin-1-ylmethyl)-1-(5,6,7,8-tetrahydronaphthalen-2-yl)-1H-1,2,4-triazole; 1-(4-cyclohexylphenyl)-5-methyl-3-(pyrrolidin-1-ylmethyl)-1H-1,2,4-triazole; 1-([1,1′-biphenyl]-4-yl)-5-methyl-3-(pyrrolidin-1-ylmethyl)-1H-1,2,4-triazole; 3-(4-(tert-butyl)phenyl)-1-methyl-5-(pyrrolidin-1-ylmethyl)-1H-1,2,4-triazole; 3-(4-(tert-butyl)phenyl)-1-cyclopropyl-5-(pyrrolidin-1-ylmethyl)-1H-1,2,4-triazole; 1-((5-(4-(tert-butyl)phenyl)-1-methyl-1H-1,2,4-triazol-3-yl)methyl)piperazine; N-((5-(4-(tert-butyl)phenyl)-1-methyl-1H-1,2,4-triazol-3-yl)methyl)-N-methylcyclopropanamine; 5-(2,3-dihydrobenzo[b]thiophen-6-yl)-1-methyl-3-(pyrrolidin-1-ylmethyl)-1H-1,2,4-triazole; 5-(3-chlorophenyl)-1-methyl-3-(pyrrolidin-1-ylmethyl)-1H-1,2,4-triazole; 1-methyl-3-(pyrrolidin-1-ylmethyl)-5-(3-(trifluoromethyl)phenyl)-1H-1,2,4-triazole; 1-methyl-3-(pyrrolidin-1-ylmethyl)-5-(m-tolyl)-1H-1,2,4-triazole; 5-(4-propylphenyl)-1-methyl-3-(pyrrolidin-1-ylmethyl)-1H-1,2,4-triazole; 5-(4-(tert-butyl)phenyl)-3-((3,3-dimethylpyrrolidin-1-yl)methyl)-1-ethyl-1H-1,2,4-triazole; 5-((5-(4-(tert-butyl)phenyl)-1-ethyl-1H-1,2,4-triazol-3-yl)methyl)-5-azaspiro[2.4]heptane; 6-((5-(4-(tert-butyl)phenyl)-1-ethyl-1H-1,2,4-triazol-3-yl)methyl)-6-azaspiro[3.4]octane; 2-((5-(4-(tert-butyl)phenyl)-1-ethyl-1H-1,2,4-triazol-3-yl)methyl)-2-azaspiro[4.4]nonane; 2-((5-(4-(tert-butyl)phenyl)-1-ethyl-1H-1,2,4-triazol-3-yl)methyl)octahydrocyclopenta[c]pyrrole; 5-(4-(tert-butyl)phenyl)-3-((3,3-dimethylpyrrolidin-1-yl)methyl)-1-isopropyl-1H-1,2,4-triazole; 5-((5-(4-(tert-butyl)phenyl)-1-isopropyl-1H-1,2,4-triazol-3-yl)methyl)-5-azaspiro[2.4]heptane; 6-((5-(4-(tert-butyl)phenyl)-1-isopropyl-1H-1,2,4-triazol-3-yl)methyl)-6-azaspiro[3.4]octane; 2-((5-(4-(tert-butyl)phenyl)-1-isopropyl-1H-1,2,4-triazol-3-yl)methyl)-2-azaspiro[4.4]nonane; 2-((5-(4-(tert-butyl)phenyl)-1-isopropyl-1H-1,2,4-triazol-3-yl)methyl)octahydrocyclopenta[c]pyrrole; 5-(4-(tert-butyl)phenyl)-1-cyclopropyl-3-((3,3-dimethylpyrrolidin-1-yl)methyl)-1H-1,2,4-triazole; 5-((5-(4-(tert-butyl)phenyl)-1-cyclopropyl-1H-1,2,4-triazol-3-yl)methyl)-5-azaspiro[2.4]heptane; 6-((5-(4-(tert-butyl)phenyl)-1-cyclopropyl-1H-1,2,4-triazol-3-yl)methyl)-6-azaspiro[3.4]octane; 2-((5-(4-(tert-butyl)phenyl)-1-cyclopropyl-1H-1,2,4-triazol-3-yl)methyl)-2-azaspiro[4.4]nonane; and 2-((5-(4-(tert-butyl)phenyl)-1-cyclopropyl-1H-1,2,4-triazol-3-yl)methyl)octahydrocyclopenta[c]pyrrole; or a salt, solvate, isotopically labeled, enantiomer, diastereomer, and/or tautomer thereof.

Embodiment 10 provides a pharmaceutical composition comprising at least one pharmaceutically acceptable carrier and at least one compound of any of Embodiments 1-9.

Embodiment 11 provides the pharmaceutical composition of Embodiment 10, further comprising at least one therapeutic agent that activates or inhibits activity of a sigma receptor.

Embodiment 12 provides the pharmaceutical composition of Embodiment 11, wherein the at least one compound and the at least one additional therapeutic agent are co-formulated.

Embodiment 13 provides the pharmaceutical composition of any of Embodiments 11-12, wherein the at least one therapeutic agent treats or prevents pain.

Embodiment 14 provides a method of increasing or decreasing activity of at least one sigma receptor selected from the group consisting of sigma 1 receptor (S1R) and sigma 2 receptor (S2R), the method comprising contacting the at least one sigma receptor with at least one compound of any of Embodiments 1-9 and/or at least one pharmaceutical composition of any of Embodiments 10-13.

Embodiment 15 provides the method of Embodiment 14, wherein the at least one sigma receptor is in a cell.

Embodiment 16 provides the method of Embodiment 15, wherein the cell is mammalian.

Embodiment 17 provides the method of any of Embodiments 15-16, wherein the cell is in a subject.

Embodiment 18 provides the method of any of Embodiments 14-17, wherein the at least one compound and/or pharmaceutical composition binds to S1R selectively over S2R.

Embodiment 19 provides the method of any of Embodiments 14-17, wherein the at least one compound and/or pharmaceutical composition binds to S2R selectively over S1R.

Embodiment 20 provides the method of any of Embodiments 14-17, wherein the at least one compound and/or pharmaceutical composition binds to S1R and S2R with equal, or nearly equal, affinity.

Embodiment 21 provides the method of any of Embodiments 14-20, wherein the contacting promotes antinociception in the subject.

Embodiment 22 provides a method of inhibiting, treating, and/or preventing pain in a subject, the method comprising administering to the subject a therapeutically effective amount of at least one compound of any of Embodiments 1-9 and/or at least one pharmaceutical composition of any of Embodiments 10-13.

Embodiment 23 provides the method of Embodiment 22, wherein the at least one compound and/or pharmaceutical composition binds to S1R selectively over S2R.

Embodiment 24 provides the method of Embodiment 22, wherein the at least one compound and/or pharmaceutical composition binds to S2R selectively over S1R.

Embodiment 25 provides the method of Embodiment 22, wherein the at least one compound and/or pharmaceutical composition binds to S1R and S2R with equal, or nearly equal, affinity.

Embodiment 26 provides the method of any of Embodiments 22-25, wherein the administration promotes antinociception in the subject.

Embodiment 27 provides the method of any of Embodiments 22-26, wherein the pain comprises chronic pain, neuropathic pain, nociceptive pain, hyperalgesia, and/or allodynia.

Embodiment 28 provides the method of any of Embodiments 22-27, wherein the compound and/or pharmaceutical composition is the only therapeutically active agent administered to the subject.

Embodiment 29 provides the method of any of Embodiments 22-28, wherein the compound and/or pharmaceutical composition is the only therapeutically active agent administered to the subject in an amount sufficient to treat or prevent pain.

Embodiment 30 provides the method of any of Embodiments 22-27, wherein the subject is further administered at least one therapeutic agent that treats or prevents pain.

Embodiment 31 provides the method of Embodiment 30, wherein the at least one compound and the at least one additional therapeutic agent are co-administered to the subject.

Embodiment 32 provides the method of any of Embodiments 30-31, wherein the at least one compound and the at least one additional therapeutic agent are co-formulated.

Embodiment 33 provides the method of any of Embodiments 30-32, wherein the at least one therapeutic agent comprises an opioid agent.

Embodiment 34 provides the method of any of Embodiments 30-33, wherein administration of the opioid agent and at least one compound and/or composition to the subject allows for a lower amount of the opioid agent to be administered than if the subject is not administered the at least one compound and/or composition.

Embodiment 35 provides the method of any of Embodiments 30-34, wherein administration of the opioid agent and at least one compound and/or composition to the subject allows for use of an amount of the opioid agent that does not cause any significant side effect associated with opioid agent use.

Embodiment 36 provides a method of inhibiting, treating, and/or preventing a disease or disorder in a subject, the method comprising administering to the subject a therapeutically effective amount of at least one compound of any of Embodiments 1-9 and/or at least one pharmaceutical composition of any of Embodiments 10-13, wherein the disease or disorder is at least one selected from the group consisting of cancer and any other related proliferative diseases, neurological and neurodegenerative diseases, substance abuse disorders, depressive disorders, ocular disorders, and glaucoma.

Embodiment 37 provides the method of any of Embodiments 22-36, wherein the subject is a mammal.

Embodiment 38 provides the method of Embodiment 37, wherein the mammal is a human.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A compound of formula (I), or a salt, solvate, isotopically labeled, enantiomer, diastereoisomer, or tautomer thereof: 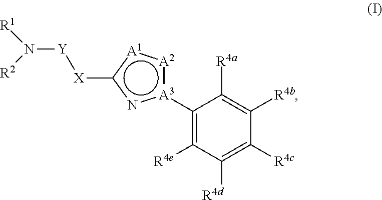
wherein:
A¹, A², and A³ are such that one applies: (a) A¹=N, A²=NR³, and A³=C; (b) A¹=NR³, A²=N, and A³=C; or (c) A¹=N, A²=CR³, and A³=N;
R¹ is selected from the group consisting of H, C₁-C₈ alkyl, C₂-C₈ alkenyl, C₂-C₈ alkynyl, or C₃-C₁₀ cycloalkyl, wherein each alkyl, alkenyl, alkynyl, or cycloalkyl is independently optionally substituted with at least one selected from the group consisting of F, Cl, Br, I, C₁-C₈ alkyl, C₁-C₈ haloalkyl, and C₃-C₈ cycloalkyl;
R² is selected from the group consisting of H, C₁-C₈ alkyl, C₂-C₈ alkenyl, C₂-C₈ alkynyl, or C₃-C₁₀ cycloalkyl, wherein each alkyl, alkenyl, alkynyl, or cycloalkyl is independently optionally substituted with at least one selected from the group consisting of F, Cl, Br, I, C₁-C₈ alkyl, C₁-C₈ haloalkyl, and C₃-C₈ cycloalkyl;
or R¹ and R² combine with the N atom to which they are bound to form 3- to 8-membered heterocyclyl,
wherein each heterocyclyl is independently optionally substituted with at least one substituent selected from the group consisting of F, Cl, Br, I, C₁-C₈ alkyl, C₁-C₈ haloalkyl, C₂-C₈ alkenyl, C₂-C₈ alkenyl, phenyl, heteroaryl, and (aryl)-C₁-C₆ alkyl,
wherein two substituents bound to the same atom of the heterocyclyl can combine to form C₂-C₇ alkylene which is optionally substituted with at least one substituent selected from the group consisting of F, Cl, Br, I, C₁-C₈ alkyl, C₁-C₈ haloalkyl, C₂-C₈ alkenyl, C₂-C₈ alkenyl, phenyl, heteroaryl, and (aryl)-C₁-C₆ alkyl, and
wherein two substituents bound to distinct atoms of the heterocyclyl can combine to form C₁-C₇ alkylene which is optionally substituted with at least one substituent selected from the group consisting of F, Cl, Br, I, C₁-C₈ alkyl, C₁-C₈ haloalkyl, C₂-C₈ alkenyl, C₂-C₈ alkenyl, phenyl, heteroaryl, and (aryl)-C₁-C₆ alkyl;
Y is —(CH₂)ₙ—, wherein n is 0, 1, 2, 3, 4, 5, or 6, and wherein each CH₂ group in Y is independently optionally substituted with at least one selected from the group consisting of C₁-C₆ alkyl, C₁-C₆ haloalkyl, F, Cl, Br, and I;
X is selected from the group consisting of —CH₂—, —O—, —NH—, —N(C₁-C₆ alkyl)-, and —S—,
wherein if n is 0, then X is —CH₂—, and wherein each CH₂ group in X is independently optionally substituted with at least one selected from the group consisting of C₁-C₆ alkyl, C₁-C₆ haloalkyl, F, Cl, Br, and I;
R³ is selected from the group consisting of H, C₁-C₄ alkyl, C₂-C₄ alkenyl, C₂-C₄ alkynyl, and C₃-C₆ cycloalkyl, wherein each alkyl, alkenyl, alkynyl or cycloalkyl is independently optionally substituted with at least one selected from the group consisting of C₁-C₄ alkyl, C₁-C₄ haloalkyl, F, Cl, Br, and I;
each of R^{4a}—R^{4e} is independently selected from the group consisting of H, F, Cl, Br, I, C₁-C₈ alkyl, C₁-C₈ haloalkyl, C₁-C₈ haloalkoxy, C₂-C₈ alkenyl, C₂-C₈ alkynyl, C₃-C₁₀ cycloalkyl, phenyl, phenoxy, and benzyloxy, wherein each alkyl, alkenyl, alkynyl, cycloalkyl, phenyl, or benzyl is independently optionally substituted with at least one selected from the group consisting of C₁-C₆ alkyl, C₁-C₆ haloalkyl, F, Cl, Br, and I,
or R^{4c} and R^{4d} combine with the atoms to which they are bound to form C₄-C₈ cycloalkyl, phenyl, or heterocyclyl, wherein each cycloalkyl, phenyl, heterocyclyl, or heteroaryl is independently substituted with at least one substituent selected from the group consisting of F, Cl, Br, I, C₁-C₈ alkyl, C₁-C₈ haloalkyl, C₂-C₈ alkenyl, and C₂-C₈ alkyny, and wherein the heterocyclyl is selected from the group consisting of pyrrolyl, furanyl and thiophenyl.

2. The compound of claim 1, which is selected from the group consisting of:

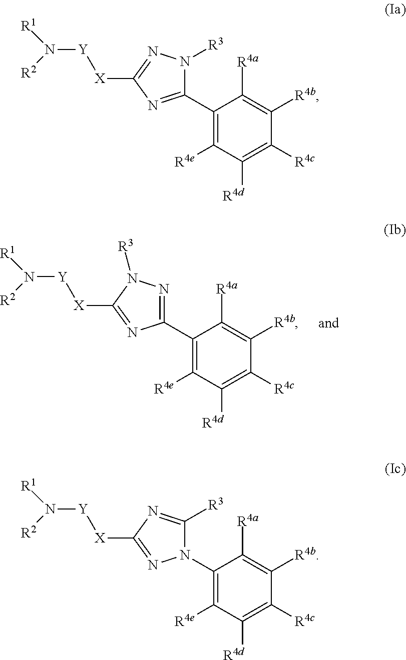

3. The compound of claim 1, wherein —NR¹R² is an optionally substituted heterocyclyl selected from the group consisting of pyrrolidinyl, piperidinyl, morpholinyl, thiomorpholinyl, piperazinyl, N—(C₁-C₆ alkyl) piperidinyl, cyclohexyl-amino, and azepanyl.

4. The compound of claim 1, wherein —NR¹R² is selected from the group consisting of:

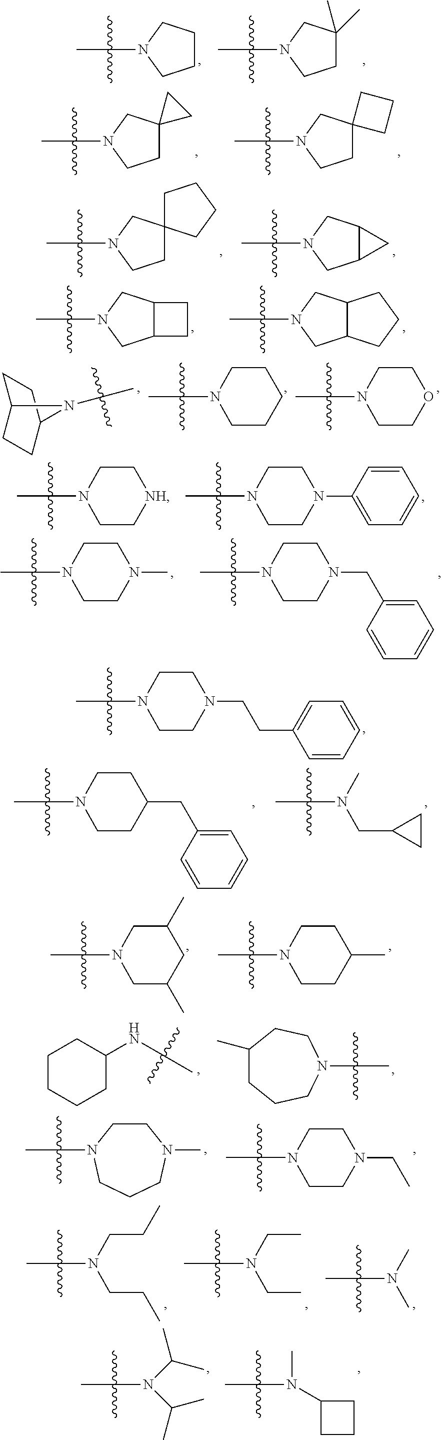

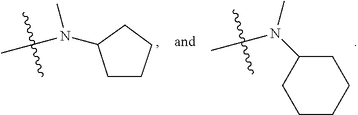

5. The compound of claim 1, wherein 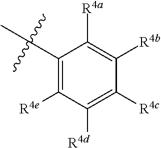
is selected from the group consisting of: 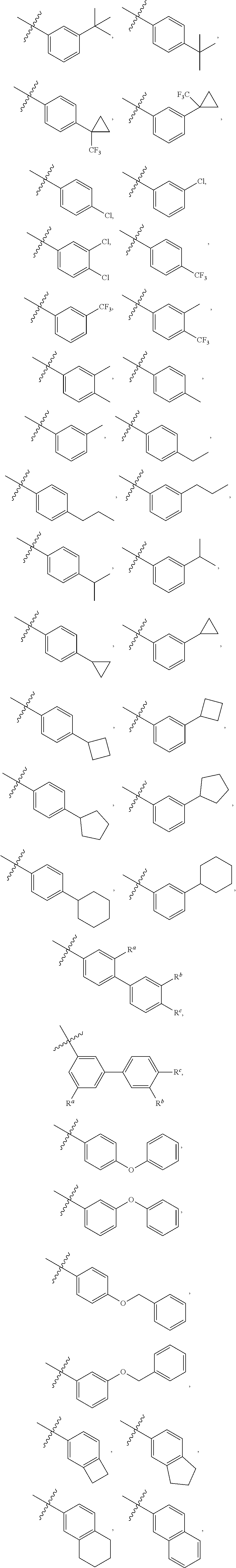 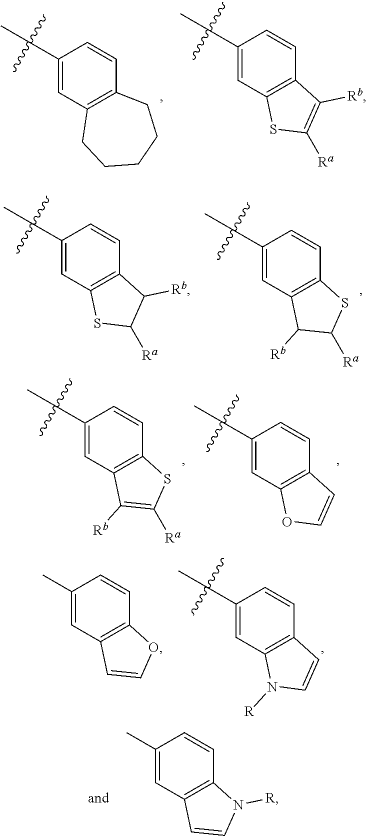
wherein each occurrence of R is independently H, C₁-C₆ alkyl, C₁-C₆ haloalkyl, and C₃-C₈ cycloalkyl, and wherein each occurrence of R^{a}, R^{b}, and R^{c} is independently H or Cl.

6. The compound of claim 1, wherein at least one of the following applies:
(a) R¹ and R² are each methyl; and
(b) R³ is methyl, ethyl, isopropyl or cyclopropyl.

7. The compound of claim 1, which is selected from the group consisting of:
1-((5-(4-(tert-butyl)phenyl)-1-methyl-1H-1,2,4-triazol-3-yl)methyl)piperidine;
4-((5-(4-(tert-butyl)phenyl)-1-methyl-1H-1,2,4-triazol-3-yl)methyl)morpholine;
1-((5-(4-(tert-butyl)phenyl)-1-methyl-1H-1,2,4-triazol-3-yl)methyl)-4-methylpiperazine;
1-((5-(4-(tert-butyl)phenyl)-1-methyl-1H-1,2,4-triazol-3-yl)methyl)-4-phenylpiperazine;
1-((5-(4-(tert-butyl)phenyl)-1-methyl-1H-1,2,4-triazol-3-yl)methyl)-4-phenethylpiperazine;
1-benzyl-4-((5-(4-(tert-butyl)phenyl)-1-methyl-1H-1,2,4-triazol-3-yl)methyl)piperazine;
4-benzyl-1-((5-(4-(tert-butyl)phenyl)-1-methyl-1H-1,2,4-triazol-3-yl)methyl)piperidine;
5-(4-(tert-butyl)phenyl)-1-methyl-3-(pyrrolidin-1-ylmethyl)-1H-1,2,4-triazole;
1-(5-(4-(tert-butyl)phenyl)-1-methyl-1H-1,2,4-triazol-3-yl)-N-(cyclopropylmethyl)-N-methylmethanamine;
1-methyl-3-(pyrrolidin-1-ylmethyl)-5-(4-(1-(trifluoromethyl)cyclopropyl)phenyl)-1H-1,2,4-triazole;
5-(3,4-dichlorophenyl)-1-methyl-3-(pyrrolidin-1-ylmethyl)-1H-1,2,4-triazole;
1-methyl-3-(pyrrolidin-1-ylmethyl)-5-(4-(trifluoromethyl)phenyl)-1H-1,2,4-triazole;
1-methyl-5-(3-methyl-4-(trifluoromethyl)phenyl)-3-(pyrrolidin-1-ylmethyl)-1H-1,2,4-triazole;
5-(4-isopropylphenyl)-1-methyl-3-(pyrrolidin-1-ylmethyl)-1H-1,2,4-triazole;
1-methyl-3-(pyrrolidin-1-ylmethyl)-5-(5,6,7,8-tetrahydronaphthalen-2-yl)-1H-1,2,4-triazole;
5-(3-(tert-butyl)phenyl)-1-methyl-3-(pyrrolidin-1-ylmethyl)-1H-1,2,4-triazole;
1-methyl-3-(pyrrolidin-1-ylmethyl)-5-(3-(1-(trifluoromethyl)cyclopropyl)phenyl)-1H-1,2,4-triazole;
5-(4-chlorophenyl)-1-methyl-3-(pyrrolidin-1-ylmethyl)-1H-1,2,4-triazole;
5-(3,4-dimethylphenyl)-1-methyl-3-(pyrrolidin-1-ylmethyl)-1H-1,2,4-triazole;
1-methyl-3-(pyrrolidin-1-ylmethyl)-5-(p-tolyl)-1H-1,2,4-triazole;
5-(4-cyclopropylphenyl)-1-methyl-3-(pyrrolidin-1-ylmethyl)-1H-1,2,4-triazole;
5-(2,3-dihydro-1H-inden-5-yl)-1-methyl-3-(pyrrolidin-1-ylmethyl)-1H-1,2,4-triazole;
5-(benzo[b]thiophen-6-yl)-1-methyl-3-(pyrrolidin-1-ylmethyl)-1H-1,2,4-triazole;
1-methyl-3-(pyrrolidin-1-ylmethyl)-5-(2,3′,4′-trichloro-[1,1′-biphenyl]-4-yl)-1H-1,2,4-triazole;
5-(2-chlorobenzo[b]thiophen-6-yl)-1-methyl-3-(pyrrolidin-1-ylmethyl)-1H-1,2,4-triazole;
5-(4-(tert-butyl)phenyl)-3-((3,3-dimethylpyrrolidin-1-yl)methyl)-1-methyl-1H-1,2,4-triazole;
2-((5-(4-(tert-butyl)phenyl)-1-methyl-1H-1,2,4-triazol-3-yl)methyl)-2-azaspiro[4.4]nonane;
5-((5-(4-(tert-butyl)phenyl)-1-methyl-1H-1,2,4-triazol-3-yl)methyl)-5-azaspiro[2.4]heptane;
6-((5-(4-(tert-butyl)phenyl)-1-methyl-1H-1,2,4-triazol-3-yl)methyl)-6-azaspiro[3.4]octane;
5-(4-(tert-butyl)phenyl)-1-ethyl-3-(pyrrolidin-1-ylmethyl)-1H-1,2,4-triazole;
5-(4-(tert-butyl)phenyl)-1-isopropyl-3-(pyrrolidin-1-ylmethyl)-1H-1,2,4-triazole;
5-(4-(tert-butyl)phenyl)-1-cyclopropyl-3-(pyrrolidin-1-ylmethyl)-1H-1,2,4-triazole;
3-(4-(tert-butyl)phenyl)-1-ethyl-5-(pyrrolidin-1-ylmethyl)-1H-1,2,4-triazole;
3-(4-(tert-butyl)phenyl)-1-isopropyl-5-(pyrrolidin-1-ylmethyl)-1H-1,2,4-triazole;
1-(4-(tert-butyl)phenyl)-5-methyl-3-(pyrrolidin-1-ylmethyl)-1H-1,2,4-triazole;
5-methyl-3-(pyrrolidin-1-ylmethyl)-1-(4-(1-(trifluoromethyl)cyclopropyl)phenyl)-1H-1,2,4-triazole;
1-(3,4-dichlorophenyl)-5-methyl-3-(pyrrolidin-1-ylmethyl)-1H-1,2,4-triazole;
5-methyl-1-(3-methyl-4-(trifluoromethyl)phenyl)-3-(pyrrolidin-1-ylmethyl)-1H-1,2,4-triazole;
5-([1,1′-biphenyl]-4-yl)-1-methyl-3-(pyrrolidin-1-ylmethyl)-1H-1,2,4-triazole;
5-([1,1′-biphenyl]-3-yl)-1-methyl-3-(pyrrolidin-1-ylmethyl)-1H-1,2,4-triazole;
1-methyl-5-(4-phenoxyphenyl)-3-(pyrrolidin-1-ylmethyl)-1H-1,2,4-triazole;
1-methyl-5-(3-phenoxyphenyl)-3-(pyrrolidin-1-ylmethyl)-1H-1,2,4-triazole;
5-(4-(benzyloxy)phenyl)-1-methyl-3-(pyrrolidin-1-ylmethyl)-1H-1,2,4-triazole;
5-(3-(benzyloxy)phenyl)-1-methyl-3-(pyrrolidin-1-ylmethyl)-1H-1,2,4-triazole;
5-(4-cyclohexylphenyl)-1-methyl-3-(pyrrolidin-1-ylmethyl)-1H-1,2,4-triazole;
3-((5-(4-(tert-butyl)phenyl)-1-methyl-1H-1,2,4-triazol-3-yl)methyl)-3-azabicyclo[3.1.0]hexane;
2-((5-(4-(tert-butyl)phenyl)-1-methyl-1H-1,2,4-triazol-3-yl)methyl)octahydrocyclopenta[c]pyrrole;
(1s, 4s)-7-((5-(4-(tert-butyl)phenyl)-1-methyl-1H-1,2,4-triazol-3-yl)methyl)-7-azabicyclo[2.2.1]heptane;
1-(benzo[b]thiophen-6-yl)-5-methyl-3-(pyrrolidin-1-ylmethyl)-1H-1,2,4-triazole;
5-methyl-3-(pyrrolidin-1-ylmethyl)-1-(5,6,7,8-tetrahydronaphthalen-2-yl)-1H-1,2,4-triazole;
1-(4-cyclohexylphenyl)-5-methyl-3-(pyrrolidin-1-ylmethyl)-1H-1,2,4-triazole;
1-([1,1′-biphenyl]-4-yl)-5-methyl-3-(pyrrolidin-1-ylmethyl)-1H-1,2,4-triazole;
3-(4-(tert-butyl)phenyl)-1-methyl-5-(pyrrolidin-1-ylmethyl)-1H-1,2,4-triazole;
3-(4-(tert-butyl)phenyl)-1-cyclopropyl-5-(pyrrolidin-1-ylmethyl)-1H-1,2,4-triazole;
1-((5-(4-(tert-butyl)phenyl)-1-methyl-1H-1,2,4-triazol-3-yl)methyl)piperazine;
N-((5-(4-(tert-butyl)phenyl)-1-methyl-1H-1,2,4-triazol-3-yl)methyl)-N-methylcyclopropanamine;
5-(2,3-dihydrobenzo[b]thiophen-6-yl)-1-methyl-3-(pyrrolidin-1-ylmethyl)-1H-1,2,4-triazole;
5-(3-chlorophenyl)-1-methyl-3-(pyrrolidin-1-ylmethyl)-1H-1,2,4-triazole;
1-methyl-3-(pyrrolidin-1-ylmethyl)-5-(3-(trifluoromethyl)phenyl)-1H-1,2,4-triazole;
1-methyl-3-(pyrrolidin-1-ylmethyl)-5-(m-tolyl)-1H-1,2,4-triazole;
5-(4-propylphenyl)-1-methyl-3-(pyrrolidin-1-ylmethyl)-1H-1,2,4-triazole;
5-(4-(tert-butyl)phenyl)-3-((3,3-dimethylpyrrolidin-1-yl)methyl)-1-ethyl-1H-1,2,4-triazole;
5-((5-(4-(tert-butyl)phenyl)-1-ethyl-1H-1,2,4-triazol-3-yl)methyl)-5-azaspiro[2.4]heptane;
6-((5-(4-(tert-butyl)phenyl)-1-ethyl-1H-1,2,4-triazol-3-yl)methyl)-6-azaspiro[3.4]octane;
2-((5-(4-(tert-butyl)phenyl)-1-ethyl-1H-1,2,4-triazol-3-yl)methyl)-2-azaspiro[4.4]nonane;
2-((5-(4-(tert-butyl)phenyl)-1-ethyl-1H-1,2,4-triazol-3-yl)methyl)octahydrocyclopenta[c]pyrrole;
5-(4-(tert-butyl)phenyl)-3-((3,3-dimethylpyrrolidin-1-yl)methyl)-1-isopropyl-1H-1,2,4-triazole;
5-((5-(4-(tert-butyl)phenyl)-1-isopropyl-1H-1,2,4-triazol-3-yl)methyl)-5-azaspiro[2.4]heptane;
6-((5-(4-(tert-butyl)phenyl)-1-isopropyl-1H-1,2,4-triazol-3-yl)methyl)-6-azaspiro[3.4]octane;
2-((5-(4-(tert-butyl)phenyl)-1-isopropyl-1H-1,2,4-triazol-3-yl)methyl)-2-azaspiro[4.4]nonane;
2-((5-(4-(tert-butyl)phenyl)-1-isopropyl-1H-1,2,4-triazol-3-yl)methyl)octahydrocyclopenta[c]pyrrole;
5-(4-(tert-butyl)phenyl)-1-cyclopropyl-3-((3,3-dimethylpyrrolidin-1-yl)methyl)-1H-1,2,4-triazole;
5-((5-(4-(tert-butyl)phenyl)-1-cyclopropyl-1H-1,2,4-triazol-3-yl)methyl)-5-azaspiro[2.4]heptane;
6-((5-(4-(tert-butyl)phenyl)-1-cyclopropyl-1H-1,2,4-triazol-3-yl)methyl)-6-azaspiro[3.4]octane;
2-((5-(4-(tert-butyl)phenyl)-1-cyclopropyl-1H-1,2,4-triazol-3-yl)methyl)-2-azaspiro[4.4]nonane; and
2-((5-(4-(tert-butyl)phenyl)-1-cyclopropyl-1H-1,2,4-triazol-3-yl)methyl)octahydrocyclopenta[c]pyrrole;
or a salt, solvate, isotopically labeled, enantiomer, diastereomer, and/or tautomer thereof.

8. A pharmaceutical composition comprising at least one pharmaceutically acceptable carrier and at least one compound of claim 1.

9. The pharmaceutical composition of claim 8, further comprising at least one therapeutic agent that activates or inhibits activity of a sigma receptor, optionally wherein the at least one therapeutic agent treats or ameliorates pain.

10. A method of increasing or decreasing activity of at least one sigma receptor selected from the group consisting of sigma 1 receptor (SlR) and sigma 2 receptor (S2R), the method comprising contacting the at least one sigma receptor with at least one compound of claim 1, wherein the contacting optionally promotes antinociception in the subject.

11. The method of claim 10, wherein the at least one sigma receptor is in a cell, optionally wherein at least one applies: the cell is mammalian, the cell is in a subject.

12. The method of claim 10, wherein one of the following applies:
(a) the at least one compound binds to S1R selectively over S2R;
(b) the at least one compound binds to S2R selectively over S1R; or
(c) the at least one compound binds to S1R and S2R with equal, or nearly equal, affinity.

13. A method of inhibiting, treating, or ameliorating pain in a subject, the method comprising administering to the subject a therapeutically effective amount of at least one compound of claim 1.

14. The method of claim 13, wherein one of the following applies:
(a) the at least one compound binds to S1R selectively over S2R;
(b) the at least one compound binds to S2R selectively over S1R; or
(c) the at least one compound binds to S1R and S2R with equal, or nearly equal, affinity.

15. The method of claim 13, wherein the administration promotes antinociception in the subject.

16. The method of claim 13, wherein the pain comprises chronic pain, neuropathic pain, nociceptive pain, hyperalgesia, or allodynia.

17. The method of claim 13, wherein the compound is the only therapeutically active agent administered to the subject, or the compound is the only therapeutically active agent administered to the subject in an amount sufficient to treat or prevent pain.

18. The method of claim 13, wherein the subject is further administered at least one therapeutic agent that treats or prevents pain, optionally wherein the at least one therapeutic agent is co-administered, and further optionally co-formulated, with the at least one compound.

19. The method of claim 18, wherein the at least one therapeutic agent comprises an opioid agent, optionally wherein administration of the opioid agent and the at least one compound to the subject allows for at least one of:
(a) a lower amount of the opioid agent to be administered than if the subject is not administered the at least one compound; and
(b) use of an amount of the opioid agent that does not cause any significant side effect associated with opioid agent use.

20. A method of inhibiting, treating, or ameliorating a disease or disorder in a subject, the method comprising administering to the subject a therapeutically effective amount of at least one compound of claim 1, wherein the disease or disorder is at least one selected from the group consisting of cancer and any other related proliferative diseases, neurological and neurodegenerative diseases, substance abuse disorders, depressive disorders, ocular disorders, and glaucoma, optionally wherein the subject is a mammal, further optionally wherein the mammal is a human.
